(19) United States
(12) Patent Application Publication
(10) Pub. No.: US 2022/0062352 A1
(43) Pub. Date: Mar. 3, 2022

(54) COMPOSITIONS FOR THE TREATMENT OF CONDITIONS

(71) Applicant: Demata Therapeutics, LLC, San Diego, CA (US)

(72) Inventors: Gerald Thomas Proehl, San Diego, CA (US); Christopher Joseph Nardo, San Diego, CA (US)

(21) Appl. No.: 17/298,918

(22) PCT Filed: Dec. 2, 2019
(86) PCT No.: PCT/US2019/064050

§ 371 Date: Jun. 1, 2021

Related U.S. Application Data
(60) Provisional application No. 62/774,738, filed on Dec. 3, 2018.

(51) Int. Cl. A61K 35/655; A61K 45/06; A61P 17/06

(52) U.S. Cl. CPC A61K 35/655; A61P 17/06; A61K 45/06

(57) ABSTRACT

The present disclosure relates to the treatment of diseases or conditions in a subject, including skin conditions or diseases, comprising applying to the skin of the subject a first composition comprising Spongilla, and a second composition comprising a therapeutically effective amount of one or more drugs, wherein said drugs are selected from a chemical compound, a mixture of chemical compounds, a biological macromolecule, or an extract made from biological materials. Further provided are such methods wherein the one or more biological macromolecules is selected from a recombinant protein, a fusion protein, an antibody, a monoclonal antibody, a humanized monoclonal antibody, a bivalent antibody, an antibody fragment, an antibody-drug conjugate, a Fc fragment, a Fab fragment, a Fab′ fragment, a (Fab′)2 fragment, a Fv fragment, and a scFv fragment.

### FIELD

The present disclosure relates to the treatment of a condition in a subject, including a skin condition, comprising applying to the skin of the subject a first composition comprising Spongilla, and a second composition comprising a therapeutically effective amount of one or more drugs. The disclosure also relates to a products or kits for the treatment of skin conditions in a subject comprising a first composition comprising Spongilla, and a second composition comprising a therapeutically effective amount of one or more drugs. The disclosure also relates to methods for delivering a drug into the skin of a subject, including the dermis, comprising applying to the skin of the subject a first composition comprising Spongilla, and a second composition comprising a therapeutically effective amount of one or more drugs. The disclosure also relates to methods for treating or preventing one or more skin conditions or diseases in a subject comprising delivering a therapeutically or prophylactically effective amount of a drug to an intradermal compartment of the subject's skin, such as the dermis, by applying to the skin of the subject a first composition comprising Spongilla, and a second composition comprising a therapeutically effective amount of one or more drugs. The disclosure also relates to methods for administering a drug or other active agent to a subject, comprising applying to skin of the subject a first composition containing an effective amount of the drug or active agent, a second composition comprising Spongilla. The disclosure also relates to methods for enhancing skin permeation of a topically applied pharmacologically active compound which otherwise has a low rate of skin penetration, comprising applying to the skin of the subject a first composition comprising Spongilla, followed by application of a second composition to the skin of the subject, wherein said second composition comprises a therapeutically effective amount of said pharmacologically active compound, such as a drug, such as a chemical compound, a mixture of chemical compounds, a biological macromolecule, or an extract made from biological materials. In another aspect is provided any of the methods disclosed herein, wherein said one or more drugs is a biological macromolecule. In another aspect is provided any of the methods disclosed herein, wherein the one or more drugs is a chemical compound. In another aspect is provided any of the methods disclosed herein, wherein the one or more drugs is a mixture of chemical compounds. In another aspect is provided any of the methods disclosed herein, wherein the one or more drugs is a biological macromolecule. In another aspect is provided any of the methods disclosed herein, wherein the one or more drugs is an extract made from biological materials. In some embodiments, the subject is a mammal, including but not limited to humans, dogs, cats, cattle, sheep, and goats. In some embodiments, the subject is a human.

### BACKGROUND

Skin conditions in subjects to be treated or prevented according to the methods disclosed herein, including in human subjects, include, but are not limited to, plaque psoriasis, atopic dermatitis, acne vulgaris, acne rosacea type 1, acne rosacea type 2, psoriasis, hyperhidrosis, alopecia areata, androgenic alopecia, keloids, hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, melasma, rheumatoid arthritis, lichen planus, pityriasis rubra pilaris, ichthyosis, and palmoplantar pustulosis, can be difficult to treat. In some cases, these diseases or conditions are treated by administering to the subject one or more drugs, such as a chemical compound, a mixture of chemical compounds, a biological macromolecule, or an extract made from biological materials. In some cases, the drug is a biological macromolecule, such as a recombinant protein, a fusion protein, an antibody, a monoclonal antibody, a humanized monoclonal antibody, a bivalent antibody, an antibody fragment, an antibody-drug conjugate, a Fc fragment, a Fab fragment, a Fab′ fragment, a (Fab′)2 fragment, a Fv fragment, and a scFv fragment. In most cases, drugs that used to treat such diseases are systemically administered to a subject in need thereof, such as by intravenous administration. However, systemic administration of such drugs for the treatment of a skin disease or condition in a subject often leads to undesired side effects or adverse events. Such undesired side effects or adverse events may be avoided by topically administering the drug to the area of the skin in the subject in need to treatment, but proper usage of topical therapies is often more complex than is the case for oral therapies and adherence may be a particularly significant issue for topical therapies. One of the complexities related to the use of topically applied drugs can be the difficulty in delivering a therapeutically effective amount of the drug to the desired site of action in the skin of the subject, such as the dermis. Some drugs, such as biological macromolecules, or an extract made from biological materials, do not sufficiently penetrate the skin of the subject so using them topically does not deliver a therapeutically effective amount of the drugs to the desired site of action in order to treat such skin diseases or conditions. Furthermore, poor subject adherence to treatment regimens using topical therapies, development of resistance to medications, and increased costs may contribute to treatment failure. Therefore, there is a need for improved methods of treating or preventing skin conditions or diseases in a subject using one or more drugs in which the one or more drugs is topically applied to the skin of the subject, avoids the systemic administration of the drugs that can lead to undesired side effects or adverse events, and permits a therapeutically effective amount of the one or more drugs to penetrate the skin of the subject so that a therapeutically effective amount of the one or more drugs is delivered to the desired site of action, such as the dermis of the subject. Such improved methods have the advantage of avoiding undesired side effects or adverse events associated with the systemic administration of drugs and allows the topical use of drugs with paradigms that will improve subject compliance and lead to improved treatment outcomes in such subjects.

A number of skin conditions in subjects have been treated with the topical application of materials derived from naturally occurring sponges, such as Spongilla lacustris. Some materials derived from Spongilla are promoted for the treatment of certain skin conditions, such as acne vulgaris, and as a cosmetic. The Spongilla contains organic and inorganic compounds. The total lipid content is approximately 5% of the biomass of the dried sponge and the protein is composed of spongin or sclerotized collagen. The polysaccharides and N-acetyl-D-glucosamine (NAG) are part of chitin and chitosan that has been reported to be an important component within the skeletal fibers of Spongilla lacustris and detected 750±1.5 μg N-acetyl-D-glucosamine per 1 mg of spicule-free skeleton. Chitin and chitosan are described as a family of linear polysaccharides consisting of varying amounts of α or β (1-4) linked residues of N-acetyl-2 amino-2-deoxy-D-glucose and 2-amino-2-deoxy-D glucose residues. In α-chitin, the chains are arranged in sheets or stacks, the chains in any one sheet having the same direction or ‘sense’. In β-chitin, adjacent sheets along the c axis have the same direction; the sheets are parallel, while in α-chitin adjacent sheets along the c axis have the opposite direction, they are antiparallel. Chitin is deacetylated into chitosan and can be further degraded into N-acetyl-D-glucosamine (NAG) units. Chitosan preparations are classified into native chitosan, chitosan formulations, complexes and derivatives with other substances. Chitosan can be used to prevent or treat wound and burn infections not only because of its intrinsic antimicrobial properties, but also by virtue of its ability to deliver extrinsic antimicrobial agents to wounds and burns. Chitosan is water- insoluble and highly viscous in dilute acidic solutions. Soluble chitosan oligosaccharides were found to be instrumental in suppressing the LPS-induced nuclear factor kappa-light-chain-enhancer of activated B cell (NF-κB)-dependent inflammatory gene expression, and this was associated with reduced nuclear translocation of NF-κB. Chitosan has also been demonstrated to have an antimicrobial effect against P. acnes and S. aureus. Chitosan of differing molecular weight (MW) were tested on antibacterial activity, chitosan of low MW (50-190 kDa), medium MW (190-310 kDa), and high MW (310-375+kDa). In a clinical study with acne vulgaris subjects, NAG quickly reduced the number of acne lesions over an 8-week period and was better tolerated by the subjects than 10% benzoyl peroxide. Concentrations of 2.5, 5, 10, and 20 μg/mL were tested against P. acnes, with high molecular weight having a greater effect against the gram-positive bacteria P. acnes.

### SUMMARY OF THE INVENTION

The inventors of the subject matter disclosed herein have discovered that an important component of materials derived from Spongilla are the siliceous spicules that comprise the skeletal structure of Spongilla. The inventors have discovered the spicules penetrate the stratum corneum of the skin of a subject during application and promote the penetration of topically-applied drugs, such as chemical compounds, a mixture of chemical compounds, a biological macromolecule, or an extract made from biological materials into the skin of the subject, such as the dermis, to permit or improve the delivery of a therapeutically effective amount of the drug to the site of action necessary to effectively treat skin diseases or conditions in the subject. The inventors of the subject matter disclosed herein have also discovered that the spicules derived from Spongilla are useful in facilitating and permitting certain therapeutic compounds and compositions to penetrate into the skin of subjects to which the spicules are applied, which compounds and compositions would otherwise not be able to penetrate the skin of the subject in order to reach their therapeutic targets and treat certain skin conditions. Among the compounds and compositions that may better penetrate the skin in the presence of materials derived from Spongilla are products comprising one or more chemical compounds, a mixture of chemical compounds, one or more biological macromolecules, or an extract made from biological materials. The inventors of the subject matter disclosed herein have discovered the materials comprising Spongilla used herein may comprise all organic and/or inorganic compounds and materials that are part of the naturally-occurring Spongilla, or may include only a portion of the organic and/or inorganic compounds and materials that are part of the naturally-occurring Spongilla.

In one aspect is provided a method of treating a disease or condition in a subject, comprising applying to the skin of the subject a first composition comprising Spongilla, and a second composition comprising a therapeutically effective amount of one or more drugs.

In one aspect is provided a method of treating a skin disease or condition in a subject, comprising applying to the skin of the subject a first composition comprising Spongilla, and a second composition comprising a therapeutically effective amount of one or more drugs.

In one aspect is provided a method for delivering a drug into the skin of a subject, comprising applying to the skin of the subject a first composition comprising Spongilla, and a second composition comprising a therapeutically effective amount of one or more drugs.

In one aspect is provided a method for treating or preventing one or more skin conditions or diseases in a subject comprising delivering a therapeutically or prophylactically effective amount of a drug to an intradermal compartment of the subject's skin by applying to the skin of the subject a first composition comprising Spongilla, and a second composition comprising a therapeutically effective amount of one or more drugs.

In one aspect is provided a method for administering a drug or other active agent to a subject, comprising applying to skin of the subject a first composition containing an effective amount of the drug or active agent, a second composition comprising Spongilla.

In one aspect is provided a method for enhancing skin permeation of a topically applied pharmacologically active compound which otherwise has a low rate of skin penetration, comprising applying to the skin of the subject a first composition comprising Spongilla, followed by application of a second composition to the skin of the subject, wherein said second composition comprises a therapeutically effective amount of said pharmacologically active compound, such as a chemical compound, a mixture of chemical compounds, a biological macromolecule, or an extract made from biological materials. In another aspect is provided any of the methods disclosed herein, wherein said one or more drugs is a biological macromolecule. In another aspect is provided any of the methods disclosed herein, wherein the one or more drugs is a chemical compound. In another aspect is provided any of the methods disclosed herein, wherein the one or more drugs is a mixture of chemical compounds. In another aspect is provided any of the methods disclosed herein, wherein the one or more drugs is a biological macromolecule. In another aspect is provided any of the methods disclosed herein, wherein the one or more drugs is an extract made from biological materials.

In one aspect is provided methods to treat, reduce the symptoms of, ameliorate the symptoms of, delay the onset of, or otherwise pharmaceutically address plaque psoriasis, psoriatic arthritis, atopic dermatitis, acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, and melasma.

### DETAILED DESCRIPTION

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

As used herein, the term “about” means either within plus or minus 10% of the provided value, or rounded to the nearest significant figure, in all cases inclusive of the provided value. Where ranges are provided, they are inclusive of the boundary values.

As used herein, the terms “applied,” “applying,” “administration,” “administering,” and “used” means the delivery of a composition disclosed herein to a subject, in particular to the skin of the subject, by an administration route including, but not limited to, intraperitoneal, subcutaneous, intramuscular, topically, or any combinations thereof. In some embodiments disclosed herein, the compositions disclosed herein are administered to the subject, in particular to the skin of the subject, by topical administration.

As used herein, the term “aspect ratio” means with respect to the particles of Spongilla described herein the ratio between the average length of the particles to the average diameter of the particles.

As used herein, the terms “combination” and “in combination with” mean the application, use, or administration of one or more of the compositions disclosed herein, sequentially or simultaneously. It includes dosing simultaneously, or within minutes or hours of each other, or on the same day, or on alternating days, or using the compositions disclosed herein on a daily basis, or multiple days per week, or weekly basis, for example, while administering another composition on the same day or alternating days or weeks or on a periodic basis during a time simultaneous therewith or concurrent therewith, or at least a part of the time during which the composition disclosed herein is applied, used or administered. For example, one or more of the compositions disclosed herein, could be applied, used, or administered to a subject every day or several days a week while the additional composition is applied, used or dosed on alternating days or alternating weeks or other periods of time, such as every 1, 2, 3, 4, 5, 6, 7, 8, 9, 10, 11. 12, 13, 14 or more days.

The term “Spongilla” as used herein means a genus of freshwater sponges in the family Spongillidae, including, but not limited to, Spongilla lacustris, S. fragilis Leidy, and Ephydatia fluviatilis. The term “Spongilla lacustris” as used herein means a species of sponge of the freshwater sponge family Spongillidae.

The terms “composition comprising Spongilla,” “powders comprising Spongilla, “materials comprising Spongilla, “Spongilla in the form of a powder,” and the like, as used herein, mean materials comprising Spongilla derived from raw Spongilla that is harvested and processed and may include all the various components of the Spongilla following harvest, including all organic and/or inorganic compounds and materials that are part of the naturally-occurring Spongilla, or may include only a portion of the organic and/or inorganic compounds and materials that are part of the naturally-occurring Spongilla. In one aspect is provided any of the methods or kits disclosed herein, wherein the Spongilla materials comprise all or substantially all the organic and inorganic materials derived from the naturally occurring Spongilla. In another aspect is provided any of the methods or kits disclosed herein, wherein the Spongilla materials comprise (a) only the spicules and any materials that are naturally associated with the spicules, or (b) substantially purified spicules and any materials that are naturally associated with the spicules, or (c) purified spicules and any materials that are naturally associated with the spicules that are a component part of naturally-occurring Spongilla. In another aspect is provided any of the methods or kits disclosed herein, wherein the Spongilla materials comprise only the spicules and any materials that are naturally associated with the spicules. In another aspect is provided any of the methods or kits disclosed herein, wherein the Spongilla materials comprise substantially purified spicules and any materials that are naturally associated with the spicules. In another aspect is provided any of the methods or kits disclosed herein, wherein the Spongilla materials comprise purified spicules and any materials that are naturally associated with the spicules that are a component part of naturally occurring Spongilla.

As used herein, the term “therapeutically effective amount” means that amount of the composition or combination of compositions being applied, used or administered to a subject that will treat, relieve, or prevent to some extent one or more of the symptoms of the disorder being treated.

In another aspect is provided any of the methods disclosed herein, wherein the disease or condition, including a skin disease or condition, is selected from plaque psoriasis, psoriatic arthritis, atopic dermatitis, acne vulgaris, acne rosacea type 1, acne rosacea type 2, psoriasis, hyperhidrosis, alopecia areata, androgenic alopecia, keloids, hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, melasma, rheumatoid arthritis, lichen planus, pityriasis rubra pilaris, ichthyosis, and palmoplantar pustulosis. In another aspect is provided any of the methods disclosed herein, wherein the disease or condition is plaque psoriasis. In another aspect is provided any of the methods disclosed herein, wherein the disease or condition is psoriatic arthritis. In another aspect is provided any of the methods disclosed herein, wherein the disease or condition is atopic dermatitis. In another aspect is provided any of the methods disclosed herein, wherein the disease or condition is acne vulgaris. In another aspect is provided any of the methods disclosed herein, wherein the disease or condition is acne rosacea type 1. In another aspect is provided any of the methods disclosed herein, wherein the disease or condition is acne rosacea type 2. In another aspect is provided any of the methods disclosed herein, wherein the disease or condition is psoriasis. In another aspect is provided any of the methods disclosed herein, wherein the disease or condition is hyperhidrosis. In another aspect is provided any of the methods disclosed herein, wherein the disease or condition is alopecia. In another aspect is provided any of the methods disclosed herein, wherein the disease or condition is areata. In another aspect is provided any of the methods disclosed herein, wherein the disease or condition is androgenic alopecia. In another aspect is provided any of the methods disclosed herein, wherein the disease or condition is the treatment of keloids. In another aspect is provided any of the methods disclosed herein, wherein the disease or condition is the treatment of hypertrophic scars. In another aspect is provided any of the methods disclosed herein, wherein the disease or condition is hidradenitis suppurativa. In another aspect is provided any of the methods disclosed herein, wherein the disease or condition is Raynaud phenomenon. In another aspect is provided any of the methods disclosed herein, wherein the disease or condition is post-herpetic neuralgia. In another aspect is provided any of the methods disclosed herein, wherein the disease or condition is Hailey-Hailey disease. In another aspect is provided any of the methods disclosed herein, wherein the disease or condition is IgA bullous dermatosis. In another aspect is provided any of the methods disclosed herein, wherein the disease or condition is epidermolysis bullosa Simplex Weber-Cockane. In another aspect is provided any of the methods disclosed herein, wherein the disease or condition is Darier disease. In another aspect is provided any of the methods disclosed herein, wherein the disease or condition is pachyonchia congenita. In another aspect is provided any of the methods disclosed herein, wherein the disease or condition is aquagenic keratoderma. In another aspect is provided any of the methods disclosed herein, wherein the disease or condition is notalgia paresthetic. In another aspect is provided any of the methods disclosed herein, wherein the disease or condition is pompholyx (dyshidrotic eczema). In another aspect is provided any of the methods disclosed herein, wherein the disease or condition is chromhidrosis and bromhidrosis. In another aspect is provided any of the methods disclosed herein, wherein the disease or condition is eccrine nevus. In another aspect is provided any of the methods disclosed herein, wherein the disease or condition is facial rhytides. In another aspect is provided any of the methods disclosed herein, wherein the disease or condition is atrohpic acne scars. In another aspect is provided any of the methods disclosed herein, wherein the disease or condition is melasma. In another aspect is provided any of the methods disclosed herein, wherein the disease or condition is rheumatoid arthritis. In another aspect is provided any of the methods disclosed herein, wherein the disease or condition is lichen planus. In another aspect is provided any of the methods disclosed herein, wherein the disease or condition is pityriasis rubra pilaris. In another aspect is provided any of the methods disclosed herein, wherein the disease or condition is ichthyosis. In another aspect is provided any of the methods disclosed herein, wherein the disease or condition is palmoplantar pustulosis.

In another aspect is provided any of the methods disclosed herein, wherein the one or more drugs is not a botulinum toxin, an antibiotic, an anti-inflammatory, an antiseptic, or an anesthetic. In another aspect is provided any of the methods disclosed herein, wherein the anti-inflammatory is not selected from non-steroidal anti-inflammatories and steroidal anti-inflammatories. In another aspect is provided any of the methods disclosed herein, wherein the one or more drugs is not a botulinum toxin. In another aspect is provided any of the methods disclosed herein, wherein the one or more drugs is not an antibiotic. In another aspect is provided any of the methods disclosed herein, wherein the one or more drugs is not an anti-inflammatory. In another aspect is provided any of the methods disclosed herein, wherein the one or more drugs is not an antiseptic. In another aspect is provided any of the methods disclosed herein, wherein the one or more drugs is not an anesthetic.

In another aspect is provided any of the methods disclosed herein, wherein said drug is selected from a T-cell co-stimulation modulator, an antagonist of one or more interleukin receptors, an antagonist of one or more interleukins, an interferon-gamma antagonist, a tissue necrosis factor-alpha antagonist, and a transforming growth factor-beta agonist. In another aspect is provided any of the methods disclosed herein, wherein said drug is a T-cell co-stimulation modulator. In another aspect is provided any of the methods disclosed herein, wherein said drug is an antagonist of one or more interleukin receptors. In another aspect is provided any of the methods disclosed herein, wherein said drug is an antagonist of one or more interleukins. In another aspect is provided any of the methods disclosed herein, wherein said drug is an interferon-gamma antagonist. In another aspect is provided any of the methods disclosed herein, wherein said drug is a tissue necrosis factor-alpha antagonist. In another aspect is provided any of the methods disclosed herein, wherein said drug is a transforming growth factor-beta agonist.

In another aspect is provided any of the methods disclosed herein, wherein said drug is an antagonist of one or more interleukin receptors. In another aspect is provided any of the methods disclosed herein, wherein said interleukin receptor is interleukin 1 receptor (IL-1R). In another aspect is provided any of the methods disclosed herein, wherein said interleukin receptor is IL-1RA. In another aspect is provided any of the methods disclosed herein, wherein said interleukin receptor is IL-1RB. In another aspect is provided any of the methods disclosed herein, wherein said interleukin receptor is IL-2R. In another aspect is provided any of the methods disclosed herein, wherein said interleukin receptor is IL-4R. In another aspect is provided any of the methods disclosed herein, wherein said interleukin receptor is IL-5R. In another aspect is provided any of the methods disclosed herein, wherein said interleukin receptor is IL-6R. In another aspect is provided any of the methods disclosed herein, wherein said interleukin receptor is IL-10R. In another aspect is provided any of the methods disclosed herein, wherein said interleukin receptor is IL-12R. In another aspect is provided any of the methods disclosed herein, wherein said interleukin receptor is IL-13R. In another aspect is provided any of the methods disclosed herein, wherein said interleukin receptor is IL-17R. In another aspect is provided any of the methods disclosed herein, wherein said interleukin receptor is IL-17RA. In another aspect is provided any of the methods disclosed herein, wherein said interleukin receptor is IL-21R. In another aspect is provided any of the methods disclosed herein, wherein said interleukin receptor is IL-22R. In another aspect is provided any of the methods disclosed herein, wherein said interleukin receptor is IL-23R. In another aspect is provided any of the methods disclosed herein, wherein said interleukin receptor is IL-31R. In another aspect is provided any of the methods disclosed herein, wherein said interleukin receptor is IL-35R.

In another aspect is provided any of the methods disclosed herein, wherein said drug is an antagonist of one or more interleukins. In another aspect is provided any of the methods disclosed herein, wherein said interleukin is IL-1. In another aspect is provided any of the methods disclosed herein, wherein said interleukin is IL-1A. In another aspect is provided any of the methods disclosed herein, wherein said interleukin is IL-1B. In another aspect is provided any of the methods disclosed herein, wherein said interleukin is IL-2. In another aspect is provided any of the methods disclosed herein, wherein said interleukin is IL-4. In another aspect is provided any of the methods disclosed herein, wherein said interleukin is IL-5. In another aspect is provided any of the methods disclosed herein, wherein said interleukin is IL-6. In another aspect is provided any of the methods disclosed herein, wherein said interleukin is IL-10. In another aspect is provided any of the methods disclosed herein, wherein said interleukin is IL-12. In another aspect is provided any of the methods disclosed herein, wherein said interleukin is IL-13. In another aspect is provided any of the methods disclosed herein, wherein said interleukin is IL-17. In another aspect is provided any of the methods disclosed herein, wherein said interleukin is IL-17A. In another aspect is provided any of the methods disclosed herein, wherein said interleukin is IL-21. In another aspect is provided any of the methods disclosed herein, wherein said interleukin is IL-22. In another aspect is provided any of the methods disclosed herein, wherein said interleukin is IL-23. In another aspect is provided any of the methods disclosed herein, wherein said interleukin is IL-31. In another aspect is provided any of the methods disclosed herein, wherein said interleukin is IL-35.

In another aspect is provided any of the methods disclosed herein, wherein the one or more drugs is selected from a chemical compound, a mixture of chemical compounds, a biological macromolecule, or an extract made from biological materials. In another aspect is provided any of the methods disclosed herein, wherein said one or more drugs is a biological macromolecule. In another aspect is provided any of the methods disclosed herein, wherein the one or more drugs is a chemical compound. In another aspect is provided any of the methods disclosed herein, wherein the one or more drugs is a mixture of chemical compounds. In another aspect is provided any of the methods disclosed herein, wherein the one or more drugs is a biological macromolecule. In another aspect is provided any of the methods disclosed herein, wherein the one or more drugs is an extract made from biological materials.

In another aspect is provided any of the methods disclosed herein, wherein said one or more biological macromolecules is selected from a recombinant protein, a fusion protein, an antibody, a monoclonal antibody, a humanized monoclonal antibody, a bivalent antibody, an antibody fragment, an antibody-drug conjugate, a Fc fragment, a Fab fragment, a Fab′ fragment, a (Fab′)2 fragment, a Fv fragment, and a scFv fragment. In another aspect is provided any of the methods disclosed herein, wherein the one or more biological macromolecules is a recombinant protein. In another aspect is provided any of the methods disclosed herein, wherein the one or more biological macromolecules is a fusion protein. In another aspect is provided any of the methods disclosed herein, wherein the one or more biological macromolecules is an antibody. In another aspect is provided any of the methods disclosed herein, wherein the one or more biological macromolecules is a monoclonal antibody. In another aspect is provided any of the methods disclosed herein, wherein the one or more biological macromolecules is a humanized monoclonal antibody. In another aspect is provided any of the methods disclosed herein, wherein the one or more biological macromolecules is a bivalent antibody. In another aspect is provided any of the methods disclosed herein, wherein the one or more biological macromolecules is an antibody fragment. In another aspect is provided any of the methods disclosed herein, wherein the one or more biological macromolecules is an antibody-drug conjugate. In another aspect is provided any of the methods disclosed herein, wherein the one or more biological macromolecules is a Fc fragment. In another aspect is provided any of the methods disclosed herein, wherein the one or more biological macromolecules is a Fab fragment. In another aspect is provided any of the methods disclosed herein, wherein the one or more biological macromolecules is a Fab′ fragment. In another aspect is provided any of the methods disclosed herein, wherein the one or more biological macromolecules is a (Fab′)2 fragment. In another aspect is provided any of the methods disclosed herein, wherein the one or more biological macromolecules is a Fv fragment. In another aspect is provided any of the methods disclosed herein, wherein the one or more biological macromolecules is a scFv fragment.

In another aspect is provided any of the methods disclosed herein, wherein said one or more biological molecules is an antibody. In another aspect is provided any of the methods disclosed herein, wherein said antibody is a monoclonal antibody. In another aspect is provided any of the methods disclosed herein, wherein said monoclonal antibody is a humanized antibody. In another aspect is provided any of the methods disclosed herein, wherein the antibody is selected from one or more subclasses consisting of IgG1, IgG2, and IgG4. In another aspect is provided any of the methods disclosed herein, wherein said antibody is selected from one or more subclasses consisting of IgG1, IgG1/kappa, IgG1/lambda, IgG2, IgG2/kappa, and IgG4. In another aspect is provided any of the methods disclosed herein, wherein the antibody is an IgG1 antibody. In another aspect is provided any of the methods disclosed herein, wherein the antibody is an IgG2 antibody. In another aspect is provided any of the methods disclosed herein, wherein the antibody is an IgG4 antibody. In another aspect is provided any of the methods disclosed herein, wherein said antibody is an IgG1/kappa antibody. In another aspect is provided any of the methods disclosed herein, wherein said antibody is an IgG1/lambda antibody. In another aspect is provided any of the methods disclosed herein, wherein said antibody is an IgG2 antibody. In another aspect is provided any of the methods disclosed herein, wherein said antibody is an IgG2/kappa antibody. In another aspect is provided any of the methods disclosed herein, wherein said antibody an IgG4antibody.

In another aspect is provided any of the methods disclosed herein, wherein said antibody is selected from secukinumab, ixekizumab, adalimumab, brodalumab, guselkumab, ustekinumab, sarilumab, dupilumab, tildrakizumab, lebrikizumab, mepolizumab, fezakinumab, nemolizumab, risankizumab, BMS 981164, CMJ112, tralokinumab, cemiplimab, infliximab, basiliximab, daclizumab, efalizumab, ustekinumab, certolizumab pegol, ABX-IL8, omalizumab, mirikizumab, and MSB0010841 (ALX-0761). In another aspect is provided any of the methods disclosed herein, wherein the antibody is secukinumab. In another aspect is provided any of the methods disclosed herein, wherein the antibody is ixekizumab. In another aspect is provided any of the methods disclosed herein, wherein the antibody is adalimumab. In another aspect is provided any of the methods disclosed herein, wherein the antibody is brodalumab. In another aspect is provided any of the methods disclosed herein, wherein the antibody is guselkumab. In another aspect is provided any of the methods disclosed herein, wherein the antibody is ustekinumab. In another aspect is provided any of the methods disclosed herein, wherein the antibody is sarilumab. In another aspect is provided any of the methods disclosed herein, wherein the antibody is dupilumab. In another aspect is provided any of the methods disclosed herein, wherein the antibody is tildrakizumab. In another aspect is provided any of the methods disclosed herein, wherein the antibody is lebrikizumab. In another aspect is provided any of the methods disclosed herein, wherein the antibody is mepolizumab. In another aspect is provided any of the methods disclosed herein, wherein the antibody is fezakinumab. In another aspect is provided any of the methods disclosed herein, wherein the antibody is nemolizumab. In another aspect is provided any of the methods disclosed herein, wherein the antibody is risankizumab. In another aspect is provided any of the methods disclosed herein, wherein the antibody is BMS 981164. In another aspect is provided any of the methods disclosed herein, wherein the antibody is CMJ112. In another aspect is provided any of the methods disclosed herein, wherein the antibody is tralokinumab. In another aspect is provided any of the methods disclosed herein, wherein the antibody is cemiplimab. In another aspect is provided any of the methods disclosed herein, wherein the antibody is infliximab. In another aspect is provided any of the methods disclosed herein, wherein the antibody is basiliximab. In another aspect is provided any of the methods disclosed herein, wherein the antibody is daclizumab. In another aspect is provided any of the methods disclosed herein, wherein the antibody is efalizumab. In another aspect is provided any of the methods disclosed herein, wherein the antibody is ustekinumab. In another aspect is provided any of the methods disclosed herein, wherein the antibody is certolizumab pegol. In another aspect is provided any of the methods disclosed herein, wherein the antibody is ABX-IL8. In another aspect is provided any of the methods disclosed herein, wherein the antibody is omalizumab. In another aspect is provided any of the methods disclosed herein, wherein the antibody is mirikizumab. In another aspect is provided any of the methods disclosed herein, wherein the antibody is MSB0010841 (ALX-0761).

In another aspect is provided any of the methods disclosed herein, wherein said biologic macromolecules is a protein. In another aspect is provided any of the methods disclosed herein, wherein said protein is selected from etanercept, abatacept, rilonacept, and anakinra. In another aspect is provided any of the methods disclosed herein, wherein the protein is etanercept. In another aspect is provided any of the methods disclosed herein, wherein the protein is abatacept. In another aspect is provided any of the methods disclosed herein, wherein the protein is rilonacept. In another aspect is provided any of the methods disclosed herein, wherein the protein is anakinra.

In another aspect is provided any of the methods disclosed herein, wherein said protein is a fusion protein. In another aspect is provided any of the methods disclosed herein, wherein the fusion protein is selected from etanercept, abatacept, and rilonacept. In another aspect is provided any of the methods disclosed herein, wherein the fusion protein is etanercept. In another aspect is provided any of the methods disclosed herein, wherein the fusion protein is abatacept. In another aspect is provided any of the methods disclosed herein, wherein the fusion protein is rilonacept.

In another aspect is provided a pharmaceutical composition comprising a first composition and a second composition for use as a medicament for the treatment of a condition or disease in a subject, including a skin condition or disease, wherein the (a) the first composition comprises a Spongilla; and (b) the second composition comprises one or more drugs in an amount effective to treat a skin disease or condition in a subject.

In another aspect is provided any of the pharmaceutical compositions comprising a first composition and a second composition for use as a medicament for the treatment of a condition or disease in a subject disclosed herein, including a skin condition or disease, wherein the first composition comprises Spongilla, and the second composition comprises a therapeutically effective amount of one or more drugs. In another aspect is provided any of the uses disclosed herein, wherein the one or more drugs is not a botulinum toxin, an antibiotic, an anti-inflammatory, an antiseptic, or an anesthetic. In another aspect is provided any of the methods disclosed herein, wherein the the one or more drugs is not a non-steroidal anti-inflammatory. In another aspect is provided any of the methods disclosed herein, wherein the the one or more drugs is not a steroidal anti-inflammatory. In another aspect is provided any of the methods disclosed herein, wherein the one or more drugs is not a botulinum toxin. In another aspect is provided any of the methods disclosed herein, wherein the one or more drugs is not an antibiotic. In another aspect is provided any of the methods disclosed herein, wherein the one or more drugs is not an anti-inflammatory. In another aspect is provided any of the methods disclosed herein, wherein the one or more drugs is not an antiseptic. In another aspect is provided any of the methods disclosed herein, wherein the one or more drugs is not an anesthetic.

In another aspect is provided any of the methods disclosed herein, wherein said drug is selected from a T-cell co-stimulation modulator, an antagonist of one or more interleukin receptors, an antagonist of one or more interleukins, an interferon-gamma antagonist, a tissue necrosis factor-alpha antagonist, and a transforming growth factor-beta agonist. In another aspect is provided any of the methods disclosed herein, wherein said drug is a T-cell co-stimulation modulator. In another aspect is provided any of the methods disclosed herein, wherein said drug is an antagonist of one or more interleukin receptors. In another aspect is provided any of the methods disclosed herein, wherein said drug is an antagonist of one or more interleukins. In another aspect is provided any of the methods disclosed herein, wherein said drug is an interferon-gamma antagonist. In another aspect is provided any of the methods disclosed herein, wherein said drug is a tissue necrosis factor-alpha antagonist. In another aspect is provided any of the methods disclosed herein, wherein said drug is a transforming growth factor-beta agonist.

In another aspect is provided any of the uses disclosed herein, wherein the disease or condition, including a skin disease or condition, is selected from plaque psoriasis, psoriatic arthritis, atopic dermatitis, acne vulgaris, acne rosacea type 1, acne rosacea type 2, psoriasis, hyperhidrosis, alopecia areata, androgenic alopecia, keloids, hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, melasma, rheumatoid arthritis, lichen planus, pityriasis rubra pilaris, ichthyosis, and palmoplantar pustulosis. In another aspect is provided any of the pharmaceutical compositions comprising a first composition and a second composition for use as a medicament for the treatment of a skin condition or disease in a subject disclosed herein, wherein the disease or condition is plaque psoriasis. In another aspect is provided any of the methods disclosed herein, wherein the disease or condition is psoriatic arthritis. In another aspect is provided any of the pharmaceutical compositions comprising a first composition and a second composition for use as a medicament for the treatment of a skin condition or disease in a subject disclosed herein, wherein the disease or condition is atopic dermatitis. In another aspect is provided any of the pharmaceutical compositions comprising a first composition and a second composition for use as a medicament for the treatment of a skin condition or disease in a subject disclosed herein, wherein the disease or condition is acne vulgaris. In another aspect is provided any of the pharmaceutical compositions comprising a first composition and a second composition for use as a medicament for the treatment of a skin condition or disease in a subject disclosed herein, wherein the disease or condition is acne rosacea type 1. In another aspect is provided any of the pharmaceutical compositions comprising a first composition and a second composition for use as a medicament for the treatment of a skin condition or disease in a subject disclosed herein, wherein the disease or condition is acne rosacea type 2. In another aspect is provided any of the pharmaceutical compositions comprising a first composition and a second composition for use as a medicament for the treatment of a skin condition or disease in a subject disclosed herein, wherein the disease or condition is psoriasis. In another aspect is provided any of the pharmaceutical compositions comprising a first composition and a second composition for use as a medicament for the treatment of a skin condition or disease in a subject disclosed herein, wherein the disease or condition is hyperhidrosis. In another aspect is provided any of the pharmaceutical compositions comprising a first composition and a second composition for use as a medicament for the treatment of a skin condition or disease in a subject disclosed herein, wherein the disease or condition is alopecia. In another aspect is provided any of the pharmaceutical compositions comprising a first composition and a second composition for use as a medicament for the treatment of a skin condition or disease in a subject disclosed herein, wherein the disease or condition is areata. In another aspect is provided any of the pharmaceutical compositions comprising a first composition and a second composition for use as a medicament for the treatment of a skin condition or disease in a subject disclosed herein, wherein the disease or condition is androgenic alopecia. In another aspect is provided any of the pharmaceutical compositions comprising a first composition and a second composition for use as a medicament for the treatment of a skin condition or disease in a subject disclosed herein, wherein the disease or condition is the treatment of keloids. In another aspect is provided any of the pharmaceutical compositions comprising a first composition and a second composition for use as a medicament for the treatment of a skin condition or disease in a subject disclosed herein, wherein the disease or condition is the treatment of hypertrophic scars. In another aspect is provided any of the pharmaceutical compositions comprising a first composition and a second composition for use as a medicament for the treatment of a skin condition or disease in a subject disclosed herein, wherein the disease or condition is hidradenitis suppurativa. In another aspect is provided any of the pharmaceutical compositions comprising a first composition and a second composition for use as a medicament for the treatment of a skin condition or disease in a subject disclosed herein, wherein the disease or condition is Raynaud phenomenon. In another aspect is provided any of the pharmaceutical compositions comprising a first composition and a second composition for use as a medicament for the treatment of a skin condition or disease in a subject disclosed herein, wherein the disease or condition is post-herpetic neuralgia. In another aspect is provided any of the pharmaceutical compositions comprising a first composition and a second composition for use as a medicament for the treatment of a skin condition or disease in a subject disclosed herein, wherein the disease or condition is Hailey-Hailey disease. In another aspect is provided any of the pharmaceutical compositions comprising a first composition and a second composition for use as a medicament for the treatment of a skin condition or disease in a subject disclosed herein, wherein the disease or condition is IgA bullous dermatosis. In another aspect is provided any of the pharmaceutical compositions comprising a first composition and a second composition for use as a medicament for the treatment of a skin condition or disease in a subject disclosed herein, wherein the disease or condition is epidermolysis bullosa Simplex Weber-Cockane. In another aspect is provided any of the pharmaceutical compositions comprising a first composition and a second composition for use as a medicament for the treatment of a skin condition or disease in a subject disclosed herein, wherein the disease or condition is Darier disease. In another aspect is provided any of the pharmaceutical compositions comprising a first composition and a second composition for use as a medicament for the treatment of a skin condition or disease in a subject disclosed herein, wherein the disease or condition is pachyonchia congenita. In another aspect is provided any of the pharmaceutical compositions comprising a first composition and a second composition for use as a medicament for the treatment of a skin condition or disease in a subject disclosed herein, wherein the disease or condition is aquagenic keratoderma. In another aspect is provided any of the pharmaceutical compositions comprising a first composition and a second composition for use as a medicament for the treatment of a skin condition or disease in a subject disclosed herein, wherein the disease or condition is notalgia paresthetic. In another aspect is provided any of the pharmaceutical compositions comprising a first composition and a second composition for use as a medicament for the treatment of a skin condition or disease in a subject disclosed herein, wherein the disease or condition is pompholyx (dyshidrotic eczema). In another aspect is provided any of the pharmaceutical compositions comprising a first composition and a second composition for use as a medicament for the treatment of a skin condition or disease in a subject disclosed herein, wherein the disease or condition is chromhidrosis and bromhidrosis. In another aspect is provided any of the pharmaceutical compositions comprising a first composition and a second composition for use as a medicament for the treatment of a skin condition or disease in a subject disclosed herein, wherein the disease or condition is eccrine nevus. In another aspect is provided any of the pharmaceutical compositions comprising a first composition and a second composition for use as a medicament for the treatment of a skin condition or disease in a subject disclosed herein, wherein the disease or condition is facial rhytides. In another aspect is provided any of the pharmaceutical compositions comprising a first composition and a second composition for use as a medicament for the treatment of a skin condition or disease in a subject disclosed herein, wherein the disease or condition is atrohpic acne scars. In another aspect is provided any of the pharmaceutical compositions comprising a first composition and a second composition for use as a medicament for the treatment of a skin condition or disease in a subject disclosed herein, wherein the disease or condition is melasma. In another aspect is provided any of the pharmaceutical compositions comprising a first composition and a second composition for use as a medicament for the treatment of a skin condition or disease in a subject disclosed herein, wherein the disease or condition is rheumatoid arthritis. In another aspect is provided any of the pharmaceutical compositions comprising a first composition and a second composition for use as a medicament for the treatment of a skin condition or disease in a subject disclosed herein, wherein the disease or condition is lichen planus. In another aspect is provided any of the pharmaceutical compositions comprising a first composition and a second composition for use as a medicament for the treatment of a skin condition or disease in a subject disclosed herein, wherein the disease or condition is pityriasis rubra pilaris. In another aspect is provided any of the pharmaceutical compositions comprising a first composition and a second composition for use as a medicament for the treatment of a skin condition or disease in a subject disclosed herein, wherein the disease or condition is ichthyosis. In another aspect is provided any of the pharmaceutical compositions comprising a first composition and a second composition for use as a medicament for the treatment of a skin condition or disease in a subject disclosed herein, wherein the disease or condition is palmoplantar pustulosis.

In another aspect is provided any of the pharmaceutical compositions comprising a first composition and a second composition for use as a medicament for the treatment of a condition or disease in a subject disclosed herein, including a skin condition or disease, wherein the one or more drugs is not a botulinum toxin, an antibiotic, an anti-inflammatory, an antiseptic, or an anesthetic. In another aspect is provided any of the pharmaceutical compositions comprising a first composition and a second composition for use as a medicament for the treatment of a skin condition or disease in a subject disclosed herein, wherein the anti-inflammatory is selected from non-steroidal anti-inflammatories and steroidal anti-inflammatories. In another aspect is provided any of the pharmaceutical compositions comprising a first composition and a second composition for use as a medicament for the treatment of a skin condition or disease in a subject disclosed herein, wherein the one or more drugs is not a botulinum toxin. In another aspect is provided any of the pharmaceutical compositions comprising a first composition and a second composition for use as a medicament for the treatment of a skin condition or disease in a subject disclosed herein, wherein the one or more drugs is not an antibiotic. In another aspect is provided any of the pharmaceutical compositions comprising a first composition and a second composition for use as a medicament for the treatment of a skin condition or disease in a subject disclosed herein, wherein the one or more drugs is not an anti-inflammatory. In another aspect is provided any of the pharmaceutical compositions comprising a first composition and a second composition for use as a medicament for the treatment of a skin condition or disease in a subject disclosed herein, wherein the one or more drugs is not an antiseptic. In another aspect is provided any of the pharmaceutical compositions comprising a first composition and a second composition for use as a medicament for the treatment of a skin condition or disease in a subject disclosed herein, wherein the one or more drugs is not an anesthetic.

In another aspect is provided any of the pharmaceutical compositions comprising a first composition and a second composition for use as a medicament for the treatment of a condition or disease in a subject disclosed herein, including a skin condition or disease, wherein the first composition comprises a Spongilla; and (b) the second composition comprises one or more drugs in an amount effective to treat a skin disease or condition in a subject, wherein said drug is selected from a T-cell co-stimulation modulator, an antagonist of one or more interleukin receptors, an antagonist of one or more interleukins, an interferon-gamma antagonist, a tissue necrosis factor-alpha antagonist, and a transforming growth factor-beta agonist. In another aspect is provided any of the pharmaceutical compositions comprising a first composition and a second composition for use as a medicament for the treatment of a skin condition or disease in a subject disclosed herein, wherein said drug is a T-cell co-stimulation modulator. In another aspect is provided any of the pharmaceutical compositions comprising a first composition and a second composition for use as a medicament for the treatment of a skin condition or disease in a subject disclosed herein, wherein said drug is an antagonist of one or more interleukin receptors. In another aspect is provided any of the pharmaceutical compositions comprising a first composition and a second composition for use as a medicament for the treatment of a skin condition or disease in a subject disclosed herein, wherein said drug is an antagonist of one or more interleukins. In another aspect is provided any of the pharmaceutical compositions comprising a first composition and a second composition for use as a medicament for the treatment of a skin condition or disease in a subject disclosed herein, wherein said drug is an interferon-gamma antagonist. In another aspect is provided any of the pharmaceutical compositions comprising a first composition and a second composition for use as a medicament for the treatment of a skin condition or disease in a subject disclosed herein, wherein said drug is a tissue necrosis factor-alpha antagonist. In another aspect is provided any of the pharmaceutical compositions comprising a first composition and a second composition for use as a medicament for the treatment of a skin condition or disease in a subject disclosed herein, wherein said drug is a transforming growth factor-beta agonist.

In another aspect is provided any of the pharmaceutical compositions comprising a first composition and a second composition for use as a medicament for the treatment of a condition or disease in a subject disclosed herein, including a skin condition or disease, the first composition comprises a Spongilla; and (b) the second composition comprises one or more drugs in an amount effective to treat a skin disease or condition in a subject, wherein said drug is an antagonist of one or more interleukin receptors. In another aspect is provided any of the pharmaceutical compositions comprising a first composition and a second composition for use as a medicament for the treatment of a skin condition or disease in a subject disclosed herein, wherein said interleukin receptor is IL-1R. In another aspect is provided any of the pharmaceutical compositions comprising a first composition and a second composition for use as a medicament for the treatment of a skin condition or disease in a subject disclosed herein, wherein said interleukin receptor is IL-1RA. In another aspect is provided any of the pharmaceutical compositions comprising a first composition and a second composition for use as a medicament for the treatment of a skin condition or disease in a subject disclosed herein, wherein said interleukin receptor is IL-1RB. In another aspect is provided any of the pharmaceutical compositions comprising a first composition and a second composition for use as a medicament for the treatment of a skin condition or disease in a subject disclosed herein, wherein said interleukin receptor is IL-2R. In another aspect is provided any of the pharmaceutical compositions comprising a first composition and a second composition for use as a medicament for the treatment of a skin condition or disease in a subject disclosed herein, wherein said interleukin receptor is IL-4R. In another aspect is provided any of the pharmaceutical compositions comprising a first composition and a second composition for use as a medicament for the treatment of a skin condition or disease in a subject disclosed herein, wherein said interleukin receptor is IL-5R. In another aspect is provided any of the pharmaceutical compositions comprising a first composition and a second composition for use as a medicament for the treatment of a skin condition or disease in a subject disclosed herein, wherein said interleukin receptor is IL-6R. In another aspect is provided any of the pharmaceutical compositions comprising a first composition and a second composition for use as a medicament for the treatment of a skin condition or disease in a subject disclosed herein, wherein said interleukin receptor is IL-10R. In another aspect is provided any of the pharmaceutical compositions comprising a first composition and a second composition for use as a medicament for the treatment of a skin condition or disease in a subject disclosed herein, wherein said interleukin receptor is IL-12R. In another aspect is provided any of the pharmaceutical compositions comprising a first composition and a second composition for use as a medicament for the treatment of a skin condition or disease in a subject disclosed herein, wherein said interleukin receptor is IL-13R. In another aspect is provided any of the pharmaceutical compositions comprising a first composition and a second composition for use as a medicament for the treatment of a skin condition or disease in a subject disclosed herein, wherein said interleukin receptor is IL-17R. In another aspect is provided any of the pharmaceutical compositions comprising a first composition and a second composition for use as a medicament for the treatment of a skin condition or disease in a subject disclosed herein, wherein said interleukin receptor is IL-17RA. In another aspect is provided any of the pharmaceutical compositions comprising a first composition and a second composition for use as a medicament for the treatment of a skin condition or disease in a subject disclosed herein, wherein said interleukin receptor is IL-21R. In another aspect is provided any of the pharmaceutical compositions comprising a first composition and a second composition for use as a medicament for the treatment of a skin condition or disease in a subject disclosed herein, wherein said interleukin receptor is IL-22R. In another aspect is provided any of the pharmaceutical compositions comprising a first composition and a second composition for use as a medicament for the treatment of a skin condition or disease in a subject disclosed herein, wherein said interleukin receptor is IL-23R. In another aspect is provided any of the pharmaceutical compositions comprising a first composition and a second composition for use as a medicament for the treatment of a skin condition or disease in a subject disclosed herein, wherein said interleukin receptor is IL-31R. In another aspect is provided any of the pharmaceutical compositions comprising a first composition and a second composition for use as a medicament for the treatment of a skin condition or disease in a subject disclosed herein, wherein said interleukin receptor is IL-35R.

In another aspect is provided any of the pharmaceutical compositions comprising a first composition and a second composition for use as a medicament for the treatment of a condition or disease in a subject disclosed herein, including a skin condition or disease, the first composition comprises a Spongilla; and (b) the second composition comprises one or more drugs in an amount effective to treat a skin disease or condition in a subject, wherein said drug is an antagonist of one or more interleukins. In another aspect is provided any of the pharmaceutical compositions comprising a first composition and a second composition for use as a medicament for the treatment of a skin condition or disease in a subject disclosed herein, wherein said interleukin is IL-1. In another aspect is provided any of the pharmaceutical compositions comprising a first composition and a second composition for use as a medicament for the treatment of a skin condition or disease in a subject disclosed herein, wherein said interleukin is IL-1A. In another aspect is provided any of the pharmaceutical compositions comprising a first composition and a second composition for use as a medicament for the treatment of a skin condition or disease in a subject disclosed herein, wherein said interleukin is IL-1B. In another aspect is provided any of the pharmaceutical compositions comprising a first composition and a second composition for use as a medicament for the treatment of a skin condition or disease in a subject disclosed herein, wherein said interleukin is IL-2. In another aspect is provided any of the pharmaceutical compositions comprising a first composition and a second composition for use as a medicament for the treatment of a skin condition or disease in a subject disclosed herein, wherein said interleukin is IL-4. In another aspect is provided any of the pharmaceutical compositions comprising a first composition and a second composition for use as a medicament for the treatment of a skin condition or disease in a subject disclosed herein, wherein said interleukin is IL-5. In another aspect is provided any of the pharmaceutical compositions comprising a first composition and a second composition for use as a medicament for the treatment of a skin condition or disease in a subject disclosed herein, wherein said interleukin is IL-6. In another aspect is provided any of the pharmaceutical compositions comprising a first composition and a second composition for use as a medicament for the treatment of a skin condition or disease in a subject disclosed herein, wherein said interleukin is IL-10. In another aspect is provided any of the pharmaceutical compositions comprising a first composition and a second composition for use as a medicament for the treatment of a skin condition or disease in a subject disclosed herein, wherein said interleukin is IL-12. In another aspect is provided any of the pharmaceutical compositions comprising a first composition and a second composition for use as a medicament for the treatment of a skin condition or disease in a subject disclosed herein, wherein said interleukin is IL-13. In another aspect is provided any of the pharmaceutical compositions comprising a first composition and a second composition for use as a medicament for the treatment of a skin condition or disease in a subject disclosed herein, wherein said interleukin is IL-17. In another aspect is provided any of the pharmaceutical compositions comprising a first composition and a second composition for use as a medicament for the treatment of a skin condition or disease in a subject disclosed herein, wherein said interleukin is IL-17A. In another aspect is provided any of the pharmaceutical compositions comprising a first composition and a second composition for use as a medicament for the treatment of a skin condition or disease in a subject disclosed herein, wherein said interleukin is IL-21. In another aspect is provided any of the pharmaceutical compositions comprising a first composition and a second composition for use as a medicament for the treatment of a skin condition or disease in a subject disclosed herein, wherein said interleukin is IL-22. In another aspect is provided any of the pharmaceutical compositions comprising a first composition and a second composition for use as a medicament for the treatment of a skin condition or disease in a subject disclosed herein, wherein said interleukin is IL-23. In another aspect is provided any of the pharmaceutical compositions comprising a first composition and a second composition for use as a medicament for the treatment of a skin condition or disease in a subject disclosed herein, wherein said interleukin is IL-31. In another aspect is provided any of the pharmaceutical compositions comprising a first composition and a second composition for use as a medicament for the treatment of a skin condition or disease in a subject disclosed herein, wherein said interleukin is IL-35.

In another aspect is provided any of the pharmaceutical compositions comprising a first composition and a second composition for use as a medicament for the treatment of a condition or disease in a subject disclosed herein, including a skin condition or disease, wherein the first composition comprises a Spongilla; and (b) the second composition comprises one or more drugs in an amount effective to treat a skin disease or condition in a subject, wherein the one or more drugs is selected from a chemical compound, a mixture of chemical compounds, a biological macromolecule, or an extract made from biological materials. In another aspect is provided any of the pharmaceutical compositions comprising a first composition and a second composition for use as a medicament for the treatment of a skin condition or disease in a subject disclosed herein, wherein said one or more drugs is a biological macromolecule. In another aspect is provided any of the pharmaceutical compositions comprising a first composition and a second composition for use as a medicament for the treatment of a skin condition or disease in a subject disclosed herein, wherein the one or more drugs is a chemical compound. In another aspect is provided any of the pharmaceutical compositions comprising a first composition and a second composition for use as a medicament for the treatment of a skin condition or disease in a subject disclosed herein, wherein the one or more drugs is a mixture of chemical compounds. In another aspect is provided any of the pharmaceutical compositions comprising a first composition and a second composition for use as a medicament for the treatment of a skin condition or disease in a subject disclosed herein, wherein the one or more drugs is a biological macromolecule. In another aspect is provided any of the pharmaceutical compositions comprising a first composition and a second composition for use as a medicament for the treatment of a skin condition or disease in a subject disclosed herein, wherein the one or more drugs is an extract made from biological materials.

In another aspect is provided any of the pharmaceutical compositions comprising a first composition and a second composition for use as a medicament for the treatment of a condition or disease in a subject disclosed herein, including a skin condition or disease, wherein the first composition comprises a Spongilla; and (b) the second composition comprises one or more drugs in an amount effective to treat a skin disease or condition in a subject wherein said one or more drugs is a biological macromolecule selected from a recombinant protein, a fusion protein, an antibody, a monoclonal antibody, a humanized monoclonal antibody, a bivalent antibody, an antibody fragment, an antibody-drug conjugate, a Fc fragment, a Fab fragment, a Fab′ fragment, a (Fab′)2 fragment, a Fv fragment, and a scFv fragment. In another aspect is provided any of the pharmaceutical compositions comprising a first composition and a second composition for use as a medicament for the treatment of a skin condition or disease in a subject disclosed herein, wherein the one or more biological macromolecules is a recombinant protein. In another aspect is provided any of the pharmaceutical compositions comprising a first composition and a second composition for use as a medicament for the treatment of a skin condition or disease in a subject disclosed herein, wherein the one or more biological macromolecules is a fusion protein. In another aspect is provided any of the pharmaceutical compositions comprising a first composition and a second composition for use as a medicament for the treatment of a skin condition or disease in a subject disclosed herein, wherein the one or more biological macromolecules is an antibody. In another aspect is provided any of the pharmaceutical compositions comprising a first composition and a second composition for use as a medicament for the treatment of a skin condition or disease in a subject disclosed herein, wherein the one or more biological macromolecules is a monoclonal antibody. In another aspect is provided any of the pharmaceutical compositions comprising a first composition and a second composition for use as a medicament for the treatment of a skin condition or disease in a subject disclosed herein, wherein the one or more biological macromolecules is a humanized monoclonal antibody. In another aspect is provided any of the pharmaceutical compositions comprising a first composition and a second composition for use as a medicament for the treatment of a skin condition or disease in a subject disclosed herein, wherein the one or more biological macromolecules is a bivalent antibody. In another aspect is provided any of the pharmaceutical compositions comprising a first composition and a second composition for use as a medicament for the treatment of a skin condition or disease in a subject disclosed herein, wherein the one or more biological macromolecules is an antibody fragment. In another aspect is provided any of the pharmaceutical compositions comprising a first composition and a second composition for use as a medicament for the treatment of a skin condition or disease in a subject disclosed herein, wherein the one or more biological macromolecules is an antibody-drug conjugate. In another aspect is provided any of the pharmaceutical compositions comprising a first composition and a second composition for use as a medicament for the treatment of a skin condition or disease in a subject disclosed herein, wherein the one or more biological macromolecules is a Fc fragment. In another aspect is provided any of the pharmaceutical compositions comprising a first composition and a second composition for use as a medicament for the treatment of a skin condition or disease in a subject disclosed herein, wherein the one or more biological macromolecules is a Fab fragment. In another aspect is provided any of the pharmaceutical compositions comprising a first composition and a second composition for use as a medicament for the treatment of a skin condition or disease in a subject disclosed herein, wherein the one or more biological macromolecules is a Fab′ fragment. In another aspect is provided any of the pharmaceutical compositions comprising a first composition and a second composition for use as a medicament for the treatment of a skin condition or disease in a subject disclosed herein, wherein the one or more biological macromolecules is a (Fab′)2 fragment. In another aspect is provided any of the pharmaceutical compositions comprising a first composition and a second composition for use as a medicament for the treatment of a skin condition or disease in a subject disclosed herein, wherein the one or more biological macromolecules is a Fv fragment. In another aspect is provided any of the pharmaceutical compositions comprising a first composition and a second composition for use as a medicament for the treatment of a skin condition or disease in a subject disclosed herein, wherein the one or more biological macromolecules is a scFv fragment.

In another aspect is provided any of the pharmaceutical compositions comprising a first composition and a second composition for use as a medicament for the treatment of a condition or disease in a subject disclosed herein, including a skin condition or disease, wherein the first composition comprises a Spongilla; and (b) the second composition comprises one or more drugs in an amount effective to treat a skin disease or condition in a subject, wherein said one or more biological molecules is an antibody. In another aspect is provided any of the pharmaceutical compositions comprising a first composition and a second composition for use as a medicament for the treatment of a skin condition or disease in a subject disclosed herein, wherein said antibody is a monoclonal antibody. In another aspect is provided any of the pharmaceutical compositions comprising a first composition and a second composition for use as a medicament for the treatment of a skin condition or disease in a subject disclosed herein, wherein said monoclonal antibody is a humanized antibody. In another aspect is provided any of the pharmaceutical compositions comprising a first composition and a second composition for use as a medicament for the treatment of a skin condition or disease in a subject disclosed herein, wherein the antibody is selected from one or more subclasses consisting of IgG1, IgG2, and IgG4. In another aspect is provided any of the pharmaceutical compositions comprising a first composition and a second composition for use as a medicament for the treatment of a skin condition or disease in a subject disclosed herein, wherein said antibody is selected from one or more subclasses consisting of IgG1, IgG1/kappa, IgG1/lambda, IgG2, IgG2/kappa, and IgG4. In another aspect is provided any of the pharmaceutical compositions comprising a first composition and a second composition for use as a medicament for the treatment of a skin condition or disease in a subject disclosed herein, wherein the antibody is an IgG1 antibody. In another aspect is provided any of the pharmaceutical compositions comprising a first composition and a second composition for use as a medicament for the treatment of a skin condition or disease in a subject disclosed herein, wherein the antibody is an IgG2 antibody. In another aspect is provided any of the pharmaceutical compositions comprising a first composition and a second composition for use as a medicament for the treatment of a skin condition or disease in a subject disclosed herein, wherein the antibody is an IgG4 antibody. In another aspect is provided any of the pharmaceutical compositions comprising a first composition and a second composition for use as a medicament for the treatment of a skin condition or disease in a subject disclosed herein, wherein said antibody is an IgG1/kappa antibody. In another aspect is provided any of the pharmaceutical compositions comprising a first composition and a second composition for use as a medicament for the treatment of a skin condition or disease in a subject disclosed herein, wherein said antibody is an IgG1/lambda antibody. In another aspect is provided any of the pharmaceutical compositions comprising a first composition and a second composition for use as a medicament for the treatment of a skin condition or disease in a subject disclosed herein, wherein said antibody is an IgG2 antibody. In another aspect is provided any of the pharmaceutical compositions comprising a first composition and a second composition for use as a medicament for the treatment of a skin condition or disease in a subject disclosed herein, wherein said antibody is an IgG2/kappa antibody. In another aspect is provided any of the pharmaceutical compositions comprising a first composition and a second composition for use as a medicament for the treatment of a skin condition or disease in a subject disclosed herein, wherein said antibody an IgG4 antibody.

In another aspect is provided any of the pharmaceutical compositions comprising a first composition and a second composition for use as a medicament for the treatment of a condition or disease in a subject disclosed herein, including a skin condition or disease, the first composition comprises a Spongilla; and (b) the second composition comprises one or more drugs in an amount effective to treat a skin disease or condition in a subject, wherein said drug is an antibody and is selected from secukinumab, ixekizumab, adalimumab, brodalumab, guselkumab, ustekinumab, sarilumab, dupilumab, tildrakizumab, lebrikizumab, mepolizumab, fezakinumab, nemolizumab, risankizumab, BMS 981164, CMJ112, tralokinumab, cemiplimab, infliximab, basiliximab, daclizumab, efalizumab, ustekinumab, certolizumab pegol, ABX-IL8, omalizumab, mirikizumab, and MSB0010841 (ALX-0761). In another aspect is provided any of the pharmaceutical compositions comprising a first composition and a second composition for use as a medicament for the treatment of a skin condition or disease in a subject disclosed herein, wherein the antibody is secukinumab. In another aspect is provided any of the pharmaceutical compositions comprising a first composition and a second composition for use as a medicament for the treatment of a skin condition or disease in a subject disclosed herein, wherein the antibody is ixekizumab. In another aspect is provided any of the pharmaceutical compositions comprising a first composition and a second composition for use as a medicament for the treatment of a skin condition or disease in a subject disclosed herein, wherein the antibody is adalimumab. In another aspect is provided any of the pharmaceutical compositions comprising a first composition and a second composition for use as a medicament for the treatment of a skin condition or disease in a subject disclosed herein, wherein the antibody is brodalumab. In another aspect is provided any of the pharmaceutical compositions comprising a first composition and a second composition for use as a medicament for the treatment of a skin condition or disease in a subject disclosed herein, wherein the antibody is guselkumab. In another aspect is provided any of the pharmaceutical compositions comprising a first composition and a second composition for use as a medicament for the treatment of a skin condition or disease in a subject disclosed herein, wherein the antibody is ustekinumab. In another aspect is provided any of the pharmaceutical compositions comprising a first composition and a second composition for use as a medicament for the treatment of a skin condition or disease in a subject disclosed herein, wherein the antibody is sarilumab. In another aspect is provided any of the pharmaceutical compositions comprising a first composition and a second composition for use as a medicament for the treatment of a skin condition or disease in a subject disclosed herein, wherein the antibody is dupilumab. In another aspect is provided any of the pharmaceutical compositions comprising a first composition and a second composition for use as a medicament for the treatment of a skin condition or disease in a subject disclosed herein, wherein the antibody is tildrakizumab. In another aspect is provided any of the pharmaceutical compositions comprising a first composition and a second composition for use as a medicament for the treatment of a skin condition or disease in a subject disclosed herein, wherein the antibody is lebrikizumab. In another aspect is provided any of the pharmaceutical compositions comprising a first composition and a second composition for use as a medicament for the treatment of a skin condition or disease in a subject disclosed herein, wherein the antibody is mepolizumab. In another aspect is provided any of the pharmaceutical compositions comprising a first composition and a second composition for use as a medicament for the treatment of a skin condition or disease in a subject disclosed herein, wherein the antibody is fezakinumab. In another aspect is provided any of the pharmaceutical compositions comprising a first composition and a second composition for use as a medicament for the treatment of a skin condition or disease in a subject disclosed herein, wherein the antibody is nemolizumab. In another aspect is provided any of the pharmaceutical compositions comprising a first composition and a second composition for use as a medicament for the treatment of a skin condition or disease in a subject disclosed herein, wherein the antibody is risankizumab. In another aspect is provided any of the pharmaceutical compositions comprising a first composition and a second composition for use as a medicament for the treatment of a skin condition or disease in a subject disclosed herein, wherein the antibody is BMS 981164. In another aspect is provided any of the pharmaceutical compositions comprising a first composition and a second composition for use as a medicament for the treatment of a skin condition or disease in a subject disclosed herein, wherein the antibody is CMJ112. In another aspect is provided any of the pharmaceutical compositions comprising a first composition and a second composition for use as a medicament for the treatment of a skin condition or disease in a subject disclosed herein, wherein the antibody is tralokinumab. In another aspect is provided any of the pharmaceutical compositions comprising a first composition and a second composition for use as a medicament for the treatment of a skin condition or disease in a subject disclosed herein, wherein the antibody is cemiplimab. In another aspect is provided any of the pharmaceutical compositions comprising a first composition and a second composition for use as a medicament for the treatment of a skin condition or disease in a subject disclosed herein, wherein the antibody is infliximab. In another aspect is provided any of the pharmaceutical compositions comprising a first composition and a second composition for use as a medicament for the treatment of a skin condition or disease in a subject disclosed herein, wherein the antibody is basiliximab. In another aspect is provided any of the pharmaceutical compositions comprising a first composition and a second composition for use as a medicament for the treatment of a skin condition or disease in a subject disclosed herein, wherein the antibody is daclizumab. In another aspect is provided any of the pharmaceutical compositions comprising a first composition and a second composition for use as a medicament for the treatment of a skin condition or disease in a subject disclosed herein, wherein the antibody is efalizumab. In another aspect is provided any of the pharmaceutical compositions comprising a first composition and a second composition for use as a medicament for the treatment of a skin condition or disease in a subject disclosed herein, wherein the antibody is ustekinumab. In another aspect is provided any of the pharmaceutical compositions comprising a first composition and a second composition for use as a medicament for the treatment of a skin condition or disease in a subject disclosed herein, wherein the antibody is certolizumab pegol. In another aspect is provided any of the pharmaceutical compositions comprising a first composition and a second composition for use as a medicament for the treatment of a skin condition or disease in a subject disclosed herein, wherein the antibody is ABX-IL8. In another aspect is provided any of the pharmaceutical compositions comprising a first composition and a second composition for use as a medicament for the treatment of a skin condition or disease in a subject disclosed herein, wherein the antibody is omalizumab. In another aspect is provided any of the pharmaceutical compositions comprising a first composition and a second composition for use as a medicament for the treatment of a skin condition or disease in a subject disclosed herein, wherein the antibody is mirikizumab. In another aspect is provided any of the pharmaceutical compositions comprising a first composition and a second composition for use as a medicament for the treatment of a skin condition or disease in a subject disclosed herein, wherein the antibody is MSB0010841 (ALX-0761).

In another aspect is provided any of the pharmaceutical compositions comprising a first composition and a second composition for use as a medicament for the treatment of a condition or disease, including a skin condition in a subject disclosed herein, the first composition comprises a Spongilla; and (b) the second composition comprises one or more drugs in an amount effective to treat a skin disease or condition in a subject, wherein said drug is a biologic macromolecules that is a protein. In another aspect is provided any of the pharmaceutical compositions comprising a first composition and a second composition for use as a medicament for the treatment of a skin condition or disease in a subject disclosed herein, wherein said protein is selected from etanercept, abatacept, rilonacept, and anakinra. In another aspect is provided any of the pharmaceutical compositions comprising a first composition and a second composition for use as a medicament for the treatment of a skin condition or disease in a subject disclosed herein, wherein the protein is etanercept. In another aspect is provided any of the pharmaceutical compositions comprising a first composition and a second composition for use as a medicament for the treatment of a skin condition or disease in a subject disclosed herein, wherein the protein is abatacept. In another aspect is provided any of the pharmaceutical compositions comprising a first composition and a second composition for use as a medicament for the treatment of a skin condition or disease in a subject disclosed herein, wherein the protein is rilonacept. In another aspect is provided any of the pharmaceutical compositions comprising a first composition and a second composition for use as a medicament for the treatment of a skin condition or disease in a subject disclosed herein, wherein the protein is anakinra.

In another aspect is provided any of the pharmaceutical compositions comprising a first composition and a second composition for use as a medicament for the treatment of a condition or disease in a subject disclosed herein, including a skin condition or disease, the first composition comprises a Spongilla; and (b) the second composition comprises one or more drugs in an amount effective to treat a skin disease or condition in a subject, wherein the drug is fusion protein. In another aspect is provided any of the pharmaceutical compositions comprising a first composition and a second composition for use as a medicament for the treatment of a skin condition or disease in a subject disclosed herein, wherein the fusion protein is selected from etanercept, abatacept, and rilonacept. In another aspect is provided any of the pharmaceutical compositions comprising a first composition and a second composition for use as a medicament for the treatment of a skin condition or disease in a subject disclosed herein, wherein the fusion protein is etanercept. In another aspect is provided any of the pharmaceutical compositions comprising a first composition and a second composition for use as a medicament for the treatment of a skin condition or disease in a subject disclosed herein, wherein the fusion protein is abatacept. In another aspect is provided any of the pharmaceutical compositions comprising a first composition and a second composition for use as a medicament for the treatment of a skin condition or disease in a subject disclosed herein, wherein the fusion protein is rilonacept.

In one aspect is provided a drug product comprising a first composition and a second composition, wherein said first composition comprises Spongilla, and said second composition comprises one or more drugs in an amount effective to treat a disease or condition in a subject, including a skin condition or disease. In another aspect is provided a kit comprising (a) a first composition comprising Spongilla, and (b) a second composition comprising one or more drugs in an amount effective to treat a condition in a subject, including a skin condition. In another aspect is provided a drug product comprising a first composition and a second composition, wherein said first composition comprises Spongilla, and said second composition comprises one or more drugs in an amount effective to treat a skin disease or condition in a subject, wherein the second composition is in the form of a solution, an aqueous solution, a powder, or a gel.

In another aspect is provided any of the drug products or kits disclosed herein, wherein the disease or condition, including a skin disease or condition, is selected from plaque psoriasis, psoriatic arthritis, atopic dermatitis, acne vulgaris, acne rosacea type 1, acne rosacea type 2, psoriasis, hyperhidrosis, alopecia areata, androgenic alopecia, keloids, hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, melasma, rheumatoid arthritis, lichen planus, pityriasis rubra pilaris, ichthyosis, and palmoplantar pustulosis. In another aspect is provided any of the drug products or kits disclosed herein, wherein the disease or condition is plaque psoriasis. In another aspect is provided any of the methods disclosed herein, wherein the disease or condition is psoriatic arthritis. In another aspect is provided any of the drug products or kits disclosed herein, wherein the disease or condition is atopic dermatitis. In another aspect is provided any of the drug products or kits disclosed herein, wherein the disease or condition is acne vulgaris. In another aspect is provided any of the drug products or kits disclosed herein, wherein the disease or condition is acne rosacea type 1. In another aspect is provided any of the drug products or kits disclosed herein, wherein the disease or condition is acne rosacea type 2. In another aspect is provided any of the drug products or kits disclosed herein, wherein the disease or condition is psoriasis. In another aspect is provided any of the drug products or kits disclosed herein, wherein the disease or condition is hyperhidrosis. In another aspect is provided any of the drug products or kits disclosed herein, wherein the disease or condition is alopecia. In another aspect is provided any of the drug products or kits disclosed herein, wherein the disease or condition is areata. In another aspect is provided any of the drug products or kits disclosed herein, wherein the disease or condition is androgenic alopecia. In another aspect is provided any of the drug products or kits disclosed herein, wherein the disease or condition is the treatment of keloids. In another aspect is provided any of the drug products or kits disclosed herein, wherein the disease or condition is the treatment of hypertrophic scars. In another aspect is provided any of the drug products or kits disclosed herein, wherein the disease or condition is hidradenitis suppurativa. In another aspect is provided any of the drug products or kits disclosed herein, wherein the disease or condition is Raynaud phenomenon. In another aspect is provided any of the drug products or kits disclosed herein, wherein the disease or condition is post-herpetic neuralgia. In another aspect is provided any of the drug products or kits disclosed herein, wherein the disease or condition is Hailey-Hailey disease. In another aspect is provided any of the drug products or kits disclosed herein, wherein the disease or condition is IgA bullous dermatosis. In another aspect is provided any of the drug products or kits disclosed herein, wherein the disease or condition is epidermolysis bullosa Simplex Weber-Cockane. In another aspect is provided any of the drug products or kits disclosed herein, wherein the disease or condition is Darier disease. In another aspect is provided any of the drug products or kits disclosed herein, wherein the disease or condition is pachyonchia congenita. In another aspect is provided any of the drug products or kits disclosed herein, wherein the disease or condition is aquagenic keratoderma. In another aspect is provided any of the drug products or kits disclosed herein, wherein the disease or condition is notalgia paresthetic. In another aspect is provided any of the drug products or kits disclosed herein, wherein the disease or condition is pompholyx (dyshidrotic eczema). In another aspect is provided any of the drug products or kits disclosed herein, wherein the disease or condition is chromhidrosis and bromhidrosis. In another aspect is provided any of the drug products or kits disclosed herein, wherein the disease or condition is eccrine nevus. In another aspect is provided any of the drug products or kits disclosed herein, wherein the disease or condition is facial rhytides. In another aspect is provided any of the drug products or kits disclosed herein, wherein the disease or condition is atrohpic acne scars. In another aspect is provided any of the drug products or kits disclosed herein, wherein the disease or condition is melasma. In another aspect is provided any of the drug products or kits disclosed herein, wherein the disease or condition is rheumatoid arthritis. In another aspect is provided any of the drug products or kits disclosed herein, wherein the disease or condition is lichen planus. In another aspect is provided any of the drug products or kits disclosed herein, wherein the disease or condition is pityriasis rubra pilaris. In another aspect is provided any of the drug products or kits disclosed herein, wherein the disease or condition is ichthyosis. In another aspect is provided any of the drug products or kits disclosed herein, wherein the disease or condition is palmoplantar pustulosis.

In another aspect is provided any of the drug products or kits disclosed herein, wherein the one or more drugs is not a botulinum toxin, an antibiotic, an anti-inflammatory, an antiseptic, or an anesthetic. In another aspect is provided any of the drug products or kits disclosed herein, wherein the anti-inflammatory is selected from non-steroidal anti-inflammatories and steroidal anti-inflammatories. In another aspect is provided any of the drug products or kits disclosed herein, wherein the one or more drugs is not a botulinum toxin. In another aspect is provided any of the drug products or kits disclosed herein, wherein the one or more drugs is not an antibiotic. In another aspect is provided any of the drug products or kits disclosed herein, wherein the one or more drugs is not an anti-inflammatory. In another aspect is provided any of the drug products or kits disclosed herein, wherein the one or more drugs is not an antiseptic. In another aspect is provided any of the drug products or kits disclosed herein, wherein the one or more drugs is not an anesthetic.

In another aspect is provided any of the drug products or kits disclosed herein, wherein said drug is selected from a T-cell co-stimulation modulator, an antagonist of one or more interleukin receptors, an antagonist of one or more interleukins, an interferon-gamma antagonist, a tissue necrosis factor-alpha antagonist, and a transforming growth factor-beta agonist. In another aspect is provided any of the drug products or kits disclosed herein, wherein said drug is a T-cell co-stimulation modulator. In another aspect is provided any of the drug products or kits disclosed herein, wherein said drug is an antagonist of one or more interleukin receptors. In another aspect is provided any of the drug products or kits disclosed herein, wherein said drug is an antagonist of one or more interleukins. In another aspect is provided any of the drug products or kits disclosed herein, wherein said drug is an interferon-gamma antagonist. In another aspect is provided any of the drug products or kits disclosed herein, wherein said drug is a tissue necrosis factor-alpha antagonist. In another aspect is provided any of the drug products or kits disclosed herein, wherein said drug is a transforming growth factor-beta agonist.

In another aspect is provided any of the drug products or kits disclosed herein, wherein said drug is an antagonist of one or more interleukin receptors. In another aspect is provided any of the drug products or kits disclosed herein, wherein said interleukin receptor is IL-1R. In another aspect is provided any of the drug products or kits disclosed herein, wherein said interleukin receptor is IL-1RA. In another aspect is provided any of the drug products or kits disclosed herein, wherein said interleukin receptor is IL-1RB. In another aspect is provided any of the drug products or kits disclosed herein, wherein said interleukin receptor is IL-2R. In another aspect is provided any of the drug products or kits disclosed herein, wherein said interleukin receptor is IL-4R. In another aspect is provided any of the drug products or kits disclosed herein, wherein said interleukin receptor is IL-5R. In another aspect is provided any of the drug products or kits disclosed herein, wherein said interleukin receptor is IL-6R. In another aspect is provided any of the drug products or kits disclosed herein, wherein said interleukin receptor is IL-10R. In another aspect is provided any of the drug products or kits disclosed herein, wherein said interleukin receptor is IL-12R. In another aspect is provided any of the drug products or kits disclosed herein, wherein said interleukin receptor is IL-13R. In another aspect is provided any of the drug products or kits disclosed herein, wherein said interleukin receptor is IL-17R. In another aspect is provided any of the drug products or kits disclosed herein, wherein said interleukin receptor is IL-17RA. In another aspect is provided any of the drug products or kits disclosed herein, wherein said interleukin receptor is IL-21R. In another aspect is provided any of the drug products or kits disclosed herein, wherein said interleukin receptor is IL-22R. In another aspect is provided any of the drug products or kits disclosed herein, wherein said interleukin receptor is IL-23R. In another aspect is provided any of the drug products or kits disclosed herein, wherein said interleukin receptor is IL-31R. In another aspect is provided any of the drug products or kits disclosed herein, wherein said interleukin receptor is IL-35R.

In another aspect is provided any of the drug products or kits disclosed herein, wherein said drug is an antagonist of one or more interleukins. In another aspect is provided any of the drug products or kits disclosed herein, wherein said interleukin is IL-1. In another aspect is provided any of the drug products or kits disclosed herein, wherein said interleukin is IL-1A. In another aspect is provided any of the drug products or kits disclosed herein, wherein said interleukin is IL-1B. In another aspect is provided any of the drug products or kits disclosed herein, wherein said interleukin is IL-2. In another aspect is provided any of the drug products or kits disclosed herein, wherein said interleukin is IL-4. In another aspect is provided any of the drug products or kits disclosed herein, wherein said interleukin is IL-5. In another aspect is provided any of the drug products or kits disclosed herein, wherein said interleukin is IL-6. In another aspect is provided any of the drug products or kits disclosed herein, wherein said interleukin is IL-10. In another aspect is provided any of the drug products or kits disclosed herein, wherein said interleukin is IL-12. In another aspect is provided any of the drug products or kits disclosed herein, wherein said interleukin is IL-13. In another aspect is provided any of the drug products or kits disclosed herein, wherein said interleukin is IL-17. In another aspect is provided any of the drug products or kits disclosed herein, wherein said interleukin is IL-17A. In another aspect is provided any of the drug products or kits disclosed herein, wherein said interleukin is IL-21. In another aspect is provided any of the drug products or kits disclosed herein, wherein said interleukin is IL-22. In another aspect is provided any of the drug products or kits disclosed herein, wherein said interleukin is IL-23. In another aspect is provided any of the drug products or kits disclosed herein, wherein said interleukin is IL-31. In another aspect is provided any of the drug products or kits disclosed herein, wherein said interleukin is IL-35.

In another aspect is provided any of the drug products or kits disclosed herein, wherein the one or more drugs is selected from a chemical compound, a mixture of chemical compounds, a biological macromolecule, or an extract made from biological materials. In another aspect is provided any of the drug products or kits disclosed herein, wherein said one or more drugs is a biological macromolecule. In another aspect is provided any of the drug products or kits disclosed herein, wherein the one or more drugs is a chemical compound. In another aspect is provided any of the drug products or kits disclosed herein, wherein the one or more drugs is a mixture of chemical compounds. In another aspect is provided any of the drug products or kits disclosed herein, wherein the one or more drugs is a biological macromolecule. In another aspect is provided any of the drug products or kits disclosed herein, wherein the one or more drugs is an extract made from biological materials.

In another aspect is provided any of the drug products or kits disclosed herein, wherein said one or more biological macromolecules is selected from a recombinant protein, a fusion protein, an antibody, a monoclonal antibody, a humanized monoclonal antibody, a bivalent antibody, an antibody fragment, an antibody-drug conjugate, a Fc fragment, a Fab fragment, a Fab′ fragment, a (Fab′)2 fragment, a Fv fragment, and a scFv fragment. In another aspect is provided any of the drug products or kits disclosed herein, wherein the one or more biological macromolecules is a recombinant protein. In another aspect is provided any of the drug products or kits disclosed herein, wherein the one or more biological macromolecules is a fusion protein. In another aspect is provided any of the drug products or kits disclosed herein, wherein the one or more biological macromolecules is an antibody. In another aspect is provided any of the drug products or kits disclosed herein, wherein the one or more biological macromolecules is a monoclonal antibody. In another aspect is provided any of the drug products or kits disclosed herein, wherein the one or more biological macromolecules is a humanized monoclonal antibody. In another aspect is provided any of the drug products or kits disclosed herein, wherein the one or more biological macromolecules is a bivalent antibody. In another aspect is provided any of the drug products or kits disclosed herein, wherein the one or more biological macromolecules is an antibody fragment. In another aspect is provided any of the drug products or kits disclosed herein, wherein the one or more biological macromolecules is an antibody-drug conjugate. In another aspect is provided any of the drug products or kits disclosed herein, wherein the one or more biological macromolecules is a Fc fragment. In another aspect is provided any of the drug products or kits disclosed herein, wherein the one or more biological macromolecules is a Fab fragment. In another aspect is provided any of the drug products or kits disclosed herein, wherein the one or more biological macromolecules is a Fab′ fragment. In another aspect is provided any of the drug products or kits disclosed herein, wherein the one or more biological macromolecules is a (Fab′)2 fragment. In another aspect is provided any of the drug products or kits disclosed herein, wherein the one or more biological macromolecules is a Fv fragment. In another aspect is provided any of the drug products or kits disclosed herein, wherein the one or more biological macromolecules is a scFv fragment.

In another aspect is provided any of the drug products or kits disclosed herein, wherein said one or more biological molecules is an antibody. In another aspect is provided any of the drug products or kits disclosed herein, wherein said antibody is a monoclonal antibody. In another aspect is provided any of the drug products or kits disclosed herein, wherein said monoclonal antibody is a humanized antibody. In another aspect is provided any of the drug products or kits disclosed herein, wherein the antibody is selected from one or more subclasses consisting of IgG1, IgG2, and IgG4. In another aspect is provided any of the drug products or kits disclosed herein, wherein said antibody is selected from one or more subclasses consisting of IgG1, IgG1/kappa, IgG1/lambda, IgG2, IgG2/kappa, and IgG4. In another aspect is provided any of the drug products or kits disclosed herein, wherein the antibody is an IgG1 antibody. . In another aspect is provided any of the drug products or kits disclosed herein, wherein the antibody is an IgG2 antibody. In another aspect is provided any of the drug products or kits disclosed herein, wherein the antibody is an IgG4 antibody. In another aspect is provided any of the drug products or kits disclosed herein, wherein said antibody is an IgG1/kappa antibody. In another aspect is provided any of the drug products or kits disclosed herein, wherein said antibody is an IgG1/lambda antibody. In another aspect is provided any of the drug products or kits disclosed herein, wherein said antibody is an IgG2 antibody. In another aspect is provided any of the drug products or kits disclosed herein, wherein said antibody is an IgG2/kappa antibody. In another aspect is provided any of the drug products or kits disclosed herein, wherein said antibody an IgG4 antibody.

In another aspect is provided any of the drug products or kits disclosed herein, wherein said antibody is selected from secukinumab, ixekizumab, adalimumab, brodalumab, guselkumab, ustekinumab, sarilumab, dupilumab, tildrakizumab, lebrikizumab, mepolizumab, fezakinumab, nemolizumab, risankizumab, BMS 981164, CMJ112, tralokinumab, cemiplimab, infliximab, basiliximab, daclizumab, efalizumab, ustekinumab, certolizumab pegol, ABX-IL8, omalizumab, mirikizumab, and MSB0010841 (ALX-0761). In another aspect is provided any of the drug products or kits disclosed herein, wherein the antibody is secukinumab. In another aspect is provided any of the drug products or kits disclosed herein, wherein the antibody is ixekizumab. In another aspect is provided any of the drug products or kits disclosed herein, wherein the antibody is adalimumab. In another aspect is provided any of the drug products or kits disclosed herein, wherein the antibody is brodalumab. In another aspect is provided any of the drug products or kits disclosed herein, wherein the antibody is guselkumab. In another aspect is provided any of the drug products or kits disclosed herein, wherein the antibody is ustekinumab. In another aspect is provided any of the drug products or kits disclosed herein, wherein the antibody is sarilumab. In another aspect is provided any of the drug products or kits disclosed herein, wherein the antibody is dupilumab. In another aspect is provided any of the drug products or kits disclosed herein, wherein the antibody is tildrakizumab. In another aspect is provided any of the drug products or kits disclosed herein, wherein the antibody is lebrikizumab. In another aspect is provided any of the drug products or kits disclosed herein, wherein the antibody is mepolizumab. In another aspect is provided any of the drug products or kits disclosed herein, wherein the antibody is fezakinumab. In another aspect is provided any of the drug products or kits disclosed herein, wherein the antibody is nemolizumab. In another aspect is provided any of the drug products or kits disclosed herein, wherein the antibody is risankizumab. In another aspect is provided any of the drug products or kits disclosed herein, wherein the antibody is BMS 981164. In another aspect is provided any of the drug products or kits disclosed herein, wherein the antibody is CMJ112. In another aspect is provided any of the drug products or kits disclosed herein, wherein the antibody is tralokinumab. In another aspect is provided any of the drug products or kits disclosed herein, wherein the antibody is cemiplimab. In another aspect is provided any of the drug products or kits disclosed herein, wherein the antibody is infliximab. In another aspect is provided any of the drug products or kits disclosed herein, wherein the antibody is basiliximab. In another aspect is provided any of the drug products or kits disclosed herein, wherein the antibody is daclizumab. In another aspect is provided any of the drug products or kits disclosed herein, wherein the antibody is efalizumab. In another aspect is provided any of the drug products or kits disclosed herein, wherein the antibody is ustekinumab. In another aspect is provided any of the drug products or kits disclosed herein, wherein the antibody is certolizumab pegol. In another aspect is provided any of the drug products or kits disclosed herein, wherein the antibody is ABX-IL8. In another aspect is provided any of the drug products or kits disclosed herein, wherein the antibody is omalizumab. In another aspect is provided any of the drug products or kits disclosed herein, wherein the antibody is mirikizumab. In another aspect is provided any of the drug products or kits disclosed herein, wherein the antibody is MSB0010841 (ALX-0761)

In another aspect is provided any of the drug products or kits disclosed herein, wherein said biologic macromolecules is a protein. In another aspect is provided any of the drug products or kits disclosed herein, wherein said protein is selected from etanercept, abatacept, rilonacept, and anakinra. In another aspect is provided any of the drug products or kits disclosed herein, wherein the protein is etanercept. In another aspect is provided any of the drug products or kits disclosed herein, wherein the protein is abatacept. In another aspect is provided any of the drug products or kits disclosed herein, wherein the protein is rilonacept. In another aspect is provided any of the drug products or kits disclosed herein, wherein the protein is anakinra.

In another aspect is provided any of the drug products or kits disclosed herein, wherein said protein is a fusion protein. In another aspect is provided any of the drug products or kits disclosed herein, wherein the fusion protein is selected from etanercept, abatacept, and rilonacept. In another aspect is provided any of the drug products or kits disclosed herein, wherein the fusion protein is etanercept. In another aspect is provided any of the drug products or kits disclosed herein, wherein the fusion protein is abatacept. In another aspect is provided any of the drug products or kits disclosed herein, wherein the fusion protein is rilonacept.

Spongilla, including Spongilla lacustris, and powders prepared from Spongilla that are utilized in the methods, uses, compositions for use as a medicament, kits and drug products disclosed herein may be obtained, processed and characterized by methods known to those having ordinary skill in the art. For example, U.S. Pat. No. 7,604,821 describes the harvest, processing and characterization of several species of Spongilla, including Spongilla lacustris. The disclosure of U.S. Pat. No. 7,604,821 is incorporated herein by reference in its entirety. Sponge materials may be collected using methods commonly known to those skilled in the art of marine biology. For example, sponges can be collected manually using basic under water diving techniques, or in deeper waters larger colonies are harvested using the Agassiz trawl (AGT) or epibenthic sledge (EBS). Under certain environmental conditions, Spongilla colonies occur in a thin crust-like carpet several meters across and must be collected manually, with fork-like tools, and nets. The collected sponge mass is dried, cleaned of gross contamination, such as shells, stems, plants, rocks and other impurities, and is then washed to remove dirt, sand, silt and soluble impurities. The cleaned sponge mass is weighed and dried using methods known to those of ordinary skill in the art, such as air drying and the use of dryers that are used to dehydrate foods and pharmaceuticals. The sponge mass is dried until residual moisture content is less than a desired value as further disclosed herein. Residual moisture measurements can be performed using methods commonly known in the arts of food sciences, analytical chemistry or the pharmaceutical sciences. For example, 10 grams of dried material may be placed on a tared weighing boat and then weighed. The weighed material is then exposed to a heat source such as a drying oven or heat lamp operated at an appropriate temperature, the sample is then cooled in a desiccated chamber and re-weighed. Residual moisture is calculated as the percent difference between the sample weight before drying and the weight after cooling. Following drying, the sponge materials may be packaged in sealed containers, which optionally protect the materials from light, moisture and oxygen. The materials may then be further tested for the presence of pathogens, coliform organisms and organisms that represent a bioburden. The materials may be further heated or irradiated, as disclosed herein, to reduce any pathogens, coliform organisms or other organisms that represent a bioburden. The materials may then be further processed using methods known to those having ordinary skill in the art to provide a powder comprising particles having a desired size. For example, the sponge materials may be ground, and the resulting materials passed through one or more sieves of a defined size to provide a resulting material comprising particles having a uniform, or substantially uniform size. After final processing and sizing processes are completed, the dried sponge material may be packaged in airtight moisture-proof containers and stored at an appropriate temperature, such as at about room or ambient temperature.

In another aspect is provided any of the methods, compositions for use as a medicament, drug products and kits disclosed herein, wherein the first composition comprises Spongilla in the form of a powder. Materials comprising Spongilla may be prepared in powdered form having particles of substantially the same size, using techniques known to those having ordinary skill in the art, such as grinding and sieving. In another aspect is provided any of the methods, compositions for use as a medicament, drug products and kits disclosed herein, wherein the Spongilla is in the form of a powder comprising particles that are substantially uniform in size. In another aspect is provided any of the methods, compositions for use as a medicament, drug products and kits disclosed herein, wherein not less than about 50% of the particles comprising the Spongilla powder pass through a US 70-mesh screen. In another aspect is provided any of the methods, compositions for use as a medicament, drug products and kits disclosed herein, wherein not less than about 60%, or about 70%, or about 75%, or about 80%, or about 85%, or about 90%, or about 95%, or about 96%, or about 97%, or about 98%, or about 99% of the particles comprising the Spongilla powder pass through a US 70-mesh screen. In another aspect is provided any of the methods, compositions for use as a medicament, drug products and kits disclosed herein, wherein not less than about 95%, or about 96%, or about 97%, or about 98%, or about 99% of the particles comprising the Spongilla powder pass through a US 70-mesh screen. In another aspect is provided any of the methods, compositions for use as a medicament, drug products and kits disclosed herein, wherein not less than about 95% of the particles comprising the Spongilla powder pass through a US 70-mesh screen. In another aspect is provided any of the methods, compositions for use as a medicament, drug products and kits disclosed herein, wherein not less than about 96% of the particles comprising the Spongilla powder pass through a US 70-mesh screen. In another aspect is provided any of the methods, compositions for use as a medicament, drug products and kits disclosed herein, wherein not less than about 97% of the particles comprising the Spongilla powder pass through a US 70-mesh screen. In another aspect is provided any of the methods, compositions for use as a medicament, drug products and kits disclosed herein, wherein not less than about 98% of the particles comprising the Spongilla powder pass through a US 70-mesh screen. In another aspect is provided any of the methods, compositions for use as a medicament, drug products and kits disclosed herein, wherein not less than about 99% of the particles comprising the Spongilla powder pass through a US 70-mesh screen. The particles of Spongilla may be manufactured or produced from Spongilla materials that are harvested by procedures known to those of ordinary skill in the art, such as determining the appropriate harvest period, removal of foreign materials, drying, milling and grinding using equipment known to those of ordinary skill in the art.

In another aspect is provided any of the methods, compositions for use as a medicament, drug products and kits disclosed herein, wherein the particles comprising the Spongilla powder have an average length of from about 50 μm to about 500 μm. In another aspect is provided any of the methods, compositions for use as a medicament, drug products and kits disclosed herein, wherein the particles comprising the Spongilla powder have an average length of from about 50 μm to about 400 μm, or from about 50 μm to about 350 μm, or from about 50 μm to about 300 μm, or from about 50 μm to about 250 μm, or from about 50 μm to about 200 μm, or from about 75 μm to about 500 μm, or from about 75 μm to about 450 μm, or from about 80 μm to about 450 μm, or from about 80 μm to about 400 μm, or from about 85 μm to about 450 μm, or from about 85 μm to about 400 μm, or from about 90 μm to about 450 μm, or from about 90 μm to about 400 μm, or from about 90 μm to about 350 μm, or from about 100 μm to about 450 μm, or from about 100 μm to about 400 μm, or from about 100 μm to about 350 μm, or from about 100 μm to about 300 μm, or from about 100 μm to about 250 μm, or from about 100 μm to about 200 μm, or from about 150 μm to about 500 μm, or from about 100 μm to about 450 μm, or from about 150 μm to about 400 μm, or from about 150 μm to about 350 μm, or from about 150 μm to about 350 μm, or from about 150 μm to about 300 μm, or from about 150 μm to about 250 μm, or from about 150 μm to about 200 μm, or from about 175 μm to about 450 μm, or from about 175 μm to about 400 μm, or from about 175 μm to about 350 μm, or from about 175 μm to about 300 μm, or from about 175 μm to about 250 μm, or from about 175 μm to about 200 μm. In another aspect is provided any of the methods, compositions for use as a medicament, drug products and kits disclosed herein, wherein the particles comprising the Spongilla powder have an average length of about 50 μm, or about 75 μm, or about 80 μm, or about 85 μm, or about 90 μm, or about 100 μm, or about 125 μm, or about 150 μm, or about 175 μm, or about 200 μm, or about 225 μm, or about 250 μm, or about 300 μm, or about 350 μm, or about 400 μm, or about 450 μm, or about 500 μm. In another aspect is provided any of the methods, compositions for use as a medicament, drug products and kits disclosed herein, wherein the particles comprising the Spongilla powder have an average length of about 200 μm. The particles comprising the Spongilla powder may be manufactured or produced from Spongilla materials that are harvested by procedures known to those of ordinary skill in the art, such as milling and grinding using equipment known to those of ordinary skill in the art. The average length of particles comprising the Spongilla powder may be measured using analytical methods known to those of ordinary skill in the art, such as, for example, scanning electron microscopy (SEM) and sieve analysis. Sieve analysis may also be used to determine the particle size distribution of the particles comprising the Spongilla powder.

In another aspect is provided any of the methods, compositions for use as a medicament, drug products and kits disclosed herein, wherein the particles comprising the Spongilla powder have an average diameter of from about 5 μm to about 50 μm. In another aspect is provided any of the methods, compositions for use as a medicament, drug products and kits disclosed herein, wherein the particles comprising the Spongilla powder have an average diameter of from about 5 μm to about 45 μm, or from about 5 μm to about 40 μm, from about 5 μm to about 35 μm, from about 5 μm to about 30 μm, from about 5 μm to about 25 μm, from about 5 μm to about 20 μm, from about 10 μm to about 45 μm, from about 10 μm to about 40 μm, from about 10 μm to about 35 μm, from about 10 μm to about 30 μm, from about 10 μm to about 25 μm, from about 10 μm to about 20 μm. In another aspect is provided any of the methods, compositions for use as a medicament, drug products and kits disclosed herein, wherein the particles comprising the Spongilla powder have an average diameter of about 5 μm, or about 10 μm, or about 15 μm, or about 20 μm, or about 25 μm, or about 30 μm, or about 35 μm, or about 40 μm, or about 45 μm, or about 50 μm. The particles comprising the Spongilla powder may be manufactured or produced from Spongilla materials that are harvested by procedures known to those of ordinary skill in the art, such as milling and grinding using equipment known to those of ordinary skill in the art. The average diameter of particles comprising the Spongilla powder may be measured using analytical methods known to those of ordinary skill in the art, such as, for example, scanning electron microscopy (SEM) and sieve analysis. Sieve analysis may also be used to determine the particle size distribution of the particles comprising the Spongilla powder.

In another aspect is provided any of the methods, compositions for use as a medicament, drug products and kits disclosed herein, wherein the particles comprising the Spongilla powder have an aspect ratio of from about 1 to about 100. In another aspect is provided any of the methods, compositions for use as a medicament, drug products and kits disclosed herein, wherein the particles comprising the Spongilla powder have an aspect ratio of from about 1 to about 75, or from about 1 to about 50, or from about 1 to about 25, or from about 1 to about 20, or from about 1 to about 15, or from about 5 to about 100, or from about 5 to about 75, or from about 5 to about 50, or from about 5 to about 40, or from about 5 to about 35, or from about 5 to about 30, or from about 5 to about 25, or from about 5 to about 20, or from about 5 to about 15, or from about 7 to about 50, or from about 7 to about 45, or from about 7 to about 40, or from about 7 to about 35, or from about 7 to about 30, or from about 7 to about 25, or from about 10 to about 50, or from about 10 to about 45, or from about 10 to about 40, or from about 10 to about 35, or from about 10 to about 30, or from about 10 to about 25, or from about 10 to about 15. In another aspect is provided any of the methods, compositions for use as a medicament, drug products and kits disclosed herein, wherein the particles comprising the Spongilla powder have an aspect ratio of about 5, or about 6, or about 7, or about 8, or about 9, or about 10, or about 11, or about 12, or about 13, or about 14, or about 15, or about 16, or about 17, or about 18, or about 19, or about 20, or about 21, or about 22, or about 23, or about 24, or about 25, or about 26, or about 27, or about 28, or about 29, or about 30, or about 35, or about 40, or about 45, or about 50, or about 75, or about 100. The particles comprising the Spongilla powder may be manufactured or produced from Spongilla materials that are harvested by procedures known to those of ordinary skill in the art, such as milling and grinding using equipment known to those of ordinary skill in the art. The aspect ratio of particles comprising the Spongilla powder may be measured using analytical methods known to those of ordinary skill in the art, such as, for example, scanning electron microscopy (SEM) and sieve analysis. Sieve analysis may also be used to determine the particle size distribution of the particles comprising the Spongilla powder.

Materials comprising Spongilla may be processed and dried, using techniques known to those having ordinary skill in the art, such as the use of drying ovens, to provide materials having a desired residual moisture content. In another aspect is provided any of the methods, compositions for use as a medicament, drug products and kits disclosed herein, wherein the first composition comprising Spongilla has a residual moisture content of not more than about 20%. In another aspect is provided any of the methods, compositions for use as a medicament, drug products and kits disclosed herein, wherein the first composition has a residual moisture content of not more than about 15%, or not more than about 10%, or not more than about 9%, or not more than about 8%, or not more than about 7%, or not more than about 6%, or not more than about 5%, or not more than about 4%, or not more than about 3%, or not more than about 2%, or not more than 1%. In another aspect is provided any of the methods, compositions for use as a medicament, drug products and kits disclosed herein, wherein the first composition has a residual moisture content of not more than about 5%. In another aspect is provided any of the methods, compositions for use as a medicament, drug products and kits disclosed herein, wherein the first composition has a residual moisture content of not more than about 4%. In another aspect is provided any of the methods, compositions for use as a medicament, drug products and kits disclosed herein, wherein the first composition has a residual moisture content of not more than about 3%. In another aspect is provided any of the methods, compositions for use as a medicament, drug products and kits disclosed herein, wherein the first composition has a residual moisture content of not more than about 2%. In another aspect is provided any of the methods, compositions for use as a medicament, drug products and kits disclosed herein, wherein the first composition has a residual moisture content of not more than about 1%. The moisture content of the Spongilla materials can be reduced by heating the raw Spongilla materials using methods known to those of ordinary skill in the art, such as by open-air drying, or by use of a conventional oven dryer or a vacuum dryer, using equipment known to those of ordinary skill in the art. For example, raw Spongilla materials may be placed into a tray and heated in a drying oven at a temperature range from about 30° C. to about 200° C., for example to about 70° C., for a period of time necessary to reduce the residual moisture content to the desired level. The level of residual moisture of the materials may be measured using methods known to those of ordinary skill in the art such as those described in the United States Pharmacopeia methods USP <731> (Loss on Drying) and USP <921> (Water Determination).

Materials comprising Spongilla may be treated in order to reduce the bioburden, such as aerobic and anaerobic microbes, yeast and mold, Coliform bacteria, Salmonella, Pseudomonas aeruginosa, and Staphylococcus aureus, of the materials prior to their packaging and use, such as by use of heat treatment or irradiation, such as the use of gamma irradiation.

In another aspect is provided any of the methods, compositions for use as a medicament, drug products and kits disclosed herein, wherein the first composition has a combined aerobic and anaerobic microbial content of not more than about 25×10⁴ colony-forming units per gram (CFU/g). In another aspect is provided any of the methods, compositions for use as a medicament, drug products and kits disclosed herein, wherein the first composition has a combined aerobic and anaerobic microbial content of not more than about 10×10⁴ CFU/g, or not more than about 5×10⁴ CFU/g, or not more than about 1×10⁴ CFU/g, or not more than about 5×10³ CFU/g, or not more than about 1×10⁴ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods, compositions for use as a medicament, drug products and kits disclosed herein, wherein the first composition has a combined aerobic and anaerobic microbial content of not more than about 1,000 CFU/g. In another aspect is provided any of the methods, compositions for use as a medicament, drug products and kits disclosed herein, wherein the first composition has a combined aerobic and anaerobic microbial content of not more than about 750 CFU/g. In another aspect is provided any of the methods, compositions for use as a medicament, drug products and kits disclosed herein, wherein the first composition has a combined aerobic and anaerobic microbial content of not more than about 500 CFU/g. In another aspect is provided any of the methods, compositions for use as a medicament, drug products and kits disclosed herein, wherein the first composition has a combined aerobic and anaerobic microbial content of not more than about 250 CFU/g. In another aspect is provided any of the methods, compositions for use as a medicament, drug products and kits disclosed herein, wherein the first composition has a combined aerobic and anaerobic microbial content of not more than about 200 CFU/g. In another aspect is provided any of the methods, compositions for use as a medicament, drug products and kits disclosed herein, wherein the first composition has a combined aerobic and anaerobic microbial content of not more than about 150 CFU/g. In another aspect is provided any of the methods, compositions for use as a medicament, drug products and kits disclosed herein, wherein the first composition has a combined aerobic and anaerobic microbial content of not more than about 100 CFU/g. In another aspect is provided any of the methods, compositions for use as a medicament, drug products and kits disclosed herein, wherein the first composition has a combined aerobic and anaerobic microbial content of not more than about 75 CFU/g. In another aspect is provided any of the methods, compositions for use as a medicament, drug products and kits disclosed herein, wherein the first composition has a combined aerobic and anaerobic microbial content of not more than about 50 CFU/g. In another aspect is provided any of the methods, compositions for use as a medicament, drug products and kits disclosed herein, wherein the first composition has a combined aerobic and anaerobic microbial content of not more than about 25 CFU/g. In another aspect is provided any of the methods, compositions for use as a medicament, drug products and kits disclosed herein, wherein the first composition has a combined aerobic and anaerobic microbial content of not more than about 20 CFU/g. In another aspect is provided any of the methods, compositions for use as a medicament, drug products and kits disclosed herein, wherein the first composition has a combined aerobic and anaerobic microbial content of not more than about 10 CFU/g. In another aspect is provided any of the methods, compositions for use as a medicament, drug products and kits disclosed herein, wherein the first composition has a combined aerobic and anaerobic microbial content of not more than about 1 CFU/g. The combined aerobic and anaerobic microbial content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The combined aerobic and anaerobic microbial content of the Spongilla materials may be measured by methods known to those of ordinary skill in the art, such as those described in the United States Pharmacopeia method USP <61> (Microbial Enumeration Tests).

In another aspect is provided any of the methods, compositions for use as a medicament, drug products and kits disclosed herein, wherein the first composition comprising Spongilla has a combined yeast and mold content of not more than about 25×10⁴ colony-forming units per gram (CFU/g). In another aspect is provided any of the methods, compositions for use as a medicament, drug products and kits disclosed herein, wherein the first composition has a combined yeast and mold content of not more than about or not more than about 5×10⁴ CFU/g, or not more than about 1×10⁴ CFU/g, or not more than about 5×10³ CFU/g, or not more than about 1×10⁴ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods, compositions for use as a medicament, drug products and kits disclosed herein, wherein the first composition has a combined yeast and mold content of not more than about 1,000 CFU/g. In another aspect is provided any of the methods, compositions for use as a medicament, drug products and kits disclosed herein, wherein the first composition has a combined yeast and mold content of not more than about 750 CFU/g. In another aspect is provided any of the methods, compositions for use as a medicament, drug products and kits disclosed herein, wherein the first composition has a combined yeast and mold content of not more than about 500 CFU/g. In another aspect is provided any of the methods, compositions for use as a medicament, drug products and kits disclosed herein, wherein the first composition has a combined yeast and mold content of not more than about 250 CFU/g. In another aspect is provided any of the methods, compositions for use as a medicament, drug products and kits disclosed herein, wherein the first composition has a combined yeast and mold content of not more than about 200 CFU/g. In another aspect is provided any of the methods, compositions for use as a medicament, drug products and kits disclosed herein, wherein the first composition has a combined yeast and mold content of not more than about 150 CFU/g. In another aspect is provided any of the methods, compositions for use as a medicament, drug products and kits disclosed herein, wherein the first composition has a combined yeast and mold content of not more than about 100 CFU/g. In another aspect is provided any of the methods, compositions for use as a medicament, drug products and kits disclosed herein, wherein the first composition has a combined yeast and mold content of not more than about 75 CFU/g. In another aspect is provided any of the methods, compositions for use as a medicament, drug products and kits disclosed herein, wherein the first composition has a combined yeast and mold content of not more than about 50 CFU/g. In another aspect is provided any of the methods, compositions for use as a medicament, drug products and kits disclosed herein, wherein the first composition has a combined yeast and mold content of not more than about 25 CFU/g. In another aspect is provided any of the methods, compositions for use as a medicament, drug products and kits disclosed herein, wherein the first composition has a combined yeast and mold content of not more than about 20 CFU/g. In another aspect is provided any of the methods, compositions for use as a medicament, drug products and kits disclosed herein, wherein the first composition has a combined yeast and mold content of not more than about 10 CFU/g. In another aspect is provided any of the methods, compositions for use as a medicament, drug products and kits disclosed herein, wherein the first composition has a combined yeast and mold content of not more than about 1 CFU/g. The combined yeast and mold content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The combined yeast and mold content of the Spongilla materials may be measured by methods known to those of ordinary skill in the art, such as those described in the United States Pharmacopeia method USP <61> (Microbial Enumeration Tests).

In another aspect is provided any of the methods, compositions for use as a medicament, drug products and kits disclosed herein, wherein the amount of Coliform bacteria in the first composition is not more than about 25×10⁴ colony-forming units per gram (CFU/g). In another aspect is provided any of the methods, compositions for use as a medicament, drug products and kits disclosed herein, wherein the amount of Coliform bacteria in the first composition is not more than about 5×10⁴ CFU/g, or not more than about 1×10⁴ CFU/g, or not more than about 5×10³ CFU/g, or not more than about 1×10⁴ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods, compositions for use as a medicament, drug products and kits disclosed herein, wherein the first composition has a Coliform bacteria content of not more than about 1,000 CFU/g. In another aspect is provided any of the methods, compositions for use as a medicament, drug products and kits disclosed herein, wherein the first composition has a Coliform bacteria content of not more than about 750 CFU/g. In another aspect is provided any of the methods, compositions for use as a medicament, drug products and kits disclosed herein, wherein the first composition has a Coliform bacteria content of not more than about 500 CFU/g. In another aspect is provided any of the methods, compositions for use as a medicament, drug products and kits disclosed herein, wherein the first composition has a Coliform bacteria content of not more than about 250 CFU/g. In another aspect is provided any of the methods, compositions for use as a medicament, drug products and kits disclosed herein, wherein the first composition has a Coliform bacteria content of not more than about 200 CFU/g. In another aspect is provided any of the methods, compositions for use as a medicament, drug products and kits disclosed herein, wherein the first composition has a Coliform bacteria content of not more than about 150 CFU/g. In another aspect is provided any of the methods, compositions for use as a medicament, drug products and kits disclosed herein, wherein the first composition has a Coliform bacteria content of not more than about 100 CFU/g. In another aspect is provided any of the methods, compositions for use as a medicament, drug products and kits disclosed herein, wherein the first composition has a Coliform bacteria content of not more than about 75 CFU/g. In another aspect is provided any of the methods, compositions for use as a medicament, drug products and kits disclosed herein, wherein the first composition has a Coliform bacteria content of not more than about 50 CFU/g. In another aspect is provided any of the methods, compositions for use as a medicament, drug products and kits disclosed herein, wherein the first composition has a Coliform bacteria content of not more than about 25 CFU/g. In another aspect is provided any of the methods, compositions for use as a medicament, drug products and kits disclosed herein, wherein the first composition has a Coliform bacteria content of not more than about 20 CFU/g. In another aspect is provided any of the methods, compositions for use as a medicament, drug products and kits disclosed herein, wherein the first composition has a Coliform bacteria content of not more than about 10 CFU/g. In another aspect is provided any of the methods, compositions for use as a medicament, drug products and kits disclosed herein, wherein the first composition has a Coliform bacteria content of not more than about 1 CFU/g. In another aspect is provided any of the methods, compositions for use as a medicament, drug products and kits disclosed herein, wherein the first composition has no detectable Coliform bacterial content. The Coliform bacteria content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The Coliform bacteria content of the Spongilla materials may be measured by methods known to those of ordinary skill in the art, such as those described in the United States Pharmacopeia method USP <62> (Tests for Specified Microorganisms).

In another aspect is provided any of the methods, compositions for use as a medicament, drug products and kits disclosed herein, wherein the amount of Salmonella in the first composition is not more than about 25×10⁴ colony-forming units per gram (CFU/g). In another aspect is provided any of the methods, compositions for use as a medicament, drug products and kits disclosed herein, wherein the amount of Salmonella in the first composition is not more than about 5×10⁴ CFU/g, or not more than about 1×10⁴ CFU/g, or not more than about 5×10³ CFU/g, or not more than about 1×10⁴ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods, compositions for use as a medicament, drug products and kits disclosed herein, wherein the first composition has a Salmonella content of not more than about 1,000 CFU/g. In another aspect is provided any of the methods, compositions for use as a medicament, drug products and kits disclosed herein, wherein the first composition has a Salmonella content of not more than about 750 CFU/g. In another aspect is provided any of the methods, compositions for use as a medicament, drug products and kits disclosed herein, wherein the first composition has a Salmonella content of not more than about 500 CFU/g. In another aspect is provided any of the methods, compositions for use as a medicament, drug products and kits disclosed herein, wherein the first composition has a Salmonella content of not more than about 250 CFU/g. In another aspect is provided any of the methods, compositions for use as a medicament, drug products and kits disclosed herein, wherein the first composition has a Salmonella content of not more than about 200 CFU/g. In another aspect is provided any of the methods, compositions for use as a medicament, drug products and kits disclosed herein, wherein the first composition has a Salmonella content of not more than about 150 CFU/g. In another aspect is provided any of the methods, compositions for use as a medicament, drug products and kits disclosed herein, wherein the first composition has a Salmonella content of not more than about 100 CFU/g. In another aspect is provided any of the methods, compositions for use as a medicament, drug products and kits disclosed herein, wherein the first composition has a Salmonella content of not more than about 75 CFU/g. In another aspect is provided any of the methods, compositions for use as a medicament, drug products and kits disclosed herein, wherein the first composition has a Salmonella content of not more than about 50 CFU/g. In another aspect is provided any of the methods, compositions for use as a medicament, drug products and kits disclosed herein, wherein the first composition has a Salmonella content of not more than about 25 CFU/g. In another aspect is provided any of the methods, compositions for use as a medicament, drug products and kits disclosed herein, wherein the first composition has a Salmonella content of not more than about 20 CFU/g. In another aspect is provided any of the methods, compositions for use as a medicament, drug products and kits disclosed herein, wherein the first composition has a Salmonella content of not more than about 10 CFU/g. In another aspect is provided any of the methods, compositions for use as a medicament, drug products and kits disclosed herein, wherein the first composition has a Salmonella content of not more than about 1 CFU/g. In another aspect is provided any of the methods, compositions for use as a medicament, drug products and kits disclosed herein, wherein the first composition has no detectable Salmonella content. The Salmonella content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The Salmonella content of the Spongilla materials may be measured by methods known to those of ordinary skill in the art, such as those described in the United States Pharmacopeia method USP <62> (Tests for Specified Microorganisms).

In another aspect is provided any of the methods, compositions for use as a medicament, drug products and kits disclosed herein, wherein the amount of Pseudomonas aeruginosa bacteria in the first composition is not more than about 25×10⁴ colony-forming units per gram (CFU/g). In another aspect is provided any of the methods, compositions for use as a medicament, drug products and kits disclosed herein, wherein the amount of Pseudomonas aeruginosa bacteria in the first composition is not more than about 5×10⁴ CFU/g, or not more than about 1×10⁴ CFU/g, or not more than about 5×10³ CFU/g, or not more than about 1×10⁴ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods, compositions for use as a medicament, drug products and kits disclosed herein, wherein the first composition has a Pseudomonas aeruginosa bacteria content of not more than about 1,000 CFU/g. In another aspect is provided any of the methods, compositions for use as a medicament, drug products and kits disclosed herein, wherein the first composition has a Pseudomonas aeruginosa bacteria content of not more than about 750 CFU/g. In another aspect is provided any of the methods, compositions for use as a medicament, drug products and kits disclosed herein, wherein the first composition has a Pseudomonas aeruginosa bacteria content of not more than about 500 CFU/g. In another aspect is provided any of the methods, compositions for use as a medicament, drug products and kits disclosed herein, wherein the first composition has a Pseudomonas aeruginosa bacteria content of not more than about 250 CFU/g. In another aspect is provided any of the methods, compositions for use as a medicament, drug products and kits disclosed herein, wherein the first composition has a Pseudomonas aeruginosa bacteria content of not more than about 200 CFU/g. In another aspect is provided any of the methods, compositions for use as a medicament, drug products and kits disclosed herein, wherein the first composition has a Pseudomonas aeruginosa bacteria content of not more than about 150 CFU/g. In another aspect is provided any of the methods, compositions for use as a medicament, drug products and kits disclosed herein, wherein the first composition has a Pseudomonas aeruginosa bacteria content of not more than about 100 CFU/g. In another aspect is provided any of the methods, compositions for use as a medicament, drug products and kits disclosed herein, wherein the first composition has a Pseudomonas aeruginosa bacteria content of not more than about 75 CFU/g. In another aspect is provided any of the methods, compositions for use as a medicament, drug products and kits disclosed herein, wherein the first composition has a Pseudomonas aeruginosa bacteria content of not more than about 50 CFU/g. In another aspect is provided any of the methods, compositions for use as a medicament, drug products and kits disclosed herein, wherein the first composition has a Pseudomonas aeruginosa bacteria content of not more than about 25 CFU/g. In another aspect is provided any of the methods, compositions for use as a medicament, drug products and kits disclosed herein, wherein the first composition has a Pseudomonas aeruginosa bacteria content of not more than about 20 CFU/g. In another aspect is provided any of the methods, compositions for use as a medicament, drug products and kits disclosed herein, wherein the first composition has a Pseudomonas aeruginosa bacteria content of not more than about 10 CFU/g. In another aspect is provided any of the methods, compositions for use as a medicament, drug products and kits disclosed herein, wherein the first composition has a Pseudomonas aeruginosa bacteria content of not more than about 1 CFU/g. In another aspect is provided any of the methods, compositions for use as a medicament, drug products and kits disclosed herein, wherein the first composition has no detectable Pseudomonas aeruginosa bacteria content. The Pseudomonas aeruginosa bacteria content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The Pseudomonas aeruginosa bacteria content of the Spongilla materials may be measured by methods known to those of ordinary skill in the art, such as those described in the United States Pharmacopeia method USP <62> (Tests for Specified Microorganisms).

In another aspect is provided any of the methods, compositions for use as a medicament, drug products and kits disclosed herein, wherein the amount of Staphylococcus aureus bacteria in the first composition is not more than about 25×10⁴ colony-forming units per gram (CFU/g). In another aspect is provided any of the methods, compositions for use as a medicament, drug products and kits disclosed herein, wherein the amount of Staphylococcus aureus bacteria in the first composition is not more than about 5×10⁴ CFU/g, or not more than about 1×10⁴ CFU/g, or not more than about 5×10³ CFU/g, or not more than about 1×10⁴ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods, compositions for use as a medicament, drug products and kits disclosed herein, wherein the first composition has a Staphylococcus aureus bacteria content of not more than about 1,000 CFU/g. In another aspect is provided any of the methods, compositions for use as a medicament, drug products and kits disclosed herein, wherein the first composition has a Staphylococcus aureus bacteria content of not more than about 750 CFU/g. In another aspect is provided any of the methods, compositions for use as a medicament, drug products and kits disclosed herein, wherein the first composition has a Staphylococcus aureus bacteria content of not more than about 500 CFU/g. In another aspect is provided any of the methods, compositions for use as a medicament, drug products and kits disclosed herein, wherein the first composition has a Staphylococcus aureus bacteria content of not more than about 250 CFU/g. In another aspect is provided any of the methods, compositions for use as a medicament, drug products and kits disclosed herein, wherein the first composition has a Staphylococcus aureus bacteria content of not more than about 200 CFU/g. In another aspect is provided any of the methods, compositions for use as a medicament, drug products and kits disclosed herein, wherein the first composition has a Staphylococcus aureus bacteria content of not more than about 150 CFU/g. In another aspect is provided any of the methods, compositions for use as a medicament, drug products and kits disclosed herein, wherein the first composition has a Staphylococcus aureus bacteria content of not more than about 100 CFU/g. In another aspect is provided any of the methods, compositions for use as a medicament, drug products and kits disclosed herein, wherein the first composition has a Staphylococcus aureus bacteria content of not more than about 75 CFU/g. In another aspect is provided any of the methods, compositions for use as a medicament, drug products and kits disclosed herein, wherein the first composition has a Staphylococcus aureus bacteria content of not more than about 50 CFU/g. In another aspect is provided any of the methods, compositions for use as a medicament, drug products and kits disclosed herein, wherein the first composition has a Staphylococcus aureus bacteria content of not more than about 25 CFU/g. In another aspect is provided any of the methods, compositions for use as a medicament, drug products and kits disclosed herein, wherein the first composition has a Staphylococcus aureus bacteria content of not more than about 20 CFU/g. In another aspect is provided any of the methods, compositions for use as a medicament, drug products and kits disclosed herein, wherein the first composition has a Staphylococcus aureus bacteria content of not more than about 10 CFU/g. In another aspect is provided any of the methods, compositions for use as a medicament, drug products and kits disclosed herein, wherein the first composition has a Staphylococcus aureus bacteria content of not more than about 1 CFU/g. In another aspect is provided any of the methods, compositions for use as a medicament, drug products and kits disclosed herein, wherein the first composition has no detectable Staphylococcus aureus bacteria content. The Staphylococcus aureus bacteria content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The Staphylococcus aureus bacteria content of the Spongilla materials may be measured by methods known to those of ordinary skill in the art, such as those described in the United States Pharmacopeia method USP <62> (Tests for Specified Microorganisms).

In another aspect is provided any of the methods, compositions for use as a medicament, drug products and kits disclosed herein, wherein the first composition is packaged prior to use. In another aspect is provided any of the methods, compositions for use as a medicament, drug products and kits disclosed herein, wherein the first composition is prepared by heating to at least about 70° C. prior to being packaged in order to reduce the bioburden. In another aspect is provided any of the methods, compositions for use as a medicament, drug products and kits disclosed herein, wherein the first composition is prepared by heating to at least about 50° C., or at least about 60° C., or at least about 75° C., or at least about 80° C., or at least about 85° C., or at least about 90° C., or at least about 100° C., or at least about 110° C., or at least about 115° C., or at least about 120° C., or at least about 125° C., or at least about 130° C., or at least about 135° C., or at least about 140° C., or at least about 150° C., or at least about 160° C., or at least about 170° C., or at least about 180° C., or at least about 190° C., or at least about 200° C. prior to being packaged.

In another aspect is provided any of the methods, compositions for use as a medicament, drug products and kits disclosed herein, wherein the first composition comprising Spongilla is heated to at least about 70° C. for at least about 5 minutes prior being packaged. In another aspect is provided any of the methods, compositions for use as a medicament, drug products and kits disclosed herein, wherein the first composition is heated to at least about 70° C. for at least about 10 minutes, or at least about 15 minutes, or at least about 20 minutes, or at least about 25 minutes, or at least about 30 minutes, or at least about 35 minutes, or at least about 40 minutes, or at least about 45 minutes, or at least about 50 minutes, or at least about 55 minutes, or at least about 60 minutes, or at least about 75 minutes, or at least about 90 minutes, or at least about 120 minutes, or at least about 180 minutes, or at least about 4 hours, or at least about 5 hours, or at least about 6 hours, or at least about 7 hours, or at least about 8 hours, or at least about 9 hours, or at least about 10 hours, or at least about 11 hours, or at least about 12 hours, or at least about 24 hours prior being packaged.

In another aspect is provided any of the methods, compositions for use as a medicament, drug products and kits disclosed herein, wherein the first composition comprising Spongilla is prepared by treating with ionizing radiation, such as gamma radiation, prior to being packaged or after packaging. For example, gamma irradiation may be performed on the raw Spongilla material prior to grinding to reduce the particle size, following grinding to reduce the particle size, the materials packaged in bulk and or the materials following packaging in unit dose containers. The materials may be treated with ionizing radiation, such as gamma radiation, using methods and equipment known to those of ordinary skill in the art, such as gamma irradiators or electron beam irradiators. In another aspect is provided any of the methods, compositions for use as a medicament, drug products and kits disclosed herein, wherein the first composition is prepared by treating with ionizing radiation, such as gamma radiation, to deliver an absorbed radiation dose between about 1 kGy and about 50 kGy prior to being packaged. In another aspect is provided any of the methods, compositions for use as a medicament, drug products and kits disclosed herein, wherein the first composition is prepared by treating with ionizing radiation, such as gamma radiation, to deliver an absorbed radiation dose between about 1 kGy and about 45 kGy, or between about 1 kGy and about 40 kGy, between about 1 kGy and about 35 kGy, between about 1 kGy and about 30 kGy, or between about 1 kGy and about 25 kGy or between about 5 kGy and about 50 kGy, or between about 5 kGy and about 45 kGy, or between about 5 kGy and about 40 kGy, or between about 5 kGy and about 35 kGy, or between about 5 kGy and about 30 kGy, or between about 5 kGy and about 25 kGy, or between about 10 kGy and about 50 kGy, or between about 10 kGy and about 45 kGy, or between about 10 kGy and about 40 kGy, or between about 10 kGy and about 35 kGy, or between about 10 kGy and about 30 kGy, or between about 10 kGy and about 25 kGy, or between about 15 kGy and about 50 kGy, or between about 15 kGy and about 45 kGy, or between about 15 kGy and about 40 kGy, or between about 15 kGy and about 35 kGy, or between about 15 kGy and about 30 kGy, or between about 15 kGy and about 25 kGy. In another aspect is provided any of the methods, compositions for use as a medicament, drug products and kits disclosed herein, wherein the first composition is prepared by treating with ionizing radiation, such as gamma radiation, to deliver an absorbed radiation dose of about 1 kGy, or about 5 kGy, or about 10 kGy, 11 kGy, or about 12 kGy, or about 13 kGy, or about 14 kGy, or about 15 kGy, or about 16 kGy, or about 17 kGy, or about 18 kGy, or about 19 kGy, or about 20 kGy, or about 21 kGy, or about 22 kGy, or about 23 kGy, or about 24 kGy, or about 25 kGy, or about 26 kGy, or about 27 kGy, or about 28 kGy, or about 29 kGy, or about 30 kGy, or about 31 kGy, or about 32 kGy, or about 33 kGy, or about 34 kGy, or about 35 kGy, or about 36 kGy, or about 37 kGy, or about 38 kGy, or about 39 kGy, or about 40 kGy, or about 41 kGy, or about 42 kGy, or about 43 kGy, or about 44 kGy, or about 45 kGy, or about 46 kGy, or about 47 kGy, or about 48 kGy, or about 49 kGy, or about 50 kGy.

In another aspect is provided any of the methods, compositions for use as a medicament, drug products and kits disclosed herein, wherein the first composition is applied to the skin of the subject in the form of a paste. In another aspect is provided any of the methods, compositions for use as a medicament, drug products and kits disclosed herein, wherein the paste further comprises water or saline. In another aspect is provided any of the methods, compositions for use as a medicament, drug products and kits disclosed herein, wherein the paste is prepared by mixing a composition comprising Spongilla and an aqueous solution comprising hydrogen peroxide. In another aspect is provided any of the methods, compositions for use as a medicament, drug products and kits disclosed herein, wherein the hydrogen peroxide is at a concentration of from about 0.1% w/w to about 50% w/w, or from about 0.1% w/w to about 45% w/w, or from about 0.1% w/w to about 40% w/w, or from about 0.1% w/w to about 35% w/w, or from about 0.1% w/w to about 30% w/w, or from about 0.1% w/w to about 25% w/w, or from about 0.1% w/w to about 20% w/w, or from about 0.1% w/w to about 15% w/w, or from about 0.1% w/w to about 10% w/w, or from about 0.1% w/w to about 9% w/w, or from about 0.1% w/w to about 8% w/w, or from about 0.1% w/w to about 8% w/w, or from about 0.1% w/w to about 7% w/w, or from about 0.1% w/w to about 6% w/w, or from about 0.1% w/w to about 5% w/w, or from about 0.1% w/w to about 4% w/w, or from about 0.1% w/w to about 3% w/w, or from about 0.1% w/w to about 2% w/w, or from about 0.1% w/w to about 1% w/w, or from about 0.5% w/w to about 45% w/w, or from about 1% w/w to about 45% w/w, or from about 1% w/w to about 40% w/w, or from about 1% w/w to about 35% w/w, or from about 1% w/w to about 30% w/w, or from about 1% w/w to about 25% w/w, or from about 1% w/w to about 20% w/w, or from about 1% w/w to about 15% w/w, or from about 1% w/w to about 10% w/w, or from about 1% w/w to about 9% w/w, or from about 1% w/w to about 8% w/w, or from about 1% w/w to about 8% w/w, or from about 1% w/w to about 7% w/w, or from about 1% w/w to about 6% w/w, or from about 1% w/w to about 5% w/w, or from about 1% w/w to about 4% w/w, or from about 1% w/w to about 3% w/w, or from about 1% w/w to about 2% w/w, or from about 2% w/w to about 45% w/w, or from about 2% w/w to about 40% w/w, or from about 2% w/w to about 35% w/w, or from about 2% w/w to about 30% w/w, or from about 2% w/w to about 25% w/w, or from about 2% w/w to about 20% w/w, or from about 2% w/w to about 15% w/w, or from about 2% w/w to about 10% w/w, or from about 2% w/w to about 9% w/w, or from about 2% w/w to about 8% w/w, or from about 1% w/w to about 7% w/w, or from about 2% w/w to about 6% w/w, or from about 2% w/w to about 5% w/w, or from about 2% w/w to about 4% w/w, or from about 2% w/w to about 3% w/w. In another aspect is provided any of the methods, compositions for use as a medicament, drug products and kits disclosed herein, wherein the hydrogen peroxide is at a concentration of about 0.1% w/w, or about 0.5% w/w, or about 1% w/w, or about 2% w/w, or about 3% w/w, or about 4% w/w, or about 5% w/w, or about 6% w/w, or about 7% w/w, or about 8% w/w, or about 9% w/w, or about 10% w/w, or about 15% w/w, or about 20% w/w, or about 25% w/w, or about 30% w/w, or about 35% w/w, or about 40% w/w, or about 45% w/w, or about 50% w/w. In another aspect is provided any of the methods, compositions for use as a medicament, drug products and kits disclosed herein, wherein the hydrogen peroxide is at a concentration of about 3% w/w. Aqueous hydrogen peroxide solutions that may be useful in treating skin conditions in a subject as disclosed herein are commercially available or may be prepared by methods known to those of ordinary skill in the art.

In another aspect is provided any of the methods, compositions for use as a medicament, drug products and kits disclosed herein, wherein the first composition and/or the second composition may be used in combination with a gel or cream, which gel or cream may or may not further comprise hydrogen peroxide. In another aspect is provided any of the methods, compositions for use as a medicament, drug products and kits disclosed herein, wherein the first composition and/or the second composition may be used in combination with a gel or cream, which gel or cream does not further comprise hydrogen peroxide. In another aspect is provided any of the methods, compositions for use as a medicament, drug products and kits disclosed herein, wherein the first composition and/or the second composition may be used in combination with a gel or cream, which gel or cream further comprises hydrogen peroxide. Such gels or creams are generally commercially available any may contain from about 0.5% w/w to about 50% w/w hydrogen peroxide. For example, a gel containing about 1% w/w, or about 2% w/w, or about 3% w/w, or about 4% w/w, or about 6% w/w, or about 7% w/w, or about 8% w/w, or about 9% w/w, or about 10% w/w, or about 15% w/w, or about 20% w/w, or about 25% w/w, or about 30% w/w, or about 40% w/w, or about 45% w/w, or about 50% w/w hydrogen peroxide may be used in any of the methods disclosed herein in combination with the first composition and the second composition.

In another aspect is provided any of the methods, compositions for use as a medicament, drug products and kits disclosed herein, wherein the method further comprises applying a third composition to the skin of the subject. In another aspect is provided any of the methods, compositions for use as a medicament, drug products and kits disclosed herein, wherein the third composition comprises hydrogen peroxide. In another aspect is provided any of the methods, compositions for use as a medicament, drug products and kits disclosed herein, wherein the hydrogen peroxide is at a w/w concentration of about 3%. In another aspect is provided any of the methods, compositions for use as a medicament, drug products and kits disclosed herein, wherein the hydrogen peroxide is at a w/w concentration of about 0.1% w/w to about 50% w/w, or from about 0.1% w/w to about 45% w/w, or from about 0.1% w/w to about 40% w/w, or from about 0.1% w/w to about 35% w/w, or from about 0.1% w/w to about 30% w/w, or from about 0.1% w/w to about 25% w/w, or from about 0.1% w/w to about 20% w/w, or from about 0.1% w/w to about 15% w/w, or from about 0.1% w/w to about 10% w/w, or from about 0.1% w/w to about 9% w/w, or from about 0.1% w/w to about 8% w/w, or from about 0.1% w/w to about 8% w/w, or from about 0.1% w/w to about 7% w/w, or from about 0.1% w/w to about 6% w/w, or from about 0.1% w/w to about 5% w/w, or from about 0.1% w/w to about 4% w/w, or from about 0.1% w/w to about 3% w/w, or from about 0.1% w/w to about 2% w/w, or from about 0.1% w/w to about 1% w/w, or from about 0.5% w/w to about 45% w/w, or from about 1% w/w to about 45% w/w, or from about 1% w/w to about 40% w/w, or from about 1% w/w to about 35% w/w, or from about 1% w/w to about 30% w/w, or from about 1% w/w to about 25% w/w, or from about 1% w/w to about 20% w/w, or from about 1% w/w to about 15% w/w, or from about 1% w/w to about 10% w/w, or from about 1% w/w to about 9% w/w, or from about 1% w/w to about 8% w/w, or from about 1% w/w to about 8% w/w, or from about 1% w/w to about 7% w/w, or from about 1% w/w to about 6% w/w, or from about 1% w/w to about 5% w/w, or from about 1% w/w to about 4% w/w, or from about 1% w/w to about 3% w/w, or from about 1% w/w to about 2% w/w, or from about 2% w/w to about 45% w/w, or from about 2% w/w to about 40% w/w, or from about 2% w/w to about 35% w/w, or from about 2% w/w to about 30% w/w, or from about 2% w/w to about 25% w/w, or from about 2% w/w to about 20% w/w, or from about 2% w/w to about 15% w/w, or from about 2% w/w to about 10% w/w, or from about 2% w/w to about 9% w/w, or from about 2% w/w to about 8% w/w, or from about 1% w/w to about 7% w/w, or from about 2% w/w to about 6% w/w, or from about 2% w/w to about 5% w/w, or from about 2% w/w to about 4% w/w, or from about 2% w/w to about 3% w/w. In another aspect is provided any of the methods, compositions for use as a medicament, drug products and kits disclosed herein, wherein the hydrogen peroxide is at a concentration of about 0.1% w/w, or about 0.5% w/w, or about 1% w/w, or about 2% w/w, or about 3% w/w, or about 4% w/w, or about 5% w/w, or about 6% w/w, or about 7% w/w, or about 8% w/w, or about 9% w/w, or about 10% w/w, or about 15% w/w, or about 20% w/w, or about 25% w/w, or about 30% w/w, or about 35% w/w, or about 40% w/w, or about 45% w/w, or about 50% w/w. In another aspect is provided any of the methods, compositions for use as a medicament, drug products and kits disclosed herein, wherein the hydrogen peroxide is at a concentration of about 3% w/w. Aqueous hydrogen peroxide solutions that may be useful in treating skin conditions in a subject as disclosed herein are commercially available or may be prepared by methods known to those of ordinary skill in the art.

In another aspect is provided any of the methods, compositions for use as a medicament, drug products and kits disclosed herein, wherein the Spongilla comprises Spongilla lacustris.

In another aspect is provided any of the methods, compositions for use as a medicament, drug products and kits disclosed herein, wherein the amount of the first composition comprising Spongilla applied to the skin of the subject is from about 0.5 grams to about 50 grams. In one aspect is provided any of the methods disclosed herein, wherein the amount of the first composition is measured as a dry weight. In another aspect is provided any of the methods, compositions for use as a medicament, drug products and kits disclosed herein, wherein the amount of the first composition comprising Spongilla applied to the skin of the subject is from about 0.5 grams to about 40 grams, or from about 0.5 grams to about 35 grams, or from about 0.5 grams to about 30 grams, or from about 0.5 grams to about 25 grams, or from about 0.5 grams to about 20 grams, or from about 0.5 grams to about 15 grams, or from about 0.5 grams to about 10 grams, or from about 0.75 grams to about 20 grams, or from about 0.75 grams to about 15 grams, or from about 0.75 grams to about 10 grams, or from about 1 gram to about 20 grams, or from about 1 gram to about 15 grams, or from about 1 gram to about 10 grams, or from about 1 gram to about 9 grams, or from about 1 gram to about 8 grams, or from about 1 gram to about 7 grams, or from about 1 gram to about 6 grams, or from about 1 gram to about 5 grams, or from about 1 gram to about 4 grams, or from about 1 gram to about 3 grams, or from about 1 gram to 2 grams. In another aspect is provided any of the methods, compositions for use as a medicament, drug products and kits disclosed herein wherein the amount of Spongilla applied to the skin, such as those disclosed above, are each measured as a dry weight.

In another aspect is provided any of the methods, compositions for use as a medicament, drug products and kits disclosed herein, wherein the amount of the first composition comprising Spongilla applied to the skin of the subject is about 0.5 grams, or about 0.75 grams, or about 1 gram, or about 1.25 grams, or about 1.5 grams, or about 1.75 grams, or about 2 grams, or about 2.25 grams, or about 2.5 grams, or about 2.75 grams, or about 3 grams, or about 3.25 grams, or about 3.5 grams, or about 3.75 grams, or about 4 grams, or about 4.25 grams, or about 4.5 grams, or about 4.75 grams, or about 5 grams, or about 5.25 grams, or about 5.5 grams, or about 5.75 grams, or about 6 grams, or about 6.25 grams, or about 6.5 grams, or about 7 grams, or about 7.25 grams, or about 7.5 grams, or about 7.75 grams, or about 8 grams, or about 8.25 grams, or about 8.5 grams, or about 8.75 grams, or about 9 grams, or about 9.25 grams, or about 9.5 grams, or about 9.75 grams, or about 10 grams, or about 11 grams, or about 12 grams, or about 13 grams, or about 14 grams, or about 15 grams, or about 16 grams, or about 17 grams, or about 18 grams, or about 19 grams, or about 20 grams, or about 25 grams, or about 35 grams, or about 40 grams, or about 45 grams, or about 50 grams, or about 75 grams, or about 100 grams, or about 250 grams, or about 500 grams, or about 750 grams, or about 1000 grams, in each case measured as a dry weight.

In another aspect is provided any of the methods, compositions for use as a medicament, drug products and kits disclosed herein, wherein the first composition is applied to the skin of the subject prior to the second composition being applied to the skin of the subject. In another aspect is provided any of the methods, compositions for use as a medicament, drug products and kits disclosed herein, wherein the first composition is applied to the skin of the subject and is permitted to dry on the skin of the subject prior to application of the second composition to the skin of the subject. In another aspect is provided any of the methods, compositions for use as a medicament, drug products and kits disclosed herein, wherein the first composition is applied to the skin of the subject in the form of an aqueous paste, wherein the aqueous component may be water or saline. In another aspect is provided any of the methods, compositions for use as a medicament, drug products and kits disclosed herein, wherein the aqueous paste further comprises hydrogen peroxide. In another aspect is provided any of the methods, compositions for use as a medicament, drug products and kits disclosed herein, wherein an aqueous solution of hydrogen peroxide is applied to the skin of the subject following application of the first composition to the skin of the subject and prior to the application of the second composition to the skin of the subject. In another aspect is provided any of the methods, compositions for use as a medicament, drug products and kits disclosed herein, wherein the first composition further comprises hydrogen peroxide. In another aspect is provided any of the methods, compositions for use as a medicament, drug products and kits disclosed herein, wherein the hydrogen peroxide is an aqueous solution. In another aspect is provided any of the methods, compositions for use as a medicament, drug products and kits disclosed herein, wherein the aqueous solution comprises hydrogen peroxide at a w/w concentration of about 3%. In another aspect is provided any of the methods, compositions for use as a medicament, drug products and kits disclosed herein, wherein the second composition is permitted to dry on the skin of the subject following application to the skin of the subject.

In another aspect is provided any of the methods, compositions for use as a medicament, drug products and kits disclosed herein, wherein the second composition is applied to the skin of the subject prior to the first composition being applied to the skin of the subject. In another aspect is provided any of the methods, compositions for use as a medicament, drug products and kits disclosed herein, wherein the second composition is permitted to dry on the skin of the subject prior to the first composition being applied to the skin of the subject. In another aspect is provided any of the methods, compositions for use as a medicament, drug products and kits disclosed herein, wherein the first composition is applied to the skin of the subject in the form of an aqueous paste, wherein the aqueous portion may be derived from water or saline. In another aspect is provided any of the methods, compositions for use as a medicament, drug products and kits disclosed herein, wherein the aqueous paste further comprises hydrogen peroxide. In another aspect is provided any of the methods, compositions for use as a medicament, drug products and kits disclosed herein, wherein an aqueous solution of hydrogen peroxide is applied to the skin of the subject following application of the first composition to the skin of the subject. In another aspect is provided any of the methods, compositions for use as a medicament, drug products and kits disclosed herein, wherein the hydrogen peroxide is an aqueous solution. In another aspect is provided any of the methods, compositions for use as a medicament, drug products and kits disclosed herein, wherein the aqueous solution comprises hydrogen peroxide at a w/w concentration of about 3%. In another aspect is provided any of the methods, compositions for use as a medicament, drug products and kits disclosed herein, wherein the first composition is permitted to dry on the skin of the subject.

In another aspect is provided any of the methods, compositions for use as a medicament, drug products and kits disclosed herein, wherein the first composition and the second composition are mixed together and the resulting mixture is applied to the skin of the subject. In another aspect is provided any of the methods, compositions for use as a medicament, drug products and kits disclosed herein, wherein the first composition is mixed with an aqueous solution of hydrogen peroxide prior to mixing with the second composition. In another aspect is provided any of the methods, compositions for use as a medicament, drug products and kits disclosed herein, wherein the mixture of the first composition and the second composition is further mixed with an aqueous solution of hydrogen peroxide prior to application to the skin of the subject. In another aspect is provided any of the methods, compositions for use as a medicament, drug products and kits disclosed herein, wherein the aqueous solution comprises hydrogen peroxide at a w/w concentration of about 3%. In another aspect is provided any of the methods, compositions for use as a medicament, drug products and kits disclosed herein, wherein the first composition and the second composition are in a single mixture and the mixture is applied to the skin of the subject.

In another aspect is provided any of the methods, compositions for use as a medicament, drug products and kits disclosed herein, wherein the subject applies the second composition comprising Spongilla to the skin no more than once every 4 weeks. In another aspect is provided any of the methods, compositions for use as a medicament, drug products and kits disclosed herein, wherein the first composition comprising Spongilla is applied to the skin of the subject at least once per week for at least one week. In another aspect is provided any of the methods, compositions for use as a medicament, drug products and kits disclosed herein, wherein the first composition comprising Spongilla is applied to the skin of the subject at least at least two times per week for at least one week, at least three times per week for at least one week, at least 4 times per week for at least one week, at least 5 times per week for at least one week, at least 6 times per week for at least one week, or at least 7 times per week for at least one week.

In another aspect is provided any of the methods, compositions for use as a medicament, drug products and kits disclosed herein, wherein the first composition comprising Spongilla is applied to the skin of the subject at least once per week for at least two weeks. In another aspect is provided any of the methods, compositions for use as a medicament, drug products and kits disclosed herein, wherein the first composition comprising Spongilla is applied to the skin of the subject at least at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks. In another aspect is provided any of the methods, compositions for use as a medicament, drug products and kits disclosed herein, wherein the first composition comprising Spongilla is applied to the skin of the subject once per week for 6 weeks.

In another aspect is provided any of the methods, compositions for use as a medicament, drug products and kits disclosed herein, wherein the first composition comprising Spongilla is applied to the skin of the subject once per week for 24 weeks, and the second composition comprising one or more drugs is applied to the skin of the subject only during the first week of treatment. In another aspect is provided any of the methods, compositions for use as a medicament, drug products and kits disclosed herein, wherein the first composition comprising Spongilla is applied to the skin of the subject once per week for 20 weeks, and the second composition comprising one or more drugs is applied to the skin of the subject only during the first week of treatment. In another aspect is provided any of the methods, compositions for use as a medicament, drug products and kits disclosed herein, wherein the first composition comprising Spongilla is applied to the skin of the subject once per week for 16 weeks, and the second composition comprising one or more drugs is applied to the skin of the subject only during the first week of treatment. In another aspect is provided any of the methods, compositions for use as a medicament, drug products and kits disclosed herein, wherein the first composition comprising Spongilla is applied to the skin of the subject once per week for 12 weeks, and the second composition comprising one or more drugs is applied to the skin of the subject only during the first week of treatment. In another aspect is provided any of the methods, compositions for use as a medicament, drug products and kits disclosed herein, wherein the first composition comprising Spongilla is applied to the skin of the subject once per week for 8 weeks, and the second composition comprising one or more drugs is applied to the skin of the subject only during the first week of treatment. In another aspect is provided any of the methods, compositions for use as a medicament, drug products and kits disclosed herein, wherein the first composition comprising Spongilla is applied to the skin of the subject once per week for 6 weeks, and the second composition comprising one or more drugs is applied to the skin of the subject only during the first week of treatment. In another aspect is provided any of the methods, compositions for use as a medicament, drug products and kits disclosed herein, wherein the first composition comprising Spongilla is applied to the skin of the subject once per week for 4 weeks, and the second composition comprising one or more drugs is applied to the skin of the subject only during the first week of treatment.

In another aspect is provided any of the methods, compositions for use as a medicament, drug products and kits disclosed herein, wherein the first composition comprising Spongilla and the second composition comprising one or more drugs are applied to the skin of the subject in combination once per week, once every two weeks, once every three weeks, once every four weeks, once every five weeks, once every six weeks, once every 7 weeks, once every 8 weeks, once every 9 weeks, once every 10 weeks, once every 11 weeks, or once every 12 weeks. In another aspect is provided any of the methods, compositions for use as a medicament, drug products and kits disclosed herein, wherein the first composition comprising Spongilla and the second composition comprising one or more drugs are applied to the skin of the subject in combination once per week. In another aspect is provided any of the methods, compositions for use as a medicament, drug products and kits disclosed herein, wherein the first composition comprising Spongilla and the second composition comprising one or more drugs are applied to the skin of the subject in combination once every two weeks. In another aspect is provided any of the methods, compositions for use as a medicament, drug products and kits disclosed herein, wherein the first composition comprising Spongilla and the second composition comprising one or more drugs are applied to the skin of the subject in combination once every three weeks. In another aspect is provided any of the methods, compositions for use as a medicament, drug products and kits disclosed herein, wherein the first composition comprising Spongilla and the second composition comprising one or more drugs are applied to the skin of the subject in combination once every four weeks. In another aspect is provided any of the methods, compositions for use as a medicament, drug products and kits disclosed herein, wherein the first composition comprising Spongilla and the second composition comprising one or more drugs are applied to the skin of the subject in combination once every five weeks. In another aspect is provided any of the methods, compositions for use as a medicament, drug products and kits disclosed herein, wherein the first composition comprising Spongilla and the second composition comprising one or more drugs are applied to the skin of the subject in combination once every six weeks. In another aspect is provided any of the methods, compositions for use as a medicament, drug products and kits disclosed herein, wherein the first composition comprising Spongilla and the second composition comprising one or more drugs are applied to the skin of the subject in combination once every 7 weeks. In another aspect is provided any of the methods, compositions for use as a medicament, drug products and kits disclosed herein, wherein the first composition comprising Spongilla and the second composition comprising one or more drugs are applied to the skin of the subject in combination once every 8 weeks. In another aspect is provided any of the methods, compositions for use as a medicament, drug products and kits disclosed herein, wherein the first composition comprising Spongilla and the second composition comprising one or more drugs are applied to the skin of the subject in combination once every 9 weeks. In another aspect is provided any of the methods, compositions for use as a medicament, drug products and kits disclosed herein, wherein the first composition comprising Spongilla and the second composition comprising one or more drugs are applied to the skin of the subject in combination once every 10 weeks. In another aspect is provided any of the methods, compositions for use as a medicament, drug products and kits disclosed herein, wherein the first composition comprising Spongilla and the second composition comprising one or more drugs are applied to the skin of the subject in combination once every 11 weeks. In another aspect is provided any of the methods, compositions for use as a medicament, drug products and kits disclosed herein, wherein the first composition comprising Spongilla and the second composition comprising one or more drugs are applied to the skin of the subject in combination once every 12 weeks.

In another aspect is provided any of the methods, compositions for use as a medicament, drug products and kits disclosed herein, wherein the skin of the subject is cleaned using a non-comedogenic cleanser, water, or a combination of a non-comedogenic cleanser and water following application of the first composition comprising Spongilla. In another aspect is provided any of the methods, compositions for use as a medicament, drug products and kits disclosed herein, wherein the skin of the subject is cleaned using a non-comedogenic cleanser, water, or a combination of a non-comedogenic cleanser and water following application of the second composition comprising one or more drugs to the skin of the subject. Non-comedogenic cleansers are those formulated not to cause blocked pores in the skin of subjects to which such cleansers are applied.

In another aspect is provided any of the methods, compositions for use as a medicament, drug products and kits disclosed herein, wherein the subject is a mammal, including but not limited to humans, dogs, cats, cattle, sheep, and goats. In some embodiments, the subject is a human. In some embodiments, the subject is a dog. In some embodiments, the subject is a cat. In some embodiments, the subject is a cow. In some embodiments, the subject is a cat. In some embodiments, the subject is a sheep. In some embodiments, the subject is a cat. In some embodiments, the subject is a goat.

Plaque psoriasis is the most common form of the disease and appears as raised, red patches covered with a silvery white buildup of dead skin cells or scale. These patches or plaques most often appear on the scalp, knees, elbows and lower back. They are often itchy and painful, and they can crack and bleed. Plaque psoriasis is most often found on the outside of knees and elbows, the scalp, the lower back, the face, the palms and soles of feet. When biopsied, psoriasis skin looks thicker and inflamed when compared with eczema.

Psoriatic arthritis is a form of arthritis that affects some people who have psoriasis and can present in subjects as red patches of skin topped with silvery scales. Most people develop psoriasis first and are later diagnosed with psoriatic arthritis, but the joint problems can sometimes begin before skin lesions appear. The most common symptoms in subjects are joint pain, stiffness and swelling and can affect a subject's fingertips and spine and can range from relatively mild to severe. In both psoriasis and psoriatic arthritis, disease flares may alternate with periods of remission.

Atopic dermatitis (eczema) is a condition that causes a subject's skin to become red, dry and itchy. Red to brownish-gray patches may appear on a subject's skin, especially on the hands, feet, ankles, wrists, neck, upper chest, eyelids, inside the bend of the elbows and knees, and in infants, the face and scalp. Small, raised bumps, which may leak fluid and crust over when scratched. The condition is usually chronic, tends to flare periodically, and may be accompanied by asthma or hay fever.

Acne vulgaris is a common chronic skin disease involving blockage and/or inflammation of pilosebaceous units (hair follicles and their accompanying sebaceous gland). Acne can present as noninflammatory lesions, inflammatory lesions, or a mixture of both, affecting mostly the face but also the back and chest. The efficacy of a treatment regimen in a subject having acne vulgaris can be measured by methods known to those of ordinary skill in the art, such as by measurement of lesion counts and the investigator global assessment on the face in a subject such as found below:

| Score | Grade | Description |
|---|---|---|
| 0 | None | No evidence of facial acne vulgaris |
| 1 | Minimal | Few non-inflammatory lesions |
| | | (comedones) are present; a few |
| | | inflammatory lesions (papules/pustules) |
| | | may be present; no nodulo-cystic |
| | | lesions are allowed |
| 2 | Mild | Several to many non-inflammatory |
| | | lesions (comedones) are present; |
| | | a few inflammatory lesions |
| | | (papules/pustules) are present; |
| | | no nodulo-cystic lesions are allowed |
| 3 | Moderate | Many non-inflammatory lesions (comedones) |
| | | and inflammatory lesions (papules/pustules) |
| | | are present; no nodulo-cystic lesions |
| | | are allowed |
| 4 | Severe | Significant degree of inflammatory disease; |
| | | papules/pustules are a predominate feature; |
| | | a few nodulo-cystic lesions may be |
| | | present; comedones may be present |

Rosacea is well recognized as a chronic cutaneous disorder primarily of the convexities of the central face (cheeks, chin, nose, and central forehead), often characterized by remissions and exacerbations. Based on present knowledge, it is considered a syndrome, or typology, encompassing various combinations of such cutaneous signs as flushing, erythema, telangiectasia, edema, papules, pustules, ocular lesions, and rhinophyma. In most cases, some rather than all of these stigmata appear in any given subject. Acne rosacea type-1, or erythematotelangiectatic rosacea, is mainly characterized by flushing and persistent central facial erythema. The appearance of telangiectases is common but not essential for a diagnosis of this subtype. Central facial edema, stinging and burning sensations, and roughness or scaling may also be reported. A history of flushing alone is common among subjects presenting with erythematotelangiectatic rosacea. The efficacy of a treatment regimen in a subject having acne rosacea type-lcan be measured by methods known to those of ordinary skill in the art, such as by use of the Clinician Erythema Assessment (CEA), a 5-point grading scale of facial erythema severity, and Subject Self-Assessment (SSA) shown below:

| Grade | Category | Description |
|---|---|---|
| 0 | None | Clear skin with no signs of erythema |
| 1 | Minimal | Almost clear of erythema, slight redness |
| 2 | Mild | Mild erythema, definite redness |
| 3 | Moderate | Moderate erythema, marked redness |
| 4 | Severe | Severe erythema, fiery redness |

| Grade | Category | Description |
|---|---|---|
| 0 | None | Clear of redness |
| 1 | Minimal | Almost clear of redness |
| 2 | Mild | Somewhat more redness than preferred |
| 3 | Moderate | More redness than preferred |
| 4 | Severe | Completely unacceptable redness |

Acne rosacea type 2 (papulopustular) is characterized by persistent central facial erythema with transient papules or pustules or both in a central facial distribution. However, papules and pustules also may occur periorificially (that is, they may occur in the perioral, perinasal, or periocular areas). The papulopustular subtype resembles acne vulgaris, except that comedones are absent. Rosacea and acne may occur concomitantly, and such subjects may have comedones as well as the papules and pustules of rosacea. Burning and stinging sensations may be reported by subjects with papulopustular rosacea. This subtype has often been seen after or in combination with subtype 1, including the presence of telangiectases. The telangiectases may be obscured by persistent erythema, papules, or pustules, and tend to become more visible after successful treatment of these masking components. The efficacy of a treatment regimen in a subject having acne rosacea type-2 can be measured by methods known to those of ordinary skill in the art, such as by total lesion counts in the area of the skin of the subject undergoing treatment and an investigator global assessment as shown below:

| Grade | Category | Description |
|---|---|---|
| 0 | Clear | No papules and/or pustules |
| 1 | Almost | Clear Rare papules and/or pustules |
| 2 | Mild | Few papules and/or pustules |
| 3 | Moderate | Pronounced number of papules |
| | | and/or pustules (but less than |
| | | numerous papules and/or pustules) |
| 4 | Severe | Numerous papules and/or pustules, |
| | | occasionally with confluent |
| | | areas of inflamed lesions |

Psoriasis is a skin condition that speeds up the life cycle of skin cells. It causes cells to build up rapidly on the surface of the skin. The extra skin cells form scales and red patches that are itchy and sometimes painful. The symptoms a subject having psoriasis may present include red patches of skin covered with thick, silvery scales, small scaling spots (commonly seen in children), dry, cracked skin that may bleed, itching, burning or soreness, thickened, pitted or ridged nails, and swollen and stiff joints. The efficacy of a treatment regimen in a subject having acne psoriasis can be measured by methods known to those of ordinary skill in the art, such as by use of the Psoriasis Area and Severity Index (PASI). Use of PASI involves dividing the body of the subject into four sections (head (H) (10% of a person's skin); arms (A) (20%); trunk (T) (30%); legs (L) (40%)). Each of these areas is scored by itself, and then the four scores are combined into the final PASI. For each section, the percent of area of skin involved, is estimated and then transformed into a grade from 0 to 6 as in the table below. Within each area, the severity is estimated by three clinical signs: erythema (redness), induration (thickness) and desquamation (scaling). Severity parameters are measured on a scale of 0 to 4, from none to maximum. The sum of all three severity parameters is then calculated for each section of skin, multiplied by the area score for that area and multiplied by weight of respective section (0.1 for head, 0.2 for arms, 0.3 for body and 0.4 for legs).

| Score | Description |
|---|---|
| 0 | 0% of involved area |
| 1 | <10% of involved area |
| 2 | 10-29% of involved area |
| 3 | 30-49% of involved area |
| 4 | 50-69% of involved area |
| 5 | 70-89% of involved area |
| 6 | 90-100% of involved area |

Hyperhidrosis is a condition characterized by abnormally increased sweating, generally in excess of that required for regulation of body temperature. Although primarily a physical burden, hyperhidrosis can deteriorate quality of life from a psychological, emotional, and social perspective. The efficacy of a treatment regimen in a subject having hyperhidrosis can be measured by methods known to those of ordinary skill in the art, such as by use of the hyperhidrosis disease severity scale (HDSS), which is a 4-point scale designed to assess the severity of primary axillary hyperhidrosis in everyday clinical practice or in clinical research. The HDSS can be administered by an interviewer or self-completed by the subject. The HDSS assesses subject severity based on the extent of excessive sweating-related impairment of daily activities. Subjects rate the severity as: 1=my underarm sweating is never noticeable and never interferes with my daily activities; 2=my underarm sweating is tolerable but sometimes interferes with my daily activities; 3=my underarm sweating is barely tolerable and frequently interferes with my daily activities; or 4=my underarm sweating is intolerable and always interferes with my daily activities. The efficacy of a treatment regimen in a subject having hyperhidrosis may also be measured use of gravimetric sweat production, which can be done on the palm, the axilla, feet and other parts of the body. Generally, this method is performed by drying the body surface on which sweat production is to be measured and applying a preweighed filter paper to the body surface of interest for a predetermined period of time, after which the paper is removed, weighed and the rate of sweat production is calculated in milligrams of sweat in the amount of time measured.

Alopecia areata is an autoimmune skin disease, causing hair loss on the scalp, face and sometimes on other areas of the body. In fact, it affects as many as 6.8 million people in the U.S. The efficacy of a treatment regimen in a subject having alopecia areata can be measured by methods known to those of ordinary skill in the art, such as by use fixed hair counts, and loose hair counts on a subject's pillow.

Androgenic alopecia is a genetically determined disorder characterized by the gradual conversion of terminal hairs into indeterminate, and finally into vellus, hairs. It is an extremely common disease that affects men and women. Subjects suffering from androgenic alopecia generally display symptoms such as a gradual onset of hair loss, increased hair shedding, transition in the involved areas from large, thick, pigmented terminal hairs to thinner, shorter, indeterminate hairs and finally to short, wispy, nonpigmented vellus hairs, the end result of which may be an area of total denudation; this area varies from subject to subject and is usually most marked at the vertex. The efficacy of a treatment regimen in a subject having androgenic alopecia can be measured by methods known to those of ordinary skill in the art, such as by use fixed hair counts, and loose hair counts on a subject's pillow.

Keloids are raised, reddish nodules that develop at the site of an injury. After a wound has occurred to the skin both skin cells and connective tissue cells (fibroblasts) begin multiplying to repair the damage. A scar is made up of ‘connective tissue’, gristle-like fibers deposited in the skin by the fibroblasts to hold the wound closed. With keloids, the fibroblasts continue to multiply even after the wound is filled in. Thus, keloids project above the surface of the skin and form large mounds of scar tissue. Keloids may form on any part of the body, although the upper chest, shoulders and upper back are especially prone to keloid formation. Symptoms include pigmentation of the skin, itchiness, redness, unusual sensations and pain. Darkly pigmented people seem to be more prone to forming keloids. Men and women are equally affected. Keloids are considered a benign tumor, but they are mainly a cosmetic nuisance and never become malignant. Operating on a keloid usually stimulates more scar tissue to form; so many subjects having keloids may be told that there are no available treatments. Hypertrophic scars appear like, and are more common than, keloids, although they do not generally grow as large as keloids, may fade with time, and occur in all racial groups. The efficacy of a treatment regimen in a subject having keloids and/or hypertrophic scars can be measured by methods known to those of ordinary skill in the art, such as by the use the Vancouver Scar Scale (VSS), Manchester Scar Scale (MSS), Subject and Observer Scar Assessment Scale (POSAS), Visual Analog Scale (VAS), and Stony Brook Scar Evaluation Scale (SBSES).

Hidradenitis suppurativa is a disease that usually begins as pimple-like bumps on the skin, which tend to develop in places that everyday pimples do not appear and is most common on the underarms and groin. If hidradenitis suppurativa worsens, the pimple-like bumps can grow deep into the skin and become painful and can rupture. As the deep bumps heal, scars can form, and some subjects develop tunnel-like tracts under their skin, forming scars, which can thicken. When thick scars form in the underarm, moving the arm can be difficult. Thick scars in the groin area can make walking difficult. The efficacy of a treatment regimen in a subject suffering from hidradenitis suppurativa can be measured by methods known to those of ordinary skill in the art, such as by the visual count of lesion counts in the affected areas of a subject's skin.

Raynaud's phenomenon is a type of vascular disease characterized by a pale to blue to red sequence of color changes of the digits, most commonly after exposure to cold. The cause of Raynaud's phenomenon is unknown, although abnormal nerve control of blood-vessel diameter and nerve sensitivity to cold are suspected of being involved. Symptoms of Raynaud's phenomenon depend on the severity, frequency, and duration of the blood-vessel spasm. The efficacy of a treatment regimen in a subject suffering from Raynaud's phenomenon can be measured by methods known to those of ordinary skill in the art, such as measurements of digital pulp temperature, photographic assessment of the affected areas, and a visual analogue scale for pain in the affected areas.

Post-herpetic neuralgia is generally considered a complication of shingles, which is caused by the chickenpox (herpes zoster) virus. Postherpetic neuralgia affects nerve fibers and skin, causing burning pain that lasts long after the rash and blisters of shingles disappear. The signs and symptoms of postherpetic neuralgia are generally limited to the area in a subject's skin where the shingles outbreak first occurred. Signs and symptoms of post-herpetic neuralgia may include pain that lasts 3 months or longer after the shingles rash has healed sensitivity to light touch, and itching and numbness in the affected area. The efficacy of a treatment regimen in a subject suffering from post-herpetic neuralgia can be measured by methods known to those of ordinary skill in the art, use of a visual analogue scale for pain in the affected areas.

Hailey-Hailey Disease (familial benign pemphigus) is a genetic disorder that causes blisters to form on the skin and is characterized by outbreaks of rashes and blisters in the skin, usually in the folds of the skins, but also often over large areas of the body. The painful blisters break and sometimes become infected and raw, with new blisters forming over raw skin in a sometimes seemingly unending cycle of outbreaks. The cause of the disease is a haploinsufficiency of the enzyme ATP2C1, which encodes the protein hSPCA1. A mutation on one copy of the gene causes only half of this necessary protein to be made and the cells of the skin do not adhere together properly due to malformation of intercellular desmosomes, causing acantholysis, blisters and rashes. The efficacy of a treatment regimen in a subject suffering from Hailey-Hailey Disease can be measured by methods known to those of ordinary skill in the art, such as counting the total lesion counts in a subject, wherein a reduction in the total lesion count indicates the treatment regimen is having a positive effect.

Linear IgA bullous dermatosis (LABD) is a rare subepidermal blistering disease due to an autoimmune reaction against basement membrane proteins such as the lamina lucida and sublamina densa. The basement membrane anchors the epidermis to the dermis and helps to stabilize the skin. When IgA antibodies target such proteins, the basement membrane destabilizes resulting in tense blister formation. In the majority of LABD cases, the cause is unknown or idiopathic. Furthermore, more than half of all childhood cases tend to remit over a mean course of two to four years. Adults may have a more protracted course and LABD has been shown to occur in those with internal malignancy, infection, and other autoimmune diseases like rheumatoid arthritis or dermatomyositis. Other cases of LABD are drug-induced often due to vancomycin and subjects can break out as early after the first dose of vancomycin in some cases. The efficacy of a treatment regimen in a subject suffering from LABD can be measured by methods known to those of ordinary skill in the art, such as counting the total lesion counts in a subject, wherein a reduction in the total lesion count indicates the treatment regimen is having a positive effect.

Epidermolysis bullosa simplex (EBS) is a chronic vesicular disorder with characteristic manifestations, from birth to infancy, of intraepidermal vesicle and milia formation on the hand, elbow, or knee due to minimal trauma. It is a genetic disorder that is caused by a dominant-negative mutation in either the keratin 5 (KRT5) or the keratin 14 (KRT14) gene. EBS is sub-categorized by its clinical manifestation into the systemic (Koebner), localized (Weber-Cockayne), and herpetiform (Dowling-Meara)1 types. The localized type of EBS is the mildest form of the subtypes that involves easy development of vesicles on the palms and soles from minimal mechanical trauma. According to molecular genetic studies of EBS, there are mutations in KRT5 and KRT14, which contribute to skeletons on hemidesmosome in keratinocytes located in the basal layer near the dermo-epidermal junction. Mutations in each subtype of EBS vary in location and severity2,3. The efficacy of a treatment regimen in a subject suffering from EBS can be measured by methods known to those of ordinary skill in the art, such as counting the total lesion counts in a subject, wherein a reduction in the total lesion count indicates the treatment regimen is having a positive effect.

Darier Disease is a skin condition characterized by wart-like blemishes on the body. The blemishes are usually yellowish in color, hard to the touch, mildly greasy, and can emit a strong odor. The most common sites for blemishes are the scalp, forehead, upper arms, chest, back, knees, elbows, and behind the ear. The mucous membranes can also be affected, with blemishes on the roof of the mouth (palate), tongue, inside of the cheek, gums, and throat. Other features of Darier disease include nail abnormalities, such as red and white streaks in the nails with an irregular texture, and small pits in the palms of the hands and soles of the feet. The wart-like blemishes characteristic of Darier disease usually appear in late childhood to early adulthood. The severity of the disease varies over time; affected people experience flare-ups alternating with periods when they have fewer blemishes. The appearance of the blemishes is influenced by environmental factors. Most people with Darier disease will develop more blemishes during the summertime when they are exposed to heat and humidity. UV light; minor injury or friction, such as rubbing or scratching; and ingestion of certain medications can also cause an increase in blemishes. The efficacy of a treatment regimen in a subject suffering from Darier Disease can be measured by methods known to those of ordinary skill in the art, such as counting the total lesion counts and measuring the size of the lesions in a subject, wherein a reduction in the total lesion count indicates the treatment regimen is having a positive effect.

Pachyonchia Congenita is a condition that primarily affects the nails and skin. The signs and symptoms of this condition in a subject usually become apparent within the first few months of a subject's life. Almost everyone with pachyonychia congenita has hypertrophic nail dystrophy, which causes the fingernails and toenails to become thick and abnormally shaped. Many affected children also develop very painful blisters and calluses on the soles of the feet and, less commonly, on the palms of the hands. This condition is known as palmoplantar keratoderma. Severe blisters and calluses on the feet can make it painful or impossible to walk. Pachyonychia congenita can have several additional features, which vary among affected individuals. These features include thick, white patches on the tongue and inside of the cheeks (oral leukokeratosis); bumps called follicular keratoses that develop around hair follicles on the elbows, knees, and waistline; cysts in the armpits, groin, back, or scalp; and excessive sweating on the palms and soles (palmoplantar hyperhidrosis). Some affected individuals also develop widespread cysts called steatocystomas, which are filled with an oily substance called sebum that normally lubricates the skin and hair. Some babies with pachyonychia congenita have prenatal or natal teeth, which are teeth that are present at birth or in early infancy. Rarely, pachyonychia congenita can affect the voice box (larynx), potentially leading to hoarseness or breathing problems. The efficacy of a treatment regimen in a subject suffering from pachyonchia congenita can be measured by methods known to those of ordinary skill in the art, such as counting the total number of blisters and measuring the size of the blisters in the affected area on a subject.

Aquagenic keratoderma (AK) is a skin disorder also known as acquired aquagenic palmoplantar keratoderma, transient reactive papulotranslucent acrokeratoderma, aquagenic wrinkling of the palms or aquagenic syringeal acrokeratoderma. The main characteristic of the disorder is skin wrinkling with edema of palms/soles, whitish papules, pruritus, burning, and pain after contact with water. Prolongation of water exposure and temperature of the water affect the rate and intensity of lesion development; however, the pathogenesis of AK is poorly understood. The efficacy of a treatment regimen in a subject suffering from AK can be measured by methods known to those of ordinary skill in the art, such as counting the total number of lesions in a subject, the visual analogue pain score, and the visual analogue pruritis score.

Notalgia paresthetic is a sensory neuropathic syndrome of the midback skin, classically described as the unilateral infrascapular area. It is primarily a localized pruritus and dysesthesia syndrome, and it may present with episodic itching or pain on a small patch of the mid back, usually an area of skin just past easy reach. The correlation of notalgia paraesthetica localization with corresponding degenerative changes in the spine suggest that spinal nerve impingement may be a contributing cause, but subjects may have other conditions that predispose them to peripheral neuropathies, such as nerve damage. The efficacy of a treatment regimen in a subject suffering from notalgia paraesthetica can be measured by methods known to those of ordinary skill in the art, such as counting the total number of lesions in a subject, the visual analogue pain score, and the visual analogue pruritis score.

Pompholyx (dyshidrotic eczema) is a skin condition in which very small, fluid-filled blisters appear on the palms of a subject's hands, sides of the fingers, and soles of the feet. The blisters that occur in dyshidrosis may cause intense itching and, once dried, may cause a subject's skin to appear scaly. The blisters typically recur, sometimes before a subject's skin heals completely from the previous blisters. The efficacy of a treatment regimen in a subject suffering from dyshidrotic eczema can be measured by methods known to those of ordinary skill in the art, such as observing the signs and symptoms of eczema, the visual analogue pain score, and the visual analogue pruritis score.

Chromhidrosis is a condition characterized by the secretion of colored sweat and is caused by the deposition of lipofuscin in the sweat glands. It normally affects the apocrine glands, mainly on the face and underarms. The efficacy of a treatment regimen in a subject suffering from chromhidrosis can be measured by methods known to those of ordinary skill in the art, such as observing the signs of sweat and the odor of sweat in an affected subject.

Bromhidrosis, also known as osmidrosis, is a condition of abnormal or offensive body odor, largely determined by apocrine gland secretion, although other sources may play a role. Sudoriferous (sweat) glands are divided into two types: apocrine and eccrine and there is some crossover in some subjects. The efficacy of a treatment regimen in a subject suffering from bromhidrosis can be measured by methods known to those of ordinary skill in the art, such as observing the odor of sweat in an affected subject.

Eccrine nevus is a disease, which may be present at birth or at an early age. It is more often associated with localized hyperhidrosis, while cases not associated have also been reported. It is usually characterized histologically by the increase in number or size of structurally normal eccrine glands. The efficacy of a treatment regimen in a subject suffering from eccrine nevus can be measured by methods known to those of ordinary skill in the art, use of the Hyperhidrosis Disease Severity Scale (HDSS), and measuring the number sweat episodes per month in an affected subject.

Facial rhytides is a condition in subjects that is associated with moderate to severe glabellar lines associated with corrugator and/or procerus muscle activity, moderate to severe lateral canthal lines associated with orbicularis oculi activity, and/or moderate to severe forehead lines associated with frontalis muscle activity. The efficacy of a treatment regimen in a subject having facial rhytides can be measured by methods known to those of ordinary skill in the art, including a 4-point Facial Wrinkle Scale (FWS; 0=none, 1=mild, 2=moderate, 3=severe).

Atrophic acne scarring can occur in subjects suffering from acne. The efficacy of a treatment regimen in a subject suffering from atrophic acne scarring can be measured by methods known to those of ordinary skill in the art, including the Self-assessment of Clinical Acne-Related Scars (SCARS) and the Facial Acne Scar Quality of Life (FASQoL) tools.

Melasma is a skin condition that causes brown to gray-brown patches, usually on the face, including the cheeks, bridge of the nose, forehead, chin, and above the upper lip. It also can appear on other parts of the body that are exposed to sun, such as the forearms and neck.

Rheumatoid arthritis is a chronic inflammatory disorder that can affect a subject's joints, skin, eyes, lungs, heart and blood vessels. It is considered an autoimmune disorder that can affect the lining of a subject's joints, causing a painful swelling that can eventually result in bone erosion and joint deformity.

Lichen planus is an inflammatory condition that can affect a subject's skin, hair, nails and mucous membranes. On the skin, lichen planus usually appears as purplish, often itchy, flat-topped bumps, developing over several weeks. In the mouth, vagina and other areas covered by a mucous membrane, lichen planus forms lacy white patches, sometimes with painful sores. Lichen planus often occurs when a subject's immune system mistakenly attacks cells of the skin or mucous membranes and may be triggered by, hepatitis C infection, receiving a flu vaccine, exposure to certain pigments, chemicals and metals, exposure to nonsteroidal anti-inflammatory drugs (NSAIDs), such as ibuprofen and naproxen, and exposure to certain medications for heart disease, high blood pressure or arthritis.

Pityriasis rubra pilaris (PRP) refers to a group of skin conditions that cause constant inflammation and scaling of the skin. Subjects having PRP have reddish, scaly patches that may occur everywhere on the body, or only on certain areas, and some develop thickened skin on the underside of the hands and feet (palmoplantar keratoderma), various nail abnormalities, and/or thinning of the hair. There are several types of PRP classified by age when symptoms begin, body areas involved, and whether other conditions are present. The condition often occurs in adults (adult onset PRP) as well as children (juvenile onset PRP).

Ichthyosis is a family of rare genetic skin disorders characterized by dry, thickened, scaly skin characterized by rough, scaly skin. There are more than 20 types of ichthyosis which range in severity of symptoms, outward appearance, underlying genetic cause and mode of inheritance (e.g., whether the abnormal gene inherited is dominant, recessive, autosomal or X-linked). The severity of symptoms can vary, from the mildest, most common, types such as ichthyosis vulgaris, which may be mistaken for normal dry skin, up to life-threatening conditions such as harlequin-type ichthyosis. Ichthyosis vulgaris accounts for more than 95% of cases. In most cases, ichthyosis is not inherited and the cause is not known. In some people, particularly some with type V (“atypical juvenile type”), ichthyosis has autosomal dominant inheritance and may be caused by mutations in the CARD14 gene.

Palmoplantar pustulosis is a skin condition that occurs mostly in subjects who past or current smokers in which a subject's skin develops tiny fluid filled blisters on one or both hands and/or feet that fill with a small amount of pus, turn brown, then scaly and are associated with thickened, scaly, red skin that easily develops painful cracks (fissures). The condition varies in severity and may persist for many years and it is not known what triggers flare-ups.

In another aspect is provided a kit, comprising a first composition and a second composition, wherein (a) the first composition comprises a Spongilla; and (b) the second composition comprises one or more drugs. In another aspect is provided any of the kits described herein, further comprising instructions for use in treating the first and the second composition in the treatment in a subject having a skin disease condition. In another aspect is provided any of the kits described herein, wherein the first composition comprises Spongilla in the form of a powder. In another aspect is provided any of the kits described herein, wherein the Spongilla is in the form of a powder comprising particles that are substantially uniform in size.

In another aspect is provided any of the methods, compositions for use as a medicament, drug products and kits disclosed herein, wherein not less than 50% of the particles comprising the Spongilla powder pass through a US 70-mesh screen.

In another aspect is provided any of the methods, compositions for use as a medicament, drug products and kits disclosed herein, wherein not less than about 50% of the particles comprising the Spongilla powder pass through a US 70-mesh screen. In another embodiment are methods, wherein not less than about 60%, or about 70%, or about 75%, or about 80%, or about 85%, or about 90%, or about 95%, or about 96%, or about 97%, or about 98%, or about 99% of the particles comprising the Spongilla powder pass through a US 70-mesh screen. In another aspect is provided any of the kits disclosed herein, wherein not less than about 95%, or about 96%, or about 97%, or about 98%, or about 99% of the particles comprising the Spongilla powder pass through a US 70-mesh screen. In another aspect is provided any of the kits disclosed herein, wherein not less than about 95% of the particles comprising the Spongilla powder pass through a US 70-mesh screen. In another aspect is provided any of the kits disclosed herein, wherein not less than about 96% of the particles comprising the Spongilla powder pass through a US 70-mesh screen. In another aspect is provided any of the kits disclosed herein, wherein not less than about 97% of the particles comprising the Spongilla powder pass through a US 70-mesh screen. In another aspect is provided any of the kits disclosed herein, wherein not less than about 98% of the particles comprising the Spongilla powder pass through a US 70-mesh screen. In another aspect is provided any of the kits disclosed herein, wherein not less than about 99% of the particles comprising the Spongilla powder pass through a US 70-mesh screen.

In another aspect is provided any of the methods, compositions for use as a medicament, drug products and kits disclosed herein, wherein the particles comprising the Spongilla powder have an average length of from about 50 μm to about 500 μm. In another aspect is provided any of the kits disclosed herein, wherein the particles comprising the Spongilla powder have an average length of from about 50 μm to about 400 μm, or from about 50 μm to about 350 μm, or from about 50 μm to about 300 μm, or from about 50 μm to about 250 μm, or from about 50 μm to about 200 μm, or from about 75 μm to about 500 μm, or from about 75 μm to about 450 μm, or from about 80 μm to about 450 μm, or from about 80 μm to about 400 μm, or from about 85 μm to about 450 μm, or from about 85 μm to about 400 μm, or from about 90 μm to about 450 μm, or from about 90 μm to about 400 μm, or from about 90 μm to about 350 μm, or from about 100 μm to about 450 μm, or from about 100 μm to about 400 μm, or from about 100 μm to about 350 μm, or from about 100 μm to about 300 μm, or from about 100 μm to about 250 μm, or from about 100 μm to about 200 μm, or from about 150 μm to about 500 μm, or from about 100 μm to about 450 μm, or from about 150 μm to about 400 μm, or from about 150 μm to about 350 μm, or from about 150 μm to about 350 μm, or from about 150 μm to about 300 μm, or from about 150 μm to about 250 μm, or from about 150 μm to about 200 μm, or from about 175 μm to about 450 μm, or from about 175 μm to about 400 μm, or from about 175 μm to about 350 μm, or from about 175 μm to about 300 μm, or from about 175 μm to about 250 μm, or from about 175 μm to about 200 μm. In another aspect is provided any of the methods, compositions for use as a medicament, drug products and kits disclosed herein, wherein the particles comprising the Spongilla powder have an average length of about 50 μm, or about 75 μm, or about 80 μm, or about 85 μm, or about 90 μm, or about 100 μm, or about 125 μm, or about 150 μm, or about 175 μm, or about 200 μm, or about 225 μm, or about 250 μm, or about 300 μm, or about 350 μm, or about 400 μm, or about 450 μm, or about 500 μm In another aspect is provided any of the methods, compositions for use as a medicament, drug products and kits disclosed herein, wherein the particles comprising the Spongilla powder have an average length of about 200 μm.

In another aspect is provided any of the methods, compositions for use as a medicament, drug products and kits disclosed herein, wherein the particles comprising the Spongilla powder have an average diameter of from about 5 μm to about 50 μm In another aspect is provided any of the methods, compositions for use as a medicament, drug products and kits disclosed herein, wherein the particles comprising the Spongilla powder have an average diameter of from about 5 μm to about 45 μm, or from about 5 μm to about 40 μm, from about 5 μm to about 35 μm, from about 5 μm to about 30 μm, from about 5 μm to about 25 μm, from about 5 μm to about 20 μm, from about 10 μm to about 45 μm, from about 10 μm to about 40 μm, from about 10 μm to about 35 μm, from about 10 μm to about 30 μm, from about 10 μm to about 25 μm, from about 10 μm to about 20 μm. In another aspect is provided any of the methods, compositions for use as a medicament, drug products and kits disclosed herein, wherein the particles comprising the Spongilla powder have an average diameter of about 5 μm, or about 10 μm, or about 15 μm, or about 20 μm, or about 25 μm, or about 30 μm, or about 35 μm, or about 40 μm, or about 45 μm, or about 50 μm.

The compositions disclosed herein, such as the first composition comprising Spongilla, may further comprise one or more conventional pharmaceutical carriers or excipients. Suitable pharmaceutical carriers and excipients include inert diluents, binders (such as starches), fillers (such as colloidal silicon dioxide, sugars, including lactose, sucrose, mannitol, or sorbitol; and cellulose preparations, for example, maize starch, wheat starch, rice starch, potato starch, gelatin, gum, methyl cellulose, hydroxypropylmethyl cellulose, sodium carboxymethylcellulose, or polyvinylpyrrolidone (PVP)), bulking agents, lubricants (such as magnesium stearate, sodium lauryl sulfate and talc), coloring matters or dyes and, if desired, emulsifying agents or suspending agents, together with diluents such as water, saline, ethanol, propylene glycol, glycerin, or combinations thereof.

The pharmaceutical compositions disclosed herein may be in unit dosage forms suitable for single administration of precise dosages. In another aspect is provided any of the methods or kits disclosed herein, wherein the unit dosage forms of the first compositions and/or the second composition are suitable for two administrations, three administrations, four administrations, five administrations, six administrations, seven administrations, eight administrations, 9 administrations, 10 administrations, 11 administrations, 12 administrations, 13 administrations, 14 administrations, 15 administrations, 16 administrations, 17 administrations, 18 administrations, 19 administrations, 20 administrations, 21 administrations, 22 administrations, 23 administrations, 24 administrations, 25 administrations, 26 administrations, 27 administrations, 28 administrations, 29 administrations, 30 administrations, administrations for two months, administrations for three months, administrations for four months, administrations for five months, administrations for six months, administrations for seven months, administrations for eight months, administrations for nine months, administrations for ten months, administrations for eleven months, or administrations for 12 months.

It will be appreciated that the actual dosages of the compositions disclosed herein, may vary according to the composition being used, the mode of administration, and the particular site of the subject being treated, and the skin condition being treated. Those skilled in the art using conventional dosage-determination tests in view of the experimental data for a given composition may ascertain optimal dosages for a given set of conditions. This amount will vary depending upon a variety of factors, including but not limited to the characteristics of the compositions and formulations disclosed herein (including activity, pharmacokinetics, pharmacodynamics, and bioavailability thereof), the physiological condition of the subject treated (including age, sex, disease type and stage, general physical condition, responsiveness to a given dosage, and type of medication) or cells, the nature of the pharmaceutically acceptable carrier mg/kg or carriers in the formulation, and the route of administration. Further, an effective or therapeutically effective amount may vary depending on whether the one or more compositions and formulations disclosed herein is administered alone or in combination with other drug(s), other therapy/therapies or other therapeutic method(s) or modality/modalities. One skilled in the clinical and pharmacological arts will be able to determine an effective amount or therapeutically effective amount through routine experimentation, namely by monitoring a cell's or subject's response to administration of the one or more compositions and formulations disclosed herein and adjusting the dosage accordingly.

Dosage regimens using the first composition and the second composition may be adjusted to provide the optimum desired response. Dosage unit form, as used herein, refers to physically discrete units suited as unitary dosages for the subjects to be treated; each unit containing a predetermined quantity of the compositions disclosed herein, calculated to produce the desired therapeutic effect in association with the required pharmaceutical carrier. The specification for the dosage unit forms of the compositions disclosed herein are dictated by and directly dependent on (a) the characteristics of the composition and the particular therapeutic or prophylactic effect to be achieved, and (b) the limitations inherent in the art of compounding such a composition for the treatment a particular condition in a subject.

Thus, the skilled artisan would appreciate, based upon the disclosure provided herein, that the dose and dosing regimen using the compositions disclosed herein may be adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the presently disclosed methods.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. The embodiments disclosed herein are intended to encompass intra-subject dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

In some embodiments, the compositions may be used in combination with one or more additional compositions useful in treating skin conditions in a subject which are described below. When a combination therapy is used, the one or more additional compositions may be administered sequentially or simultaneously with the first composition and/or the second composition disclosed herein. In some embodiments, the additional compositions is administered to a subject prior to, at the same time as, or following administration of the first composition and/or the second composition disclosed herein. In some embodiments, the additional composition is administered to the subject prior to the administration of the first composition and/or the second composition disclosed herein. In some embodiments, the additional composition is administered to the subject at the same time the first composition and/or the second composition disclosed herein are administered to the subject. In some embodiments, the additional composition is administered to the subject following to the administration of the first composition and/or the second composition disclosed herein. Among the additional compositions that may be used according to any of the methods disclosed herein include, but are not limited to, cromolyn sodium (also known as sodium cromoglycate), topical alpha agonists (including, but not limited to, oxymetazoline hydrochloride, clonidine hydrochloride, apraclonidine hydrochloride, and brimonidine tartrate), topical antibiotics (including, but not limited to, tetracyclines [tetracycline, doxycycline, minocycline, sarecycline], clindamycin, and erythromycin), benzoyl peroxide, salicylic acid, azelaic acid, retinoids, topical anticholinergics (including, but not limited to, oxybutynin, glycopyrrolate, propantheline), topical prostaglandin analogs (including, but not limited to, latanoprost, bimatoprost, travoprost, and tafluprost), and topical hydroquinone or a combination of fluocinolone acetonide, hydroquinone, and tretinoin (sold as Tri-Luma® cream).

As will be understood by one skilled in the art, for all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Headings, e.g., (a), (b), (i) etc., are presented merely for ease of reading the specification and claims. The use of headings in the specification or claims does not require the steps or elements be performed in alphabetical or numerical order or the order in which they are presented.

The preparations and examples of a number of embodiments disclosed herein are intended to be illustrative and not limiting. All starting materials are available commercially or are described in the literature. All temperatures are reported in ° C.

### EXAMPLE 1

### Use of Spongilla and Ixekizumab for the Treatment of Plaque Psoriasis

Subjects 18 years of age and older having plaque psoriasis having a minimum body surface area involvement of 10%, a static Physician Global Assessment (sPGA) score of ≥3 in the overall assessment (plaque thickness/induration, erythema, and scaling) of psoriasis on a severity scale of 0 to 5, and a Psoriasis Area and Severity Index (PASI) score ≥12, are treated according to the regimen below.

Week 1: The subject's skin is washed and dried with a mild, hypoallergenic cleanser (eg, Cetaphil®). A prefilled syringe containing ixekizumab is removed from the refrigerator and allowed to reach room temperature (30 minutes) without removing the needle cap. 6 mL of 3% hydrogen peroxide USP is added to 2 grams of Spongilla powder, the mixture is stirred and is set aside. The Spongilla mixture is massaged into the areas of the subject's skin that are affected, taking care to avoid mucous membranes (eg, eyes, mouth, and nostrils), and the Spongilla composition is allowed to dry on the application area. The Spongilla composition is then removed using non-hypoallergenic wipes or water plus a cleanser. The composition comprising from 80 mg to 160 mg of ixekizumab is applied to the skin of the subject where Spongilla had been applied taking care to avoid mucous membranes (eg, eyes, mouth, and nostrils), and the ixekizumab is massaged into the subject's skin with, for example, a synthetic applicator or a health care provider's gloved finger. The ixekizumab composition is allowed to remain on the application area with the subject resting comfortably. After 15 minutes, the application area is washed with hypoallergenic wipes or water plus hypoallergenic cleanser. The procedure above is repeated once every two weeks in weeks 2, 4, 6, 8, 10, and 12 following the first application and then every 4 weeks thereafter.

Thereafter, subjects report an improvement in the plaque psoriasis affecting them, including a 75% or more reduction in the PASI composite score that takes into consideration both the percentage of body surface area affected and the nature and severity of psoriatic changes (induration, erythema and scaling) within the affected regions, and an sPGA of “0” (clear) or “1” (minimal).

### EXAMPLE 2

### Use of Spongilla and Ixekizumab for the Treatment of Psoriatic Arthritis

Subjects 18 years and older with active psoriatic arthritis (at least 3 swollen and at least 3 tender joints) despite treatment with nonsteroidal anti-inflammatory drug (NSAID), corticosteroid or disease modifying anti-rheumatic drug (DMARD) therapy are treated according to the regimen below.

Week 1: The subject's skin is washed and dried with a mild, hypoallergenic cleanser (eg, Cetaphil®). A prefilled syringe containing ixekizumab is removed from the refrigerator and allowed to reach room temperature (30 minutes) without removing the needle cap. 6 mL of 3% hydrogen peroxide USP is added to 3 grams of Spongilla powder, the mixture is stirred and is set aside. The Spongilla mixture is massaged into the areas of the subject's skin that are affected, taking care to avoid mucous membranes (eg, eyes, mouth, and nostrils), and the Spongilla composition is allowed to dry on the application area. The Spongilla composition is then removed using non-hypoallergenic wipes or water plus a cleanser. The composition comprising from 80 mg to 160 mg of ixekizumab is applied to the skin of the subject where Spongilla had been applied taking care to avoid mucous membranes (eg, eyes, mouth, and nostrils), and the ixekizumab is massaged into the subject's skin with a synthetic applicator. The ixekizumab composition is allowed to remain on the application area with the subject resting comfortably. After 15 minutes, the application area is washed with hypoallergenic wipes or water plus hypoallergenic cleanser. The procedure above is repeated once every two weeks in weeks 2, 4, 6, 8, 10, and 12 following the first application and then every 4 weeks thereafter. Thereafter, subjects report an improvement in their symptoms, including a lessening of joint tenderness and joint pain.

### EXAMPLE 3

### Use of Spongilla and Etanercept for the Treatment of Plaque Psoriasis

Subjects having chronic, stable plaque psoriasis involving at least 10% of the body surface area, a minimum Psoriasis Area and Severity Index (PASI) score of 10 are treated according to the regimen below. Subjects with guttate, erythrodermic, or pustular psoriasis and subjects with severe infections within 4 weeks of screening are excluded from treatment.

Week 1: The area of subject's skin to be treated is washed and dried with a mild, hypoallergenic cleanser (eg, Cetaphil®). Etanercept lyophilized powder is reconstituted aseptically with 1 mL of the supplied Sterile Bacteriostatic Water for Injection, USP (0.9% benzyl alcohol), giving a solution of 1 mL containing 25 mg of etanercept, and the reconstituted etanercept is set aside. 6 mL of 3% hydrogen peroxide USP is added to 3 grams of Spongilla powder, the mixture is stirred and is set aside. The Spongilla mixture is massaged into the areas of the subject's skin that are affected, taking care to avoid mucous membranes (eg, eyes, mouth, and nostrils), and the Spongilla composition is allowed to dry on the application area. The Spongilla composition is then removed using non-hypoallergenic wipes or water plus a cleanser. The etanercept composition comprising from about 25 mg to about 50 mg of etanercept is applied where Spongilla had been applied taking care to avoid mucous membranes (eg, eyes, mouth, and nostrils), and the etanercept is massaged into the subject's skin with a synthetic applicator. The etanercept composition is allowed to remain on the application area with the subject resting comfortably. After 15 minutes, the application area is washed with hypoallergenic wipes or water plus hypoallergenic cleanser. The procedure above is repeated once every week for maintenance purposes.

Thereafter, subjects report an improvement in the plaque psoriasis affecting them, including a 75% or more reduction in the PASI composite score that takes into consideration both the percentage of body surface area affected and the nature and severity of psoriatic changes (induration, erythema and scaling) within the affected regions, and a Physician Global Assessment (sPGA) of “0” (clear) or “1” (minimal).

### EXAMPLE 4

### Use of Spongilla and Dupilumab for the Treatment of Atopic Dermatitis

Subjects 18 years of age and older with moderate-to-severe atopic dermatitis (AD) not adequately controlled by current medications are treated according to the regimen below. Spongilla and dipliumab may be used for the prevention of atopic dermatitis in subjects, including, but not limited to, human subjects 18 years of age and older having a history of moderate-to-severe atopic dermatitis (AD) according to the regimen below.

An area of subject's skin to be treated is selected, washed and dried with a mild, hypoallergenic cleanser (eg, Cetaphil®). A prefilled syringe comprising dupilumab is removed from the refrigerator, allowed to reach room temperature (45 minutes) without removing the needle cap and is set aside. 6 mL of 3% hydrogen peroxide USP is added to 3 grams of Spongilla powder, the mixture is stirred and is set aside. The Spongilla mixture is massaged into the areas of the subject's skin to be treated, taking care to avoid mucous membranes (eg, eyes, mouth, and nostrils), and the Spongilla composition is allowed to dry on the application area. The Spongilla composition is then removed using non-hypoallergenic wipes or water plus a cleanser. The dupilumab composition comprising from about 300 mg to about 600 mg of dupilumab is applied where Spongilla had been applied taking care to avoid mucous membranes (eg, eyes, mouth, and nostrils), and the dupilumab is massaged into the subject's skin with a synthetic applicator. The dupilumab composition is allowed to remain on the application area with the subject resting comfortably. After 15 minutes, the application area is washed with hypoallergenic wipes or water plus hypoallergenic cleanser. The procedure above may be repeated once every week for maintenance purposes. Following this treatment regimen, the subject will experience a reduction in the number of lesions, and a reduction in the number of flare-ups, and recurrence.

1. A method of treating a disease or condition in a subject, comprising applying to the skin of the subject a first composition comprising Spongilla, and a second composition comprising a therapeutically effective amount of one or more drugs.

2. A method for delivering a drug into the skin of a subject, comprising applying to the skin of the subject a first composition comprising Spongilla, and a second composition comprising a therapeutically effective amount of one or more drugs.

3. A method for treating a skin disease or condition in a subject, comprising applying to the skin of the subject a first composition comprising Spongilla, and a second composition comprising a therapeutically effective amount of one or more drugs.

4. A method for treating or preventing one or more skin conditions or diseases in a subject comprising delivering a therapeutically or prophylactically effective amount of a drug to an intradermal compartment of the subject's skin by applying to the skin of the subject a first composition comprising Spongilla, and a second composition comprising a therapeutically effective amount of one or more drugs.

5. A method for administering a drug or other active agent to a subject, comprising applying to skin of the subject a first composition containing an effective amount of the drug or active agent, a second composition comprising Spongilla.

6. A method for enhancing skin permeation of a topically applied pharmacologically active compound which otherwise has a low rate of skin penetration, comprising applying to the skin of the subject a first composition comprising Spongilla, followed by application of a second composition to the skin of the subject, wherein said second composition comprises a therapeutically effective amount of said pharmacologically active compound.

7. A drug product comprising a first composition and a second composition, wherein said first composition comprises Spongilla, and said second composition comprises one or more drugs in an amount effective to treat a skin condition in a subject.

8. A kit comprising (a) a first composition comprising Spongilla, and (b) a second composition comprising one or more drugs in an amount effective to treat a skin condition in a subject.

9. The method according to claim 1, wherein said one or more drugs is not one or more of a botulinum toxin, an antibiotic, an anti-inflammatory, an antiseptic, or an anesthetic.

10. The method according to claim 1, wherein said disease or condition is a disease or condition of the skin of the subject.

11. The method according to claim 1, wherein said disease or condition is selected from plaque psoriasis, psoriatic arthritis, atopic dermatitis, acne vulgaris, acne rosacea type 1, acne rosacea type 2, psoriasis, hyperhidrosis, alopecia areata, androgenic alopecia, keloids, hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, melasma, rheumatoid arthritis, lichen planus, pityriasis rubra pilaris, ichthyosis, and palmoplantar pustulosis.

12. The method according to claim 1, wherein said one or more drugs is selected from a chemical compound, a mixture of chemical compounds, a biological macromolecule, or an extract made from biological materials.

13. The method according to claim 1, wherein said one or more drugs is a biological macromolecule.

14. The method according claim 13, wherein said one or more biological macromolecules is selected from a recombinant protein, a fusion protein, an antibody, a monoclonal antibody, a humanized monoclonal antibody, a bivalent antibody, an antibody fragment, an antibody-drug conjugate, a Fc fragment, a Fab fragment, a Fab′ fragment, a (Fab′)2 fragment, a Fv fragment, and a scFv fragment.

15. The method according to claim 14, wherein said one or more biological molecules is an antibody.

16. The method according to claim 15, wherein said antibody is a monoclonal antibody.

17. The method according to claim 16, wherein said monoclonal antibody is a humanized antibody.

18. The method according to claim 1, wherein said drug is selected from a T-cell co-stimulation modulator, an antagonist of one or more interleukin receptors, an antagonist of one or more interleukins, an interferon-gamma antagonist, a tissue necrosis factor-alpha antagonist, and a transforming growth factor-beta agonist.

19. The method according to claim 15, wherein said antibody is selected from one or more subclasses consisting of IgG1, IgG2, and IgG4.

20. The method according to claim 19, wherein said antibody is selected from one or more subclasses consisting of IgG1, IgG1/kappa, IgG1/lambda, IgG2, IgG2/kappa, and IgG4.

21. The method according to claim 15, wherein said antibody is selected from secukinumab, ixekizumab, adalimumab, brodalumab, guselkumab, ustekinumab, sarilumab, dupilumab, tildrakizumab, lebrikizumab, mepolizumab, fezakinumab, nemolizumab, risankizumab, BMS 981164, CMJ112, tralokinumab, cemiplimab, infliximab, basiliximab, daclizumab, efalizumab, ustekinumab, certolizumab pegol, ABX-IL8, omalizumab, mirikizumab, and MSB0010841 (ALX-0761).

22. The method according to claim 14, wherein said biologic macromolecules is a protein.

23. The method according to claim 22, wherein said protein is selected from etanercept, abatacept, rilonacept, and anakinra.

24. The method according to claim 22, wherein said protein is a fusion protein.

25. The method according to claim 24, wherein said fusion protein is selected from etanercept, abatacept, and rilonacept.

26. The method according to claim 18, wherein said one or more interleukin receptors is selected from IL-1R, IL-1RA, IL-1RB, IL-2R, IL-4R, IL-5R, IL-6R, IL-10R, IL-12R, IL-13R, IL-17R, IL-17RA, IL-21R, IL-22R, IL-23R, IL-31R, and IL-35R.

27. The method according to claim 1, wherein the first composition comprises Spongilla in the form of a powder.

28. The method according to claim 27, wherein the Spongilla is in the form of a powder comprising particles that are substantially uniform in size.

29. The method of claim 28, wherein not less than 50% of the particles comprising the Spongilla powder pass through a US 70-mesh screen.

30. The method of claim 29, wherein the particles comprising the Spongilla powder have an average length of from about 50 μm to about 500 μm.

31. The method of claim 29, wherein the particles comprising the Spongilla powder have an average diameter of from about 5 μm to about 50 μm.

32. The method of claim 29, wherein the particles comprising the Spongilla powder have an aspect ratio of from about 1 to 100.

33. The method of claim 1, wherein the first composition has a residual moisture content of not more than about 10%.

34. The method of claim 1, wherein the first composition has a combined aerobic and anaerobic microbial content of not more than about 25×10⁴ colony-forming units per gram (CFU/g).

35. The method of claim 1, wherein the first composition has a combined yeast and mold content of not more than about 25×10⁴ colony-forming units per gram (CFU/g).

36. The method of claim 1, wherein the amount of Coliform bacteria in the first composition is not more than about 25×10⁴ colony-forming units per gram (CFU/g).

37. The method of claim 1, wherein the amount of Salmonella in the first composition is not more than about 25×10⁴ colony-forming units per gram (CFU/g).

38. The method of claim 1, wherein the amount of Pseudomonas aeruginosa bacteria in the first composition is not more than about 25×10⁴ colony-forming units per gram (CFU/g).

39. The method of claim 1, wherein the amount of Staphylococcus aureus bacteria in the first composition is not more than about 25×10⁴ colony-forming units per gram (CFU/g).

40. The method of claim 1, wherein the first composition is packaged prior to use.

41. The method according to claim 1, wherein the first composition is prepared by heating to at least about 70° C. prior to being packaged.

42. The method according to claim 41, wherein the first composition is heated to at least about 70° C. for at least about 5 minutes prior being packaged.

43. The method according to claim 1, wherein the first composition is prepared by treating with gamma radiation or heat prior to being packaged.

44. The method according to claim 1, wherein the Spongilla is Spongilla lacustris.

45. The method according to claim 1, wherein the second composition is applied to the skin of the subject in the form of a pharmaceutical composition comprising the second composition and one or more pharmaceutically acceptable carriers or excipients.

46. The method according to claim 45, wherein the second composition is in the form of a solution, an aqueous solution, a powder, or a gel.

47. The method according to claim 1, wherein the amount of the first composition comprising Spongilla applied to the skin of the subject is from about 0.5 grams to about 50 grams.

48. The method according to claim 1, wherein the first composition is applied to the skin of the subject in the form of a paste.

49. The method according to claim 48, wherein the paste further comprises water or saline.

50. The method according to claim 48, wherein the paste is prepared by mixing a powder comprising Spongilla and water or saline.

51. The method according to claim 1, wherein the first composition is applied to the skin of the subject prior to the second composition being applied to the skin of the subject.

52. The method according to claim 51, wherein the first composition is applied to the skin of the subject and is permitted to dry on the skin of the subject prior to application of the second composition to the skin of the subject.

53. The method according to claim 51, wherein the first composition is washed off the skin of the subject prior to the second composition being applied to the skin of the subject.

54. The method according to claim 52, wherein the first composition is applied to the skin of the subject in the form of an aqueous paste.

55. The method according to claim 51, wherein the second composition is permitted to dry on the skin of the subject following application to the skin of the subject.

56. The method according to claim 1, wherein the second composition is applied to the skin of the subject prior to the first composition being applied to the skin of the subject.

57. The method according to claim 55, wherein the second composition is permitted to dry on the skin of the subject prior to the first composition being applied to the skin of the subject.

58. The method of claim 56, wherein the first composition is permitted to dry on the skin of the subject.

59. The method according to claim 1, wherein the first composition and the second composition are mixed together and the resulting mixture is applied to the skin of the subject.

60. The method according to claim 1, wherein the first composition and the second composition are applied to the skin of the subject at least once per week.

61. The method according to claim 1, wherein the first composition is applied to the skin of the subject at least once per week for at least one week.

62. The method according to claim 2, wherein said drug is delivered to the dermis of the subject.

63. The method of claim 9, wherein said anti-inflammatory is selected from non-steroidal anti-inflammatories and steroidal anti-inflammatories.

64. A kit comprising a first composition comprising Spongilla, and a second composition comprising a therapeutically effective amount of one or more drugs.

65. The kit according to claim 64 for use in treating a disease or condition in a subject.

66. The kit according to claim 64, wherein said one or more drugs is not a botulinum toxin, an antibiotic, an anti-inflammatory, an antiseptic, an anesthetic.

67. The kit according to claim 65, wherein said disease or condition is a disease or condition of the skin of the subject.

68. The kit according to claim 67, wherein said disease or condition is selected from plaque psoriasis, psoriatic arthritis, atopic dermatitis, acne vulgaris, acne rosacea type 1, acne rosacea type 2, psoriasis, hyperhidrosis, alopecia areata, androgenic alopecia, keloids, hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, melasma, rheumatoid arthritis, lichen planus, pityriasis rubra pilaris, ichthyosis, and palmoplantar pustulosis.

69. The kit according to claim 64, wherein said one or more drugs is selected from a chemical compound, a mixture of chemical compounds, a biological macromolecule, or an extract made from biological materials.

70. The kit according to claim 69, wherein said one or more drugs is a biological macromolecule.

71. The kit according to claim 70, wherein said one or more biological macromolecules is selected from a recombinant protein, a fusion protein, an antibody, a monoclonal antibody, a humanized monoclonal antibody, a bivalent antibody, an antibody fragment, an antibody-drug conjugate, a Fc fragment, a Fab fragment, a Fab′ fragment, a (Fab′)2 fragment, a Fv fragment, and a scFv fragment.

72. The kit according to claim 71, wherein said one or more biological molecules is an antibody.

73. The kit according to claim 71, wherein said antibody is a monoclonal antibody.

74. The kit according to claim 71, wherein said monoclonal antibody is a humanized antibody.

75. The kit according to claim 70, wherein said drug is selected from a T-cell co-stimulation modulator, an antagonist of one or more interleukin receptors, an antagonist of one or more interleukins, an interferon-gamma antagonist, a tissue necrosis factor-alpha antagonist, and a transforming growth factor-beta agonist.

76. The kit according to claim 72, wherein said antibody is selected from one or more subclasses consisting of IgG1, IgG2, and IgG4.

77. The kit according to claim 76, wherein said antibody is selected from one or more subclasses consisting of IgG1, IgG1/kappa, IgG1/lambda, IgG2, IgG2/kappa, and IgG4.

78. The kit according to claim 73, wherein said antibody is selected from secukinumab, ixekizumab, adalimumab, brodalumab, guselkumab, ustekinumab, sarilumab, dupilumab, tildrakizumab, lebrikizumab, mepolizumab, fezakinumab, nemolizumab, risankizumab, BMS 981164, CMJ112, tralokinumab, cemiplimab, infliximab, basiliximab, daclizumab, efalizumab, ustekinumab, certolizumab pegol, ABX-IL8, omalizumab, mirikizumab, and MSB0010841 (ALX-0761).

79. The kit according to claim 71, wherein said biologic macromolecules is a protein.

80. The kit according to claim 79, wherein said protein is selected from etanercept, abatacept, rilonacept, and anakinra.

81. The kit according to claim 79, wherein said protein is a fusion protein.

82. The kit according to claim 81, wherein said fusion protein is selected from etanercept, abatacept, and rilonacept.

83. The kit according to claim 75, wherein said one or more interleukin receptors is selected from IL-1R, IL-1RA, IL-1RB, IL-2R, IL-4R, IL-5R, IL-6R, IL-10R, IL-12R, IL-13R, IL-17R, IL-17RA, IL-21R, IL-22R, IL-23R, IL-31R, and IL-35R.

84. The method or kit according to any one of the prior claims, wherein the first composition comprises Spongilla in the form of a powder.

85. The method or kit according to any one of the prior claims, wherein the Spongilla is in the form of a powder comprising particles that are substantially uniform in size.

86. The method or kit according to any one of the prior claims, wherein not less than 50% of the particles comprising the Spongilla powder pass through a US 70-mesh screen.

87. The method or kit according to any one of the prior claims, wherein the particles comprising the Spongilla powder have an average length of from about 50 μm to about 500 μm.

88. The method or kit according to any one of the prior claims, wherein the particles comprising the Spongilla powder have an average diameter of from about 5 μm to about 50 μm.

89. The method or kit according to any one of the prior claims, wherein the particles comprising the Spongilla powder have an aspect ratio of from about 1 to 100.

90. The method or kit according to any one of the prior claims, wherein the first composition has a residual moisture content of not more than about 10%.

91. The method or kit according to any one of the prior claims, wherein the first composition has a combined aerobic and anaerobic microbial content of not more than about 25×10⁴ colony-forming units per gram (CFU/g).

92. The method or kit according to any one of the prior claims, wherein the first composition has a combined yeast and mold content of not more than about 25×10⁴ colony-forming units per gram (CFU/g).

93. The method or kit according to any one of the prior claims, wherein the amount of Coliform bacteria in the first composition is not more than about 25×10⁴ colony-forming units per gram (CFU/g).

94. The method or kit according to any one of the prior claims, wherein the amount of Salmonella in the first composition is not more than about 25×10⁴ colony-forming units per gram (CFU/g).

95. The method or kit according to any one of the prior claims, wherein the amount of Pseudomonas aeruginosa bacteria in the first composition is not more than about 25×10⁴ colony-forming units per gram (CFU/g).

96. The method or kit according to any one of the prior claims, wherein the amount of Staphylococcus aureus bacteria in the first composition is not more than about 25×10⁴ colony-forming units per gram (CFU/g).

97. The method or kit according to any one of the prior claims, wherein the first composition is packaged prior to use.

98. The method or kit according to any one of the prior claims, wherein the first composition is prepared by heating to at least about 70° C. prior to being packaged.

99. The method or kit according to any one of the prior claims, wherein the first composition is heated to at least about 70° C. for at least about 5 minutes prior being packaged.

100. The method or kit according to any one of the prior claims, wherein the first composition is prepared by treating with gamma radiation prior to being packaged.

101. The method or kit according to any one of the prior claims, wherein the first composition further comprises an aqueous solution of hydrogen peroxide.

102. The method or kit according to any one of the prior claims, wherein the hydrogen peroxide is at a concentration of about 3%.

103. The method or kit according to any one of the prior claims, wherein the method further comprises applying a third composition to the skin of the subject.

104. The method or kit according to any one of the prior claims, wherein the third composition comprises hydrogen peroxide.

105. The method or kit according to any one of the prior claims, wherein the hydrogen peroxide is at a concentration of about 3%.

106. The method or kit according to any one of the prior claims, wherein the Spongilla is Spongilla lacustris.

107. The method or kit according to any one of the prior claims, wherein the second composition comprising one or more drugs is in the form of an aqueous solution.

108. The method or kit according to any one of the prior claims, wherein the second composition is applied to the skin of the subject in the form of a solution.

109. The method or kit according to any one of the prior claims, wherein the second composition is in the form of an aqueous solution.

110. The method or kit according to any one of the prior claims, wherein the amount of the first composition comprising Spongilla applied to the skin of the subject is from about 0.5 grams to about 50 grams.

111. The method or kit according to any one of the prior claims, wherein the first composition is applied to the skin of the subject in the form of a paste.

112. The method or kit according to any one of the prior claims, wherein the paste further comprises water or saline.

113. The method according to any one of the prior claims, wherein the first composition is applied to the skin of the subject prior to the second composition being applied to the skin of the subject.

114. The method according to any one of the prior claims, wherein the first composition is applied to the skin of the subject and is permitted to dry on the skin of the subject prior to application of the second composition to the skin of the subject.

115. The method or kit according to any one of the prior claims, wherein the first composition is applied to the skin of the subject in the form of an aqueous paste.

116. The method or kit according to any one of the prior claims, wherein the aqueous paste further comprises hydrogen peroxide.

117. The method according to any one of the prior claims, wherein the second composition is permitted to dry on the skin of the subject following application to the skin of the subject.

118. The method according to any one of the prior claims, wherein the second composition is applied to the skin of the subject prior to the first composition being applied to the skin of the subject.

119. The method according to any one of the prior claims, wherein the second composition is permitted to dry on the skin of the subject prior to the first composition being applied to the skin of the subject.

120. The method or kit according to any one of the prior claims, wherein the first composition is applied to the skin of the subject in the form of an aqueous paste.

121. The method according to any one of the prior claims, wherein the first composition comprising Spongilla is applied to the skin of the subject at least once per week for at least one week.

122. The method according to any one of the prior claims, wherein the first composition comprising Spongilla is applied to the skin of the subject once per week for 6 weeks.

123. The method according to any one of the prior claims, wherein the skin of the subject is cleaned using a non-comedogenic cleanser, water, or a combination of a non-comedogenic cleanser and water following application of the first composition comprising Spongilla.

124. The method according to any one of the prior claims, wherein the skin of the subject is cleaned using a non-comedogenic cleanser, water, or a combination of a non-comedogenic cleanser and water following application of the second composition to the skin of the subject.

125. The method according to any one of the prior claims, wherein the skin condition in the subject is selected from plaque psoriasis, psoriatic arthritis, atopic dermatitis, acne vulgaris, acne rosacea type 1, acne rosacea type 2, psoriasis, hyperhidrosis, alopecia areata, androgenic alopecia, keloids, and hypertrophic scars, hidradenitis suppurativa, Raynaud phenomenon, post-herpetic neuralgia, Hailey-Hailey disease, IgA bullous dermatosis, epidermolysis bullosa Simplex Weber-Cockane, Darier disease, pachyonchia congenita, aquagenic keratoderma, notalgia paresthetic, pompholyx (dyshidrotic eczema), chromhidrosis and bromhidrosis, eccrine nevus, facial rhytides atrohpic acne scars, and melasma.

126. A method of treating a condition in a subject substantially as hereinbefore described with reference to any one of the Examples or described herein.

127. The method according to claim 126, wherein the subject is a human.

128. The method according to claim 127, wherein the condition in the subject is a skin condition.

129. A kit substantially as hereinbefore described with reference to any one of the Examples or as described herein.
